(19) United States
(12) Patent Application Publication
(10) Pub. No.: US 2005/0256107 A1
(43) Pub. Date: Nov. 17, 2005

(54) New substituted thiophene carboxamides, process for their preparation and their use as medicaments

(75) Inventors: Roland Pfau, Biberach (DE); Henning Priepke, Warthausen (DE); Kai Gerlach, Biberach (DE); Wolfgang Wienen, Biberach (DE); Annette Schuler-Metz, Ulm (DE); Herbert Nar, Ochsenhausen (DE); Sandra Handschuh, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/125,493

(22) Filed: May 10, 2005

(30) Foreign Application Priority Data
May 13, 2004 (EP) 04011387

(51) Int. Cl. A61K 31/551; A61K 31/501; A61K 31/496; A61K 31/453; A61K 31/416; A61K 31/4025; C07D 49/02

(52) U.S. Cl. 514/218; 540/575; 514/252.01; 514/242; 514/252.13; 514/326; 514/404; 514/422; 544/238; 544/183; 544/379; 546/207; 548/364.1; 548/527

(57) ABSTRACT

The present invention relates to new substituted thiophene-2-carboxylic acid amides of general formula 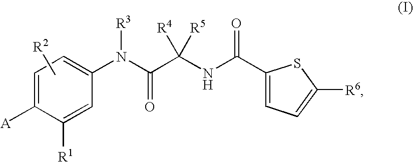
wherein A, and R¹ to R^{8c} are defined as in claim 1, the tautomers, the enantiomers, the diastereomers, the mixtures thereof and the salts thereof, particularly the physiologically acceptable salts thereof with inorganic or organic acids or bases, which have valuable properties.

The present invention relates to new substituted thiophene-2-carboxylic acid amides of general formula 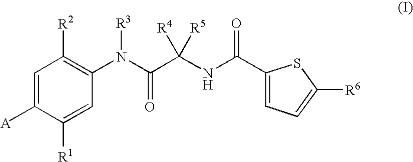
the tautomers, the enantiomers, the diastereomers, the mixtures thereof and the salts thereof, particularly the physiologically acceptable salts thereof with inorganic or organic acids or bases, which have valuable properties.

The compounds of the above general formula I as well as the tautomers, the enantiomers, the diastereomers, the mixtures thereof and the salts thereof, particularly the physiologically acceptable salts thereof with inorganic or organic acids or bases, and their stereoisomers have valuable pharmacological properties, particularly an antithrombotic activity and a factor Xa-inhibiting activity.

The present application thus relates to the new compounds of the above general formula I, the preparation thereof, the pharmaceutical compositions containing the pharmacologically effective compounds, the preparation and use thereof.

A 1st embodiment of the present invention comprises those compounds of general formula I, wherein
A denotes a group of general formula 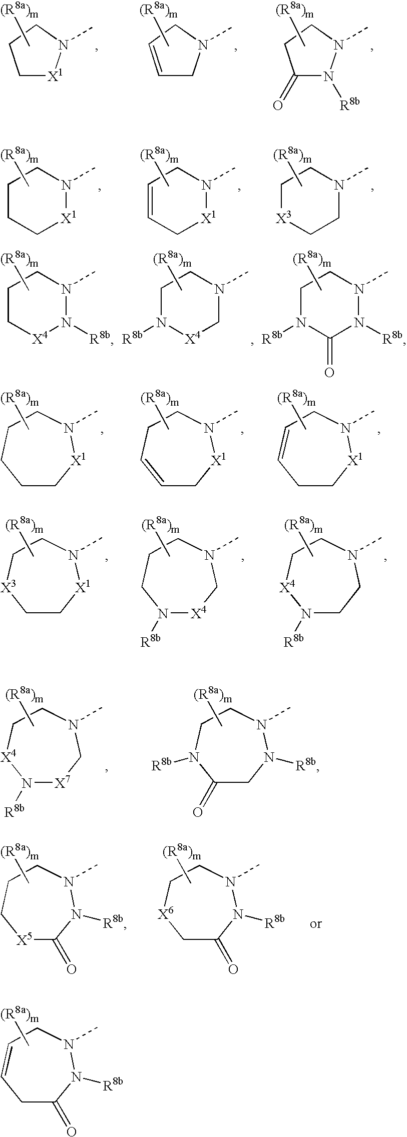
   wherein
m is the number 1 or 2,
R^{8a} in each case independently of one another denotes a hydrogen or halogen atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, Cl, Br, I, O or N optionally introduced with R^{8a} as substituent are not separated by precisely one carbon atom from a heteroatom selected from among N, O, S,
R^{8b} in each case independently of one another denotes a hydrogen atom or a C₁₋₅-alkyl group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X¹ denotes an oxygen atom or a —CH₂, —CHR^{8a}— or —NR^{8c}— group,
X² denotes an oxygen atom or a —NR^{8b}— group,
X³ denotes an oxygen or sulphur atom, a —NR^{8c}— group,
X⁴ denotes a carbonyl or sulphonyl group,
X⁵ denotes an oxygen atom, a —NR^{8b}— or methylene group,
X⁶ denotes an oxygen or sulphur atom or a —NR^{8c}— group,
X⁷ denotes a methylene or carbonyl group,
R¹ denotes a hydrogen or halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R³ denotes a hydrogen atom or a C₁₋₃-alkyl group,
R⁴ and R⁵ in each case independently of one another denote
   a hydrogen atom, a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a nitrile, hydroxy, a C₁₋₅-alkyloxy group, while the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₁₋₅-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino group,
   a carboxy, aminocarbonyl, C₁₋₅-alkylaminocarbonyl, C₃₋₆-cycloalkylaminocarbonyl, di-(C₁₋₅-alkyl)-aminocarbonyl, C₁₋₅-alkoxycarbonyl, C₄₋₆-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from the group consisting of halogen atoms, C₁₋₅-alkyl, di-(C₁₋₅-alkyl)-amino, hydroxy, C₁₋₅-alkyloxy, mono-, di- or trifluoromethoxy, carboxy and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      wherein in 4- to 7-membered cyclic groups in the cyclic moiety a methylene group may optionally be replaced by an —N(R^{8c})— group, an oxygen or sulphur atom or a —S(O)— or —S(O)₂— group, or
      wherein in 4- to 7-membered cyclic groups in the cyclic moiety two adjacent methylene groups may together optionally be replaced by a —C(O)N(R^{8b})— or —S(O)₂N(R^{8b})— group, or
      wherein in 6- to 7-membered cyclic groups in the cyclic moiety three adjacent methylene groups may optionally be replaced together by a substituted —OC(O)N(R^{8b})— or —N(R^{8b})C(O)N(R^{8b})— or
      —N(R^{8b})S(O)₂N(R^{8b})— group,
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms selected from among oxygen and nitrogen are separated from one another by precisely one optionally substituted —CH₂— group, is excluded,
      while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group,
   while one of the methylene groups of a C₄₋₈-cycloalkyl group may be replaced by an oxygen or sulphur atom or an —N(R^{8c})—, or a carbonyl, sulphinyl or sulphonyl group, and/or
   two directly adjacent methylene groups of a C₄₋₈-cycloalkyl group may be replaced together by a —C(O)N(R^{8b})— or —S(O)₂N(R^{8b})— group, and/or
   three directly adjacent methylene groups of a C₆₋₈-cycloalkyl group may be replaced together by a —OC(O)N(R^{8b})—, —N(R^{8b})C(O)N(R^{8b})— or —N(R^{8b})S(O)₂N(R^{8b})— group,
   while one or two carbon atoms of a C₃₋₈-cycloalkyl group may optionally be substituted independently of one another by in each case one or two identical or different halogen atoms or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
   while one or two carbon atoms of a C₃₋₈-cycloalkenyl group may optionally be substituted independently of one another by in each case one or two identical or different C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl group,
   and one or two carbon atoms of a C₄₋₈-cycloalkenyl group which are not bound to another carbon atom by a double bond may optionally be substituted independently of one another by two identical or different halogen atoms or a hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonyl-amino group,
   with the proviso that a C₃₋₈-cycloalkyl or C₃₋₈-cycloalkenyl group of this kind, formed from R⁴ and R⁵ together,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among an oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a hydrogen, fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term ‘heteroaryl group’ mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while the alkyl, alkenyl, alkynyl and alkoxy groups contained in the foregoing definitions which have more than two carbon atoms, unless otherwise stated, may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

Examples of monocyclic heteroaryl groups are the pyridyl, N-oxy-pyridyl, pyrazolyl, pyridazinyl, pyrimidinyl, pyrazinyl, [1,2,3]triazinyl, [1,3,5]triazinyl, [1,2,4]triazinyl, pyrrolyl, imidazolyl, [1,2,4]triazolyl, [1,2,3]triazolyl, tetrazolyl, furanyl, isoxazolyl, oxazolyl, [1,2,3]oxadiazolyl, [1,2,4]oxadiazolyl, furazanyl, thiophenyl, thiazolyl, isothiazolyl, [1,2,3]thiadiazolyl, [1,2,4]thiadiazolyl or [1,2,5]thiadiazolyl group.

Examples of bicyclic heteroaryl groups are the benzimidazolyl, benzofuranyl, benzo[c]furanyl, benzothiophenyl, benzo[c]thiophenyl, benzothiazolyl, benzo[c]-isothiazolyl, benzo[d]isothiazolyl, benzoxazolyl, benzo[c]isoxazolyl, benzo[d]-isoxazolyl, benzo[1,2,5]oxadiazolyl, benzo[1,2,5]thiadiazolyl, benzo[1,2,3]thiadiazolyl, benzo[d][1,2,3]triazinyl, benzo[1,2,4]triazinyl, benzotriazolyl, cinnolinyl, quinolinyl, N-oxy-quinolinyl, isoquinolinyl, quinazolinyl, N-oxy-quinazolinyl, quinoxalinyl, phthalazinyl, indolyl, isoindolyl or 1-oxa-2,3-diaza-indenyl group.

Examples of the C₁₋₆-alkyl groups mentioned hereinbefore in the definitions are the methyl, ethyl, 1-propyl, 2-propyl, n-butyl, sec-butyl, tert-butyl, 1-pentyl, 2-pentyl, 3-pentyl, neo-pentyl, 3-methyl-2-butyl, 1-hexyl, 2-hexyl, 3-hexyl, 3-methyl-2-pentyl, 4-methyl-2-pentyl, 3-methyl-3-pentyl, 2-methyl-3-pentyl, 2,2-dimethyl-3-butyl or 2,3-dimethyl-2-butyl group.

Examples of the C₁₋₅-alkyloxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy, 3-pentyloxy or neo-pentyloxy group.

Examples of the C₂₋₆-alkenyl groups mentioned hereinbefore in the definitions are the ethenyl, 1-propen-1-yl, 2-propen-1-yl, 1-buten-1-yl, 2-buten-1-yl, 3-buten-1-yl, 1-penten-1-yl, 2-penten-1-yl, 3-penten-1-yl, 4-penten-1-yl, 1-hexen-1-yl, 2-hexen-1-yl, 3-hexen-1-yl, 4-hexen-1-yl, 5-hexen-1-yl, but-1-en-2-yl, but-2-en-2-yl, but-1-en-3-yl, 2-methyl-prop-2-en-1-yl, pent-1-en-2-yl, pent-2-en-2-yl, pent-3-en-2-yl, pent-4-en-2-yl, pent-1-en-3-yl, pent-2-en-3-yl, 2-methyl-but-1-en-1-yl, 2-methyl-but-2-en-1-yl, 2-methyl-but-3-en-1-yl, 2-ethyl-prop-2-en-1-yl, hex-1-en-2-yl, hex-2-en-2-yl, hex-3-en-2-yl, hex-4-en-2-yl, hex-5-en-2-yl, hex-1-en-3-yl, hex-2-en-3-yl, hex-3-en-3-yl, hex-4-en-3-yl, hex-5-en-3-yl, hex-1-en-4-yl, hex-2-en-4-yl, hex-3-en-4-yl, hex-4-en-4-yl, hex-5-en-4-yl, 4-methyl-pent-1-en-3-yl, 3-methyl-pent-1-en-3-yl, 2-methyl-pent-1-en-3-yl, 2,3-dimethyl-but-1-en-3-yl, 3,3-dimethyl-but-1-en-2-yl or 2-ethyl-but-1-en-3-yl group,

Examples for the mentioned hereinbefore in the definitions C₂₋₆-alkynyl groups are the ethynyl, 1-propynyl, 2-propynyl, 1-butyn-1-yl, 1-butyn-3-yl, 2-butyn-1-yl, 3-butyn-1-yl, 1-pentyn-1-yl, 1-pentyn-3-yl, 1-pentyn-4-yl, 2-pentyn-1-yl, 2-pentyn-3-yl, 3-pentyn-1-yl, 4-pentyn-1-yl, 2-methyl-1-butyn-4-yl, 3-methyl-1-butyn-1-yl, 3-methyl-1-butyn-3-yl, 1-hexyn-1-yl, 2-hexyn-1-yl, 3-hexyn-1-yl, 4-hexyn-1-yl, 5-hexyn-1-yl, 1-hexyn-3-yl, 1-hexyn-4-yl, 1-hexyn-5-yl, 2-hexyn-4-yl, 2-hexyn-5-yl, 3-hexyn-5-yl, 3-methyl-1-pentyn-3-yl, 4-methyl-1-pentyn-3-yl, 3-methyl-1-pentyn-4-yl, 4-methyl-1-pentyn-4-yl, 4-methyl-2-pentyn-4-yl, 4-methyl-2-pentyn-1-yl, 2,2-dimethyl-3-butyn-1-yl or 2-ethyl-3-butyn-1-yl group.

By a group which may be converted in vivo into a carboxy group is meant for example a carboxy group esterified with an alcohol wherein the alcoholic moiety preferably denotes a C₁₋₆-alkanol, a phenyl-C₁₋₃-alkanol, a C₃₋₉-cycloalkanol, a C₅₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol or an alcohol of formula
R⁹—CO—O—(R¹⁰CR¹¹)—OH,
wherein
R⁹ denotes a C₁₋₈-alkyl, C₅₋₇-cycloalkyl, phenyl or phenyl-C₁₋₃-alkyl group,
R¹⁰ denotes a hydrogen atom, a C₁₋₃-alkyl, C₅₋₇-cycloalkyl or phenyl group and
R¹¹ denotes a hydrogen atom or a C₁₋₃-alkyl group.

Preferred groups which may be cleaved from a carboxy group in vivo include a C₁₋₆-alkoxy group such as the methoxy, ethoxy, n-propyloxy, isopropyloxy, n-butyloxy, n-pentyloxy, n-hexyloxy or cyclohexyloxy group or a phenyl-C₁₋₃-alkoxy group such as the benzyloxy group.

By a group which may be converted in vivo into a hydroxyl group is meant for example a hydroxyl group esterified with a carboxylic acid wherein the carboxylic acid moiety is preferably a C₁₋₇-alkanoic acid, a phenyl-C₁₋₃-alkanoic acid, a C₃₋₉-cycloalkylcarboxylic acid, a C₅₋₇-cycloalkenecarboxylic acid, a C₃₋₇-alkenoic acid, a phenyl-C₃₋₅-alkenoic acid, a C₃₋₇-alkynoic acid or phenyl-C₃₋₅-alkynoic acid, while individual methylene groups of the carboxylic acid group may be replaced by oxygen atoms, with the proviso that no bond to the oxygen atom starts from a carbon atom which carries a double or triple bond.

Examples of preferred groups which may be cleaved in vivo from a hydroxyl group include a C₁₋₇-acyl group such as the formyl, acetyl, n-propionyl, isopropionyl, n-propanoyl, n-butanoyl, n-pentanoyl, n-hexanoyl or cyclohexylcarbonyl group or a benzoyl group and also a methoxyacetyl, 1-methoxypropionyl, 2-methoxypropionyl or 2-methoxy-ethoxyacetyl group.

The compounds of general formula I, wherein A, R⁴ and/or R⁵ contains a group which may be converted in vivo into a carboxy or hydroxyl group are prodrugs for those compounds of general formula I wherein A, R⁴ and/or R⁵ contains a carboxy or hydroxyl group.

A 2nd embodiment of the present invention comprises those compounds of general formula I, wherein
A denotes a group of general formula 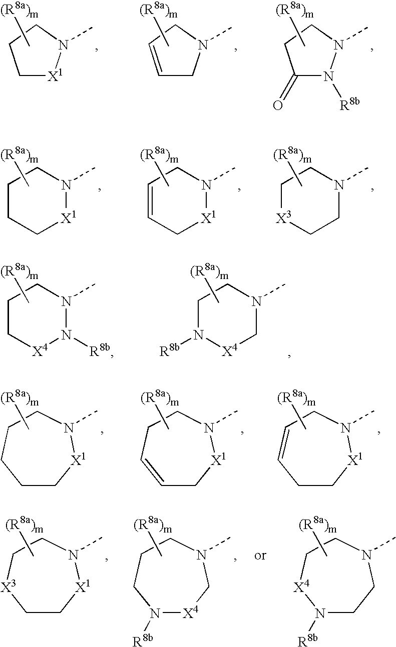
   wherein
m is the number 1 or 2,
R^{8a} each independently of one another denote a hydrogen or halogen atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, Cl, Br, I, O or N optionally introduced with R^{8a} as substituent are not separated by precisely one carbon atom from a heteroatom selected from among N, O, S,
R^{8b} in each case independently of one another denotes a hydrogen atom or a C₁₋₅-alkyl group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X¹ denotes an oxygen atom or a —CH₂—, —CHR^{8a}— or —NR^{8c}— group,
X³ denotes an oxygen or sulphur atom, a —NR^{8c}— group,
X⁴ denotes a carbonyl or sulphonyl group,
R¹ denotes a halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R³ denotes a hydrogen atom or a C₁₋₃-alkyl group,
R⁴ and R⁵ in each case independently of one another denote
   a hydrogen atom, a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a nitrile, hydroxy, a C₁₋₅-alkyloxy group, while the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino group,
   a carboxy, aminocarbonyl, C₁₋₅-alkylaminocarbonyl, C₃₋₆-cycloalkylaminocarbonyl, di-(C₁₋₁₅-alkyl)-aminocarbonyl, C₁₋₅-alkoxycarbonyl, C₄₋₆-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      while in 4- to 7-membered cyclic groups in the cyclic moiety a methylene group may optionally be replaced by an —N(R^{8c})— group, an oxygen or sulphur atom or a —S(O) or —S(O)₂ group, or in 4- to 7-membered cyclic groups in the cyclic moiety two adjacent methylene groups together may optionally be replaced by a —C(O)N(R^{8b})— or —S(O)₂N(R^{8b})— group, or
      wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or
      —N(R^{8b})S(O)₂N(R^{8b})— group,
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group is excluded,
      while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group,
   while one of the methylene groups of a C₄₋₈-cycloalkyl group may be replaced by an oxygen or sulphur atom or an —N(R^{8c})—, or a carbonyl, sulphinyl or sulphonyl group, and/or
   two directly adjacent methylene groups of a C₄₋₈-cycloalkyl group may together be replaced by a —C(O)N(R^{8b})— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or —N(R^{8b})S(O)₂N(R^{8b})— group,
   while one or two carbon atoms of a C₃₋₈-cycloalkyl group may optionally be substituted independently of one another in each case by one or two identical or different halogen atoms or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
   while one or two carbon atoms of a C₃₋₈-cycloalkenyl group may optionally be substituted independently of one another in each case by one or two identical or different C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl group,
   and one or two carbon atoms of a C₄₋₈-cycloalkenyl group which are not bound to another carbon atom by a double bond may each optionally be substituted independently of one another by one or two identical or different halogen atoms or hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonyl-amino groups,
   with the proviso that a C₃₋₈-cycloalkyl or C₃₋₈-cycloalkenyl group of this kind, formed together from R⁴ and R⁵,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while unless otherwise stated the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 3rd embodiment of the present invention comprises those compounds of general formula I, wherein
A denotes a group of general formula 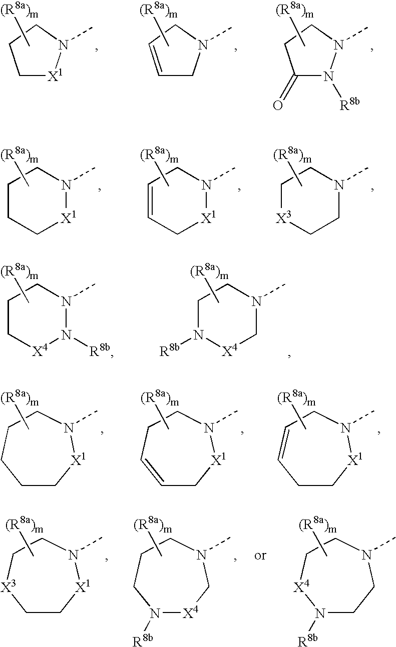
   wherein
m is the number 1 or 2,
R^{8a} each independently of one another denote a hydrogen or halogen atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, Cl, Br, I, O or N optionally introduced with R^{8a} as substituent are not separated by precisely one carbon atom from a heteroatom selected from among N, O, S,
R^{8b} in each case independently of one another denotes a hydrogen atom or a C₁₋₅-alkyl group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X¹ denotes an oxygen atom or a —CH₂—, —CHR^{8a}— or —NR^{8c}— group,
X³ denotes an oxygen atom, or a —NR^{8c}— group,
X⁴ denotes a carbonyl or sulphonyl group,
R¹ denotes a halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, or a nitrile group,
R² denotes a hydrogen or halogen atom or a methyl group,
R³ denotes a hydrogen atom or a methyl group,
R⁴ denotes a hydrogen atom, a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a nitrile, hydroxy, a C₁₋₅-alkyloxy group, while the hydrogen atoms of the C₁₋₅-alkyloxy group may optionally be wholly or partly replaced by fluorine atoms, a benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino or N-(C₁₋₅-alkylsulphonyl)-C₁₋₅-alkylamino group,
   a carboxy, aminocarbonyl, C₁₋₅-alkylaminocarbonyl, C₃₋₆-cycloalkylamino-carbonyl, di-(C₁₋₅-alkyl)-aminocarbonyl, C₁₋₅-alkoxycarbonyl, C₄₋₆-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from among halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      while in 4- to 7-membered cyclic groups in the cyclic moiety a methylene group may optionally be replaced by an —N(R^{8c})— group, an oxygen or sulphur atom or a —S(O)— or —S(O)₂— group, or
      while in 4- to 7-membered cyclic groups in the cyclic moiety two adjacent methylene groups together may optionally be replaced by a —C(O)N(R^{8b})— or —S(O)₂N(R^{8b})— group, or
   wherein in 6- to 7-membered cyclic groups in the cyclic moiety three adjacent methylene groups together may optionally be replaced by a substituted —OC(O)N(R^{8b}) or N(R^{8b})C(O)N(R^{8b}) or N(R^{8b})S(O)₂N(R^{8b})— group,
   with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group is excluded,
   while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case,
R⁵ denotes a hydrogen atom, a C₂₋₆-alkenyl or C₂₋₆-alkynyl group,
   a straight-chain or branched C₁₋₆-alkyl group,
   while the hydrogen atoms of the straight-chain or branched C₁₋₆-alkyl group may optionally be wholly or partly replaced by fluorine atoms, and may optionally be substituted by a C₁₋₅-alkyloxy group, while the hydrogen atoms of the C₁₋₅-alkyloxy group may optionally be wholly or partly replaced by fluorine atoms, or
R⁴ and R⁵ together with the carbon atom to which they are bound, form a C₃₋₈-cycloalkyl or C₃₋₈-cycloalkenyl group,
   while one of the methylene groups of a C₄₋₈-cycloalkyl group may be replaced by an oxygen atom or an —N(R^{8c})—, or a carbonyl or sulphonyl group, and/or
   two directly adjacent methylene groups of a C₄₋₈-cycloalkyl group may together be replaced by a —C(O)N(R^{8b})— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or —N(R^{8b})S(O)₂N(R^{8b})— group,
   while one or two carbon atoms of a C₃₋₈-cycloalkyl group may optionally be substituted independently of one another in each case by one or two identical or different halogen atoms or C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₄-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, or N-(C₁₋₅-alkylsulphonyl)-C₁₋₅-alkylamino groups
   while one or two carbon atoms of a C₃₋₈-cycloalkenyl group may optionally be substituted independently of one another in each case by one or two identical or different C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl groups,
   and one or two carbon atoms of a C₄₋₈-cycloalkenyl group which are not bound to another carbon atom by a double bond may optionally each be substituted independently of one another by one or two identical or different fluorine atoms or hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonyl-amino groups,
   with the proviso that a C₃₋₈-cycloalkyl or C₃₋₈-cycloalkenyl group of this kind, formed from R⁴ and R⁵ together,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a fluorine, chlorine, bromine or iodine atom, a nitrile group, a C₁₋₃-alkyl or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 4th embodiment of the present invention comprises those compounds of general formula I, wherein
A denotes a group of general formula 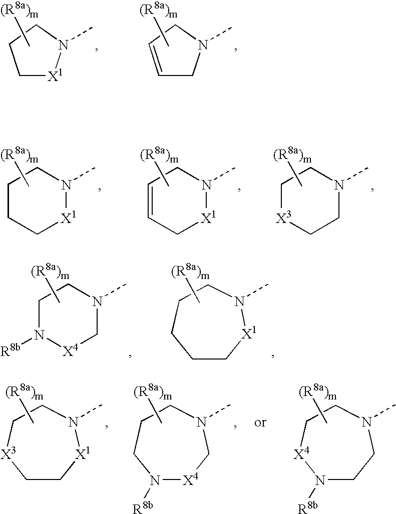
   wherein
m is the number 1 or 2,
R^{8a} each independently of one another denote a hydrogen or halogen atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylaminocarbonyl, di-(C₁₋₃-alkyl)-aminocarbonyl or C₁₋₃-alkylcarbonylamino group, while
   in the previously mentioned substituted 5- to 7-membered groups A the heteroatoms F, Cl, Br, I, O or N optionally introduced with R^{8a} as substituent are not separated by precisely one carbon atom from a heteroatom selected from among N, O, S,
R^{8b} in each case independently of one another denotes a hydrogen atom or a C₁₋₃-alkyl group,
R^{8c} in each case independently of one another denotes a hydrogen atom, a C₁₋₃-alkyl, C₁₋₃-alkylcarbonyl, C₁₋₄-alkyloxycarbonyl or C₁₋₃-alkylsulphonyl group,
X¹ denotes an oxygen atom or a —CH₂—, —CHR^{8a}— or —NR^{8c}— group,
X³ denotes an oxygen atom or a —NR^{8c}— group,
X⁴ denotes a carbonyl or sulphonyl group,
R¹ denotes a fluorine, chlorine, bromine or iodine atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms,
R² denotes a hydrogen or halogen atom or a methyl group,
R³ denotes a hydrogen atom,
R⁴ denotes a hydrogen atom, a C₂₋₄-alkenyl or C₂₋₄-alkynyl group,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a nitrile, hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl-C₁₋₃-alkyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, C₁₋₃-alkylsulphonylamino, or N-(C₁₋₃-alkylsulphonyl)-C₁₋₃-alkylamino group,
   a phenyl, heteroaryl, phenyl-C₁₋₃-alkyl or heteroaryl-C₁₋₃-alkyl group,
   which may optionally be mono- to trisubstituted in the phenyl or heteroaryl moiety by identical or different substituents selected from among halogen atoms, C₁₋₃-alkyl, di-(C₁₋₃-alkyl)-amino, hydroxy, C₁₋₃-alkyloxy, mono-, di- or trifluoromethoxy, carboxy- and C₁₋₃-alkyloxycarbonyl groups,
R⁵ denotes a hydrogen atom, a C₂₋₄-alkenyl or C₂₋₄-alkynyl group,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl or C₃₋₈-cycloalkenyl group,
   while one of the methylene groups of a C₄₋₈-cycloalkyl group may be replaced by an oxygen atom or an —N(R^{8c})— group, and/or
   two directly adjacent methylene groups of a C₄₋₈-cycloalkyl group may together be replaced by a —C(O)N(R^{8b})— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or
   —N(R^{8b})S(O)₂N(R^{8b})— group,
   while one or two carbon atoms of a C₃₋₈-cycloalkyl group may optionally be substituted independently of one another in each case by a C₁₋₃-alkyl, hydroxy, C₁₋₃-alkyloxy, C₁₋₃-alkylcarbonyloxy, C₁₋₃-alkyloxycarbonyl, amino, C₁₋₃-alkylamino, di-(C₁₋₃-alkyl)-amino, C₁₋₃-alkylcarbonylamino or C₁₋₃-alkylsulphonylamino group,
   while one or two carbon atoms of a C₃₋₈-cycloalkenyl group may optionally be substituted independently of one another in each case by one or two identical or different C₁₋₃-alkyl groups,
   and one or two carbon atoms of a C₄₋₈-cycloalkenyl group which are not bound to another carbon atom by a double bond may optionally be substituted independently of one another by one or two identical or different hydroxy, C₁₋₃-alkyloxy or di-(C₁₋₃-alkyl)-amino group,
   with the proviso that a C₃₋₈-cycloalkyl or C₃₋₈-cycloalkenyl group of this kind, formed from R⁴ and R⁵ together,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a fluorine, chlorine, bromine or iodine atom, a methyl group, or a methoxy group, while the hydrogen atoms of the methyl or methoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 5th embodiment of the present invention comprises those compounds of general formula I, wherein
A denotes a group of general formula 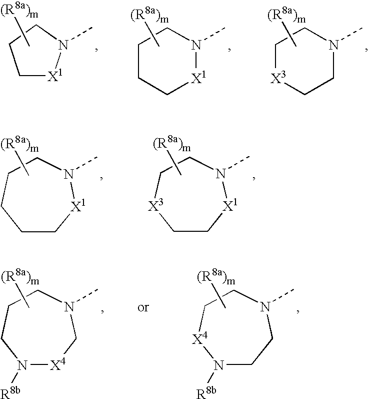
   wherein
m is the number 1 or 2,
R^{8a} in each case independently of one another denotes a hydrogen or halogen atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl or di-(C₁₋₃-alkyl)-amino-C₁₋₃-alkyl group, while in the previously mentioned substituted 5- to 7-membered groups A the heteroatoms F, Cl, Br, I, O or N optionally introduced with R^{8a} as substituents are not separated by precisely one carbon atom from a heteroatom selected from among N, O, S,
R^{8b} in each case independently of one another denotes a hydrogen atom or a C₁₋₃-alkyl group,
R^{8c} in each case independently of one another denotes a hydrogen atom, a C₁₋₃-alkyl, C₁₋₃-alkylcarbonyl or C₁₋₄-alkyloxycarbonyl group,
X¹ denotes an oxygen atom or a —CH₂—, —CHR^{8a}— or —NR^{8c}— group,
X³ denotes an oxygen atom or a —NR^{8c}— group,
X⁴ denotes a carbonyl group,
R¹ denotes a fluorine, chlorine, bromine or iodine atom, a methyl or methoxy group, while the hydrogen atoms of the methyl or methoxy group may optionally be wholly or partly replaced by fluorine atoms,
R² denotes a hydrogen or fluorine atom,
R³ denotes a hydrogen atom,
R⁴ denotes a hydrogen atom,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
   a phenyl, heteroaryl, phenyl-C₁₋₃-alkyl or heteroaryl-C₁₋₃-alkyl group,
      which may optionally be mono- to trisubstituted in the phenyl or heteroaryl moiety by identical or different substituents selected from among halogen atoms, C₁₋₃-alkyl, di-(C₁₋₃-alkyl)-amino, hydroxy, C₁₋₃-alkyloxy, mono-, di- or trifluoromethoxy groups,
R⁵ denotes a hydrogen atom,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl group,
   while one of the methylene groups of a C₄₋₈-cycloalkyl group may be replaced by an oxygen atom or a —N(R^{8c})— group, and/or
   two directly adjacent methylene groups of a C₄₋₈-cycloalkyl group may together be replaced by a —C(O)N(R^{8b})— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or
   —N(R^{8b})S(O)₂N(R^{8b})— group,
   while one or two carbon atoms of a C₃₋₈-cycloalkyl group may optionally be substituted independently of one another in each case by a C₁₋₃-alkyl, hydroxy, C₁₋₃-alkyloxy, C₁₋₃-alkylcarbonyloxy, C₁₋₃-alkyloxycarbonyl, C₁₋₃-alkylamino, di-(C₁₋₃-alkyl)-amino, C₁₋₃-alkylcarbonylamino or C₁₋₃-alkylsulphonylamino group,
   with the proviso that a C₃₋₈-cycloalkyl group of this kind, formed from R⁴ and R⁵ together,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a fluorine, chlorine, bromine or iodine atom, a methyl group, or a methoxy group, while the hydrogen atoms of the methyl or methoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an optionally by a C₁₋₃-alkyl, phenyl or phenyl-C₁₋₃-alkyl group substituted imino group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 6th embodiment of the present invention comprises those compounds of general formula I, wherein
A denotes a group of general formula 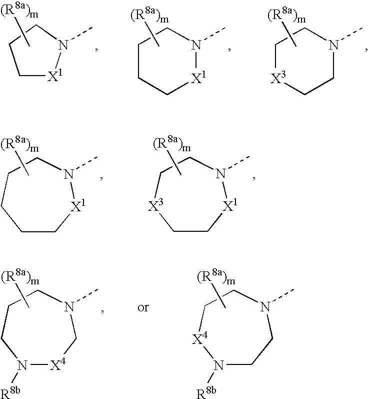
   wherein
m is the number 1 or 2,
R^{8a} in each case independently of one another denotes a hydrogen or halogen atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, or di-(C₁₋₃-alkyl)-amino-C₁₋₃-alkyl group, while in the previously mentioned substituted 5- to 7-membered groups A the heteroatoms F, Cl, Br, I, O or N optionally introduced with R^{8a} as substituents are not separated by precisely one carbon atom from a heteroatom selected from among N, O, S,
R^{8c} in each case independently of one another denotes a hydrogen atom, a C₁₋₃-alkyl, C₁₋₃-alkylcarbonyl, or C₁₋₄-alkyloxycarbonyl,
X¹ denotes an oxygen atom or a —CH₂—, —CHR^{8a}— or —NR^{8c}— group,
X³ denotes an oxygen atom or a —NR^{8c}— group,
X⁴ denotes a carbonyl group,
R¹ denotes a fluorine, chlorine, bromine or iodine atom, a methyl or methoxy group, while the hydrogen atoms of the methyl or methoxy group may optionally be wholly or partly replaced by fluorine atoms,
R² denotes a hydrogen or fluorine atom,
R³ denotes a hydrogen atom,
R⁴ denotes a hydrogen atom,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₁₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
   a phenyl, heteroaryl, phenyl-C₁₋₃-alkyl or heteroaryl-C₁₋₃-alkyl group,
      which may optionally be mono- to trisubstituted in the phenyl or heteroaryl moiety by identical or different substituents selected from the group consisting of halogen atoms, C₁₋₃-alkyl, di-(C₁₋₃-alkyl)-amino, hydroxy, C₁₋₃-alkyloxy, mono-, di- or trifluoromethoxy groups,
R⁵ denotes a hydrogen atom,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl group,
   while one of the methylene groups of a C₄₋₈-cycloalkyl group may be replaced by an oxygen atom or a —N(R^{8c})— group,
   while one or two carbon atoms of a C₃₋₈-cycloalkyl group may optionally be substituted independently of one another in each case by a C₁₋₃-alkyl, hydroxy, C₁₋₃-alkyloxy, or di-(C₁₋₃-alkyl)-amino group,
   with the proviso that a C₃₋₈-cycloalkyl group of this kind, formed together from R⁴ and R⁵,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly,
   is excluded,
R⁶ denotes a fluorine, chlorine, bromine or iodine atom,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, or an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group, or an oxygen or sulphur atom and additionally a nitrogen atom or
   an imino group optionally substituted by a C₁₋₃-alkyl or phenyl-C₁₋₃-alkyl group, and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while unless otherwise stated the alkyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 7th embodiment of the present invention comprises those compounds of general formula I, wherein
A denotes a group of general formula 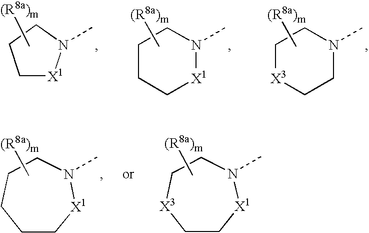
   wherein
m is the number 1 or 2,
R^{8a} in each case independently of one another denotes a hydrogen or halogen atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, or di-(C₁₋₃-alkyl)-amino-C₁₋₃-alkyl group, while in the previously mentioned substituted 5- to 7-membered groups A the heteroatoms F. Cl, Br, I, O or N optionally introduced with R^{8a} as substituents are not separated by precisely one carbon atom from a heteroatom selected from among N, O, S,
R^{8c} in each case independently of one another denotes a hydrogen atom, a C₁₋₃-alkyl, C₁₋₃-alkylcarbonyl, or C₁₋₄-alkyloxycarbonyl group,
X¹ denotes an oxygen atom or a —CH₂—, —CHR^{8a}— or —NR^{8c}— group,
X³ denotes an oxygen atom or a —NR^{8c}— group,
R¹ denotes a fluorine, chlorine or bromine atom, a methyl, trifluoromethyl or methoxy group,
R² denotes a hydrogen or fluorine atom,
R³ denotes a hydrogen atom,
R⁴ denotes a straight-chain or branched C₁₋₄-alkyl group,
   while the hydrogen atoms of the straight-chain or branched C₁₋₄-alkyl group may optionally be wholly or partly replaced by fluorine atoms, and may optionally be substituted by a hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
   a phenyl, heteroaryl, phenyl-C₁₋₃-alkyl or heteroaryl-C₁₋₃-alkyl group,
      which may optionally be mono- to trisubstituted in the phenyl or heteroaryl moiety by identical or different substituents selected from the group consisting of halogen atoms, C₁₋₃-alkyl, di-(C₁₋₃-alkyl)-amino, hydroxy, C₁₋₃-alkyloxy, mono-, di- or trifluoromethoxy groups,
R⁵ denotes a hydrogen atom, or
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl group,
   while one of the methylene groups of a C₄₋₈-cycloalkyl group may be replaced by an oxygen atom or a —N(R^{8c})— group,
   while one or two carbon atoms of a C₃₋₈-cycloalkyl group may optionally be substituted independently of one another in each case by a C₁₋₃-alkyl, hydroxy, C₁₋₃-alkyloxy or di-(C₁₋₃-alkyl)-amino group,
   with the proviso that a C₃₋₈-cycloalkyl group of this kind formed from R⁴ and R⁵ together,
      wherein two heteroatoms in the cyclic group selected from among oxygen and nitrogen are separated from one another by precisely one optionally substituted —CH₂— group, and/or
      wherein one or both methylene groups of the cyclic group which are linked directly to the carbon atom to which the groups R⁴ and R⁵ are bound are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are directly joined together,
   is excluded,
R⁶ denotes a chlorine or bromine atom,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, or an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group, or an oxygen or sulphur atom and additionally a nitrogen atom or
   an imino group optionally substituted by a C₁₋₃-alkyl or phenyl-C₁₋₃-alkyl group, and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while the alkyl and alkoxy groups contained in the foregoing definitions which have more than two carbon atoms, unless otherwise stated, may be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

An 8th embodiment of the present invention comprises those compounds of general formula I corresponding to the embodiments 1, 2, 3, 4, 5, 6 and 7 wherein R⁴ and R⁵ does not represent hydrogen.

A 9th embodiment of the present invention comprises those compounds of general formula I corresponding to the embodiments 1, 2, 3, 4, 5, 6, 7 and 8 wherein R⁶ denotes a bromine atom.

A 10th embodiment of the present invention comprises those compounds of general formula I corresponding to the embodiments 1, 2, 3, 4, 5, 6, 7, 8 and 9 wherein R⁴ and R⁵ does not represent hydrogen and R⁶ denotes a bromine atom.

An 11th embodiment of the present invention comprises those compounds of general formula I, wherein
A denotes a group of general formula 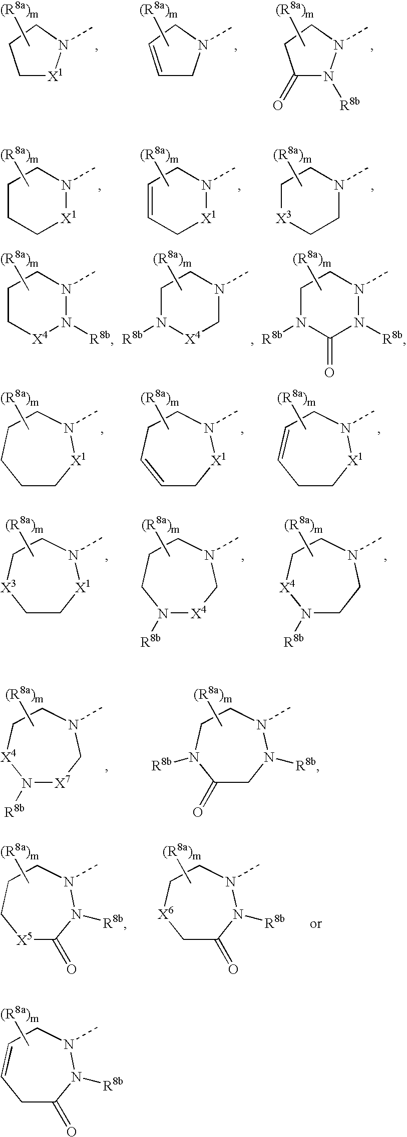
   wherein
m is the number 1 or 2,
R^{8a} in each case independently of one another denotes a hydrogen or fluorine atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₅-alkyl group,
X¹ denotes an oxygen atom or a —CH₂—, —CHR^{8a}— or —NR^{8c}— group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X² denotes an oxygen atom or a —NR^{8b} group,
X³ denotes an oxygen or sulphur atom, a —NR^{8c}— group,
X⁴ denotes a carbonyl or sulphonyl group,
X⁵ denotes an oxygen atom, a —NR^{8b}— or methylene group,
X⁶ denotes an oxygen or sulphur atom or a —NR^{8c}— group,
X⁷ denotes a methylene or carbonyl group,
R¹ denotes a hydrogen, fluorine, chlorine, bromine or iodine atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R³ denotes a hydrogen atom or a C₁₋₃-alkyl group,
R⁴ and R⁵ in each case independently of one another denote
   a hydrogen atom, a C₂₋₆-alkenyl or C₂₋₄-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a C₃₋₅-cycloalkyl group, a nitrile, hydroxy, a C₁₋₅-alkyloxy group, while the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino group,
   a carboxy, aminocarbonyl, C₁₋₅-alkylaminocarbonyl, C₃₋₆-cycloalkylaminocarbonyl, di-(C₁₋₅-alkyl)-aminocarbonyl, C₁₋₅-alkoxycarbonyl, C₄₋₆-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from the group consisting of halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      wherein in 4- to 7-membered cyclic groups in the cyclic moiety a methylene group may optionally be replaced by a —N(R^{8c})— group, an oxygen or sulphur atom or a —S(O) or —S(O)₂ group, or
      wherein in 4- to 7-membered cyclic groups in the cyclic moiety two adjacent methylene groups together may optionally be replaced by a —C(O)N(R^{8b}) or —S(O)₂N(R^{8b})— group, or
      wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group,
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group, is excluded,
      while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group,
   while a C₃₋₈-cycloalkyl or C₄₋₈-cycloalkenyl group may be substituted at an individual carbon atom by a C₂₋₅-alkylene group or simultaneously at two different carbon atoms by a C₁₋₄-alkylene group forming a corresponding spirocyclic group or a bridged bicyclic group,
   while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or a —N(R^{8c})—, or a carbonyl, sulphinyl or sulphonyl group, and/or
   two directly adjacent methylene groups of a C₄₋₈-cycloalkyl group together by a —C(O)N(R^{8b})—, —C(O)O— or —S(O)₂N(R^{8b})— group may be replaced, and/or
   three directly adjacent methylene groups of a C₆₋₈-cycloalkyl group may together be replaced by a —OC(O)N(R^{8b})—, —N(R^{8b})C(O)N(R^{8b})— or —N(R^{8b})S(O)₂N(R^{8b})— group,
   while 1 to 3 carbon atoms of a C₃₋₈-cycloalkyl group or a corresponding spirocyclic group as described above or a corresponding bridged bicyclic group may optionally be substituted independently of one another in each case by one or two fluorine atoms or one or two identical or different C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
   while 1 to 2 carbon atoms of a C₃₋₈-cycloalkenyl group may optionally be substituted independently of one another in each case by a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl groups,
   and 1 to 2 carbon atoms of a C₄₋₈-cycloalkenyl group which are not bound to another carbon atom by a double bond, may optionally be substituted independently of one another by one or two fluorine atoms or a hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
   with the proviso that a C₃₋₈-cycloalkyl or C₃₋₈-cycloalkenyl group of this kind formed from R⁴ and R⁵ together or a corresponding spirocyclic group as described above or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group which are linked directly to the carbon atom to which the groups R⁴ and R⁵ are bound are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are directly joined together, and/or
      wherein a heteroatom selected from among oxygen, nitrogen and sulphur is linked directly to a carbon atom which is linked to another carbon atom by a double bond, and/or
      which contains a cyclic group with three ring members, one or more of which correspond to an oxygen or sulphur atom or —N(R^{8c})— group,
   is excluded,
R⁶ denotes a hydrogen, fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term ‘heteroaryl group’ mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, or an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group, or an oxygen or sulphur atom and additionally a nitrogen atom or
   an imino group optionally substituted by a C₁₋₃-alkyl or phenyl-C₁₋₃-alkyl group, and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while the alkyl, alkenyl, alkynyl and alkoxy groups contained in the foregoing definitions which have more than two carbon atoms, unless otherwise stated, may be straight-chain or branched and the alkyl groups in the previously mentioned dialkylated groups, for example the dialkylamino groups, may be identical or different,
and the hydrogen atoms of the methyl or ethyl groups contained in the foregoing definitions may, unless otherwise stated, be wholly or partly replaced by fluorine atoms,
   the tautomers, the enantiomers, the diastereomers, the mixtures thereof and the salts thereof.

Examples of monocyclic heteroaryl groups are the pyridyl, N-oxy-pyridyl, pyrazolyl, pyridazinyl, pyrimidinyl, pyrazinyl, [1,2,3]triazinyl, [1,3,5]triazinyl, [1,2,4]triazinyl, pyrrolyl, imidazolyl, [1,2,4]triazolyl, [1,2,3]triazolyl, tetrazolyl, furanyl, isoxazolyl, oxazolyl, [1,2,3]oxadiazolyl, [1,2,4]oxadiazolyl, furazanyl, thiophenyl, thiazolyl, isothiazolyl, [1,2,3]thiadiazolyl, [1,2,4]thiadiazolyl or [1,2,5]thiadiazolyl group.

Examples of bicyclic heteroaryl groups are the benzimidazolyl, benzofuranyl, benzo[c]furanyl, benzothiophenyl, benzo[c]thiophenyl, benzothiazolyl, benzo[c]-isothiazolyl, benzo[d]isothiazolyl, benzoxazolyl, benzo[c]isoxazolyl, benzo[d]-isoxazolyl, benzo[1,2,5]oxadiazolyl, benzo[1,2,5]thiadiazolyl, benzo[1,2,3]thiadiazolyl, benzo[d][1,2,3]triazinyl, benzo[1,2,4]triazinyl, benzotriazolyl, cinnolinyl, quinolinyl, N-oxy-quinolinyl, isoquinolinyl, quinazolinyl, N-oxy-quinazolinyl, quinoxalinyl, phthalazinyl, indolyl, isoindolyl or 1-oxa-2,3-diaza-indenyl group.

Examples of the C₁₋₁-alkyl groups mentioned hereinbefore in the definitions are the methyl, ethyl, 1-propyl, 2-propyl, n-butyl, sec-butyl, tert-butyl, 1-pentyl, 2-pentyl, 3-pentyl, neo-pentyl, 3-methyl-2-butyl, 1-hexyl, 2-hexyl, 3-hexyl, 3-methyl-2-pentyl, 4-methyl-2-pentyl, 3-methyl-3-pentyl, 2-methyl-3-pentyl, 2,2-dimethyl-3-butyl or 2,3-dimethyl-2-butyl group.

Examples of the C₁₋₅-alkyloxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy, 3-pentyloxy or neo-pentyloxy group.

Examples of the C₂₋₆-alkenyl groups mentioned hereinbefore in the definitions are the ethenyl, 1-propen-1-yl, 2-propen-1-yl, 1-buten-1-yl, 2-buten-1-yl, 3-buten-1-yl, 1-penten-1-yl, 2-penten-1-yl, 3-penten-1-yl, 4-penten-1-yl, 1-hexen-1-yl, 2-hexen-1-yl, 3-hexen-1-yl, 4-hexen-1-yl, 5-hexen-1-yl, but-1-en-2-yl, but-2-en-2-yl, but-1-en-3-yl, 2-methyl-prop-2-en-1-yl, pent-1-en-2-yl, pent-2-en-2-yl, pent-3-en-2-yl, pent-4-en-2-yl, pent-1-en-3-yl, pent-2-en-3-yl, 2-methyl-but-1-en-1-yl, 2-methyl-but-2-en-1-yl, 2-methyl-but-3-en-1-yl, 2-ethyl-prop-2-en-1-yl, hex-1-en-2-yl, hex-2-en-2-yl, hex-3-en-2-yl, hex-4-en-2-yl, hex-5-en-2-yl, hex-1-en-3-yl, hex-2-en-3-yl, hex-3-en-3-yl, hex-4-en-3-yl, hex-5-en-3-yl, hex-1-en-4-yl, hex-2-en-4-yl, hex-3-en-4-yl, hex-4-en-4-yl, hex-5-en-4-yl, 4-methyl-pent-1-en-3-yl, 3-methyl-pent-1-en-3-yl, 2-methyl-pent-1-en-3-yl, 2,3-dimethyl-but-1-en-3-yl, 3,3-dimethyl-but-1-en-2-yl or 2-ethyl-but-1-en-3-yl group,

Examples of the C₂₋₆-alkynyl groups mentioned hereinbefore in the definitions are the ethynyl, 1-propynyl, 2-propynyl, 1-butyn-1-yl, 1-butyn-3-yl, 2-butyn-1-yl, 3-butyn-1-yl, 1-pentyn-1-yl, 1-pentyn-3-yl, 1-pentyn-4-yl, 2-pentyn-1-yl, 2-pentyn-3-yl, 3-pentyn-1-yl, 4-pentyn-1-yl, 2-methyl-1-butyn-4-yl, 3-methyl-1-butyn-1-yl, 3-methyl-1-butyn-3-yl, 1-hexyn-1-yl, 2-hexyn-1-yl, 3-hexyn-1-yl, 4-hexyn-1-yl, 5-hexyn-1-yl, 1-hexyn-3-yl, 1-hexyn-4-yl, 1-hexyn-5-yl, 2-hexyn-4-yl, 2-hexyn-5-yl, 3-hexyn-5-yl, 3-methyl-1-pentyn-3-yl, 4-methyl-1-pentyn-3-yl, 3-methyl-1-pentyn-4-yl, 4-methyl-1-pentyn-4-yl, 4-methyl-2-pentyn-4-yl, 4-methyl-2-pentyn-1-yl, 2,2-dimethyl-3-butyn-1-yl or 2-ethyl-3-butyn-1-yl group.

By a group which may be converted in vivo into a carboxy group is meant for example a carboxy group esterified with an alcohol wherein the alcoholic moiety preferably denotes a C₁₋₆-alkanol, a phenyl-C₁₋₃-alkanol, a C₃₋₉-cycloalkanol, a C₅₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol or an alcohol of formula
R⁹—CO—O—(R¹⁰CR¹¹)—OH,
wherein
R⁹ denotes a C₁₋₈-alkyl, C₅₋₇-cycloalkyl, phenyl or phenyl-C₁₋₃-alkyl group,
R¹⁰ denotes a hydrogen atom, a C₁₋₃-alkyl, C₅₋₇-cycloalkyl or phenyl group and
R¹¹ denotes a hydrogen atom or a C₁₋₃-alkyl group.

Preferred groups which may be cleaved from a carboxy group in vivo include a C₁₋₆-alkoxy group such as the methoxy, ethoxy, n-propyloxy, isopropyloxy, n-butyloxy, n-pentyloxy, n-hexyloxy or cyclohexyloxy group or a phenyl-C₁₋₃-alkoxy group such as the benzyloxy group.

By a group which may be converted in vivo into a hydroxyl group is meant for example a hydroxyl group esterified with a carboxylic acid wherein the carboxylic acid moiety is preferably a C₁₋₇-alkanoic acid, a phenyl-C₁₋₃-alkanoic acid, a C₃₋₉-cycloalkylcarboxylic acid, a C₅₋₇-cycloalkenecarboxylic acid, a C₃₋₇-alkenoic acid, a phenyl-C₃₋₅-alkenoic acid, a C₃₋₇-alkynoic acid or phenyl-C₃₋₅-alkynoic acid, while individual methylene groups of the carboxylic acid group may be replaced by oxygen atoms, with the proviso that no bond to the oxygen atom starts from a carbon atom which carries a double or triple bond.

Examples of preferred groups which may be cleaved in vivo from a hydroxyl group include a C₁₋₇-acyl group such as the formyl, acetyl, n-propionyl, isopropionyl, n-propanoyl, n-butanoyl, n-pentanoyl, n-hexanoyl or cyclohexylcarbonyl group or a benzoyl group as well as also a methoxyacetyl, 1-methoxypropionyl, 2-methoxypropionyl or 2-methoxy-ethoxyacetyl group.

The compounds of general formula I, wherein A, R⁴ and/or R⁵ contains a group which may be converted in vivo into a carboxy or hydroxyl group are prodrugs for those compounds of general formula I wherein A, R⁴ and/or R⁵ contains a carboxy or hydroxyl group.

A 12th embodiment of the present invention comprises those compounds of general formula I, wherein
A denotes a group of general formula 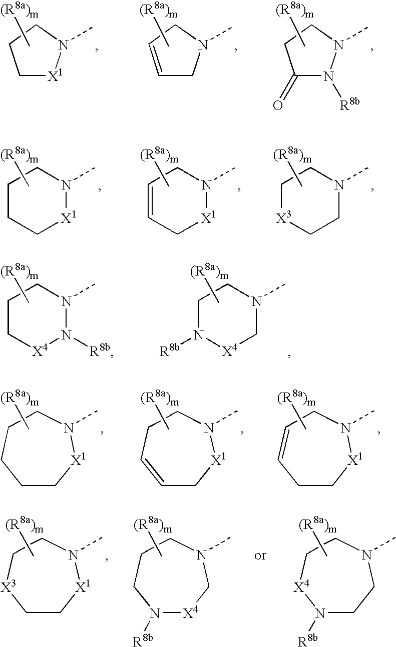
   wherein
m is the number 1 or 2,
R^{8a} in each case independently of one another denotes a hydrogen or fluorine atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₅-alkyl group,
X¹ denotes an oxygen atom or a —CH₂, —CHR^{8a}— or —NR^{8c}— group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X³ denotes an oxygen or sulphur atom, a —NR^{8c}— group,
X⁴ denotes a carbonyl or sulphonyl group,
R¹ denotes a halogen atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R³ denotes a hydrogen atom or a C₁₋₃-alkyl group,
R⁴ and R⁵ in each case independently of one another denote
   a hydrogen atom, a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a C₃₋₅-cycloalkyl group, a nitrile, hydroxy, a C₁₋₅-alkyloxy group, while the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino group,
   a carboxy, aminocarbonyl, C₁₋₅-alkylaminocarbonyl, C₃₋₆-cycloalkylaminocarbonyl, di-(C₁₋₅-alkyl)-aminocarbonyl, C₁₋₅-alkoxycarbonyl, C₄₋₆-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from the group consisting of halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      wherein at 4- to 7-membered cyclic groups in the cyclic moiety a methylene group may optionally be replaced by a —N(R^{8c})— group, an oxygen or sulphur atom or a —S(O)— or —S(O)₂— group, or
      wherein in 4- to 7-membered cyclic groups in the cyclic moiety two adjacent methylene groups together may optionally be replaced by a —C(O)N(R^{8b})— or —S(O)₂N(R^{8b})— group, or
      wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or —N(R^{8b})S(O)₂N(R^{8b})— group
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group is excluded,
      while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group,
   while a C₃₋₈-cycloalkyl or C₄₋₈-cycloalkenyl group may be substituted at an individual carbon atom by a C₂₋₅-alkylene group or simultaneously at two different carbon atoms by a C₁₋₄-alkylene group, forming a corresponding spirocyclic group or a bridged bicyclic group,
   while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or a —N(R^{8c})—, or a carbonyl, sulphinyl or sulphonyl group, and/or
   two directly adjacent methylene groups of a C₄₋₈-cycloalkyl group may together be replaced by a —C(O)N(R^{8b})—, —C(O)O or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or —N(R^{8b})S(O)₂N(R^{8b})— group,
   while 1 to 3 carbon atoms of a C₃₋₈-cycloalkyl group or a corresponding spirocyclic group as described above or a corresponding bridged bicyclic group may optionally be substituted independently of one another by in each case one or two fluorine atoms or one or two identical or different C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
   while 1 to 2 carbon atoms of a C₃₋₈-cycloalkenyl group may optionally be substituted independently of one another by a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl groups,
   and 1 to 2 carbon atoms of a C₄₋₈-cycloalkenyl group which are not bound to another carbon atom by a double bond may optionally be substituted independently of one another by one or two fluorine atoms or a hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₄-cycloalkylcarbonylamino groups,
   with the proviso that a C₃₋₈-cycloalkyl or C₃₋₈-cycloalkenyl group of this kind formed from R⁴ and R⁵ together or a corresponding spirocyclic group as described above or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly, and/or
      wherein a heteroatom selected from among oxygen, nitrogen and sulphur is linked directly to a carbon atom, which is linked to another carbon atom by a double bond, and/or
      which contains a cyclic group with three ring members, of which one or more corresponds to an oxygen or sulphur atom or N(R^{8c})— group,
   is excluded,
R⁶ denotes a fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or a C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
   the tautomers, the enantiomers, the diastereomers, the mixtures thereof and the salts thereof.

A 13th embodiment of the present invention comprises those compounds of general formula I, wherein
A denotes a group of general formula 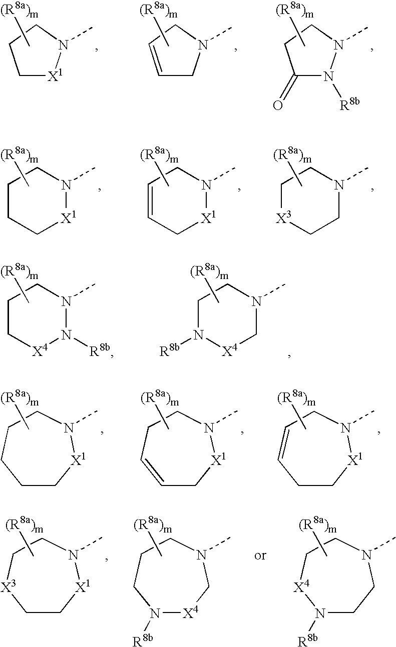
   wherein
m is the number 1 or 2,
R^{8a} in each case independently of one another denotes a hydrogen or fluorine atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
   in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₅-alkyl group,
X¹ denotes an oxygen atom or a —CH₂—, —CHR^{8a}— or —NR^{8c}— group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X³ denotes an oxygen atom, or a —NR^{8c}— group,
X⁴ denotes a carbonyl or sulphonyl group,
R¹ denotes a fluorine, chlorine, bromine or iodine atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, or a nitrile group,
R² denotes a hydrogen or halogen atom or a methyl group,
R³ denotes a hydrogen atom or a methyl group,
R⁴ denotes a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a C₃₋₅-cycloalkyl group, a nitrile, hydroxy, a C₁₋₅-alkyloxy group, while the hydrogen atoms of the C₁₋₅-alkyloxy group may optionally be wholly or partly replaced by fluorine atoms, a benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, or an N-(C₁₋₅-alkylsulphonyl)-C₁₋₅-alkylamino group,
   a carboxy, aminocarbonyl, C₁₋₅-alkylaminocarbonyl, C₃₋₆-cycloalkylaminocarbonyl, di-(C₁₋₅-alkyl)-aminocarbonyl, C₁₋₅-alkoxycarbonyl, C₄₋₄-cycloalkyleneiminocarbonyl group,
   a phenyl, heteroaryl, phenyl-C₁₋₅-alkyl or heteroaryl-C₁₋₅-alkyl group,
      which may optionally be mono- to trisubstituted in the phenyl or heteroaryl moiety by identical or different substituents selected from the group consisting of halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
   a 3- to 7-membered cycloalkyl, cycloalkyleneimino, cycloalkyl-C₁₋₅-alkyl or cycloalkyleneimino-C₁₋₃-alkyl group,
      wherein in 4- to 7-membered cyclic groups in the cyclic moiety a methylene group may optionally be replaced by a —N(R^{8c})— group, an oxygen or sulphur atom or a —S(O) or —S(O)₂ group, or
      in 4- to 7-membered cyclic groups in the cyclic moiety two adjacent methylene groups may together optionally be replaced by a —C(O)N(R^{8b})— or —S(O)₂N(R^{8b})— group, or
   wherein in 6- to 7-membered cyclic groups in the cyclic moiety three adjacent methylene groups may together optionally be replaced by a substituted —OC(O)N(R^{8b})— or —N(R^{8b})C(O)N(R^{8b}) or —N(R^{8b})S(O)₂N(R^{8b})— group,
   with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group, is excluded,
   while a 3- to 7-membered cycloalkyl, cycloalkyleneimino, cycloalkyl-C₁₋₅-alkyl or cycloalkyleneimino-C₁₋₃-alkyl group as hereinbefore defined at one or two —CH₂— groups may be substituted by in each case one or two C₁₋₃-alkyl groups,
R⁵ denotes a hydrogen atom, a C₂₋₆-alkenyl or C₂₋₆-alkynyl group,
   a straight-chain or branched C₁₋₆-alkyl group,
      while the hydrogen atoms of the straight-chain or branched C₁₋₆-alkyl group may optionally be wholly or partly replaced by fluorine atoms, and may optionally be substituted by a C₁₋₅-alkyloxy group, while the hydrogen atoms of the C₁₋₅-alkyloxy group may optionally be wholly or partly replaced by fluorine atoms, or
R⁴ and R⁵ together with the carbon atom to which they are bound form a C₃₋₈-cycloalkyl or C₃₋₈-cycloalkenyl group,
   while the C₃₋₈-cycloalkyl or C₄₋₈-cycloalkenyl group may be substituted at an individual carbon atom by a C₂₋₅-alkylene group or simultaneously at two different carbon atoms by a C₁₋₄-alkylene group forming a corresponding spirocyclic group or a bridged bicyclic group,
   while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or an —N(R^{8c})—, or a carbonyl, or sulphonyl group, and/or
   two directly adjacent methylene groups of a C₄₋₈-cycloalkyl group may together be replaced by a —C(O)N(R^{8b})—, —C(O)O— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or
   —N(R^{8b})S(O)₂N(R^{8b})— group,
   while 1 to 3 carbon atoms of a C₃₋₈-cycloalkyl group or a corresponding spirocyclic group as described above or a corresponding bridged bicyclic group may optionally be substituted independently of one another by in each case one or two fluorine atoms or one or two identical or different C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, or N-(C₁₋₅-alkylsulphonyl)-C₁₋₅-alkylamino groups,
   while 1 to 2 carbon atoms of a C₃₋₈-cycloalkenyl group may optionally be substituted independently of one another by a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl group,
   and 1 to 2 carbon atoms of a C₄₋₈-cycloalkenyl group which are not bound to another carbon atom by a double bond may optionally be substituted independently of one another by one or two fluorine atoms or a hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₄-cycloalkylcarbonylamino groups,
   with the proviso that a C₃₋₈-cycloalkyl or C₃₋₈-cycloalkenyl group of this kind, formed together from R⁴ and R⁵ or a corresponding spirocyclic group as described above or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly, and/or
      wherein a heteroatom selected from among oxygen, nitrogen and sulphur is linked directly to a carbon atom, which is linked to another carbon atom by a double bond, and/or
      which contains a cyclic group with three ring members, of which one or more corresponds to an oxygen or sulphur atom or an —N(R^{8c})— group,
   is excluded,
R⁶ denotes a fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
   the tautomers, the enantiomers, the diastereomers, the mixtures thereof and the salts thereof.

A 14th embodiment of the present invention comprises those compounds of general formula I, wherein
A denotes a group of general formula 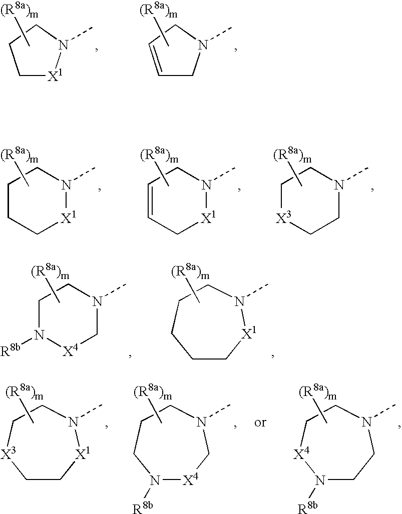
   wherein
m is the number 1 or 2,
R^{8a} in each case independently of one another denotes a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylaminocarbonyl, di-(C₁₋₃-alkyl)-aminocarbonyl or C₁₋₃-alkylcarbonylamino group, while
   in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₃-alkyl group,
X¹ denotes an oxygen atom or a —CH₂—, —CHR^{8a}— or —NR^{8c}— group,
R^{8c} in each case independently of one another denotes a hydrogen atom, a C₁₋₃-alkyl, C₁₋₃-alkylcarbonyl, C₁₋₄-alkyloxycarbonyl or C₁₋₃-alkylsulphonyl group,
X³ denotes an oxygen atom or a —NR^{8c}— group,
X⁴ denotes a carbonyl or sulphonyl group denotes,
R¹ denotes a fluorine, chlorine, bromine or iodine atom, a C₁₋₃-alkyl or a C₁₋₃-alkoxy group, while the hydrogen atoms of the C₁₋₃-alkyl or C₁₋₃-alkoxy group may optionally be wholly or partly replaced by fluorine atoms,
R² denotes a hydrogen or halogen atom or a methyl group,
R³ denotes a hydrogen atom,
R⁴ denotes a C₂₋₄-alkenyl or C₂₋₄-alkynyl group,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₃₋₅-cycloalkyl group, a nitrile, hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl-C₁₋₃-alkyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, C₁₋₃-alkylsulphonylamino, or an N-(C₁₋₃-alkylsulphonyl)-C₁₋₃-alkylamino group,
   a phenyl, heteroaryl, phenyl-C₁₋₃-alkyl or heteroaryl-C₁₋₃-alkyl group,
      which may optionally be mono- to trisubstituted in the phenyl or heteroaryl moiety by identical or different substituents selected from among halogen atoms, C₁₋₃-alkyl, di-(C₁₋₃-alkyl)-amino, hydroxy, C₁₋₃-alkyloxy, mono-, di- or trifluoromethoxy, carboxy- and C₁₋₃-alkyloxycarbonyl groups,
R⁵ denotes a hydrogen atom, a C₂₋₄-alkenyl or C₂₋₄-alkynyl group,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl or C₃₋₈-cycloalkenyl group,
   while the C₃₋₈-cycloalkyl or C₄₋₈-cycloalkenyl group may be substituted at an individual carbon atom by a C₂₋₅-alkylene group or simultaneously at two different carbon atoms by a C₁₋₄-alkylene group forming a corresponding spirocyclic group or a bridged bicyclic group,
   while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or a sulphonyl or —N(R^{8c})— group, and/or
   two directly adjacent methylene groups of a C₄₋₈-cycloalkyl group may together be replaced by a —C(O)N(R^{8b})—, —C(O)O— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or
   —N(R^{8b})S(O)₂N(R^{8b})— group,
   while 1 to 3 carbon atoms of a C₃₋₈-cycloalkyl group or a corresponding spirocyclic group as described above or a corresponding bridged bicyclic group may optionally be substituted independently of one another by a C₁₋₃-alkyl, hydroxy, C₁₋₃-alkyloxy, C₁₋₃-alkylcarbonyloxy, C₁₋₃-alkyloxycarbonyl, amino, C₁₋₃-alkylamino, di-(C₁₋₃-alkyl)-amino, C₁₋₃-alkylcarbonylamino, C₁₋₃-alkylsulphonylamino groups,
   while 1 to 2 carbon atoms of a C₃₋₈-cycloalkenyl group may optionally be substituted independently of one another by a C₁₋₃-alkyl group,
   and 1 to 2 carbon atoms of a C₄₋₈-cycloalkenyl group which are not bound to another carbon atom by a double bond may optionally be substituted independently of one another by a hydroxy, C₁₋₃-alkyloxy, di-(C₁₋₃-alkyl)-amino group,
   with the proviso that a C₃₋₈-cycloalkyl or C₃₋₈-cycloalkenyl group of this kind formed together from R⁴ and R⁵ or a corresponding spirocyclic group as described above or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly, and/or
      wherein a heteroatom selected from among oxygen, nitrogen and sulphur is linked directly to a carbon atom, which is linked to another carbon atom by a double bond, and/or
      which contains a cyclic group with three ring members, of which one or more corresponds to an oxygen or sulphur atom or an N(R^{8c})— group,
   is excluded,
R⁶ denotes a fluorine, chlorine, bromine or iodine atom, a methyl group, or a methoxy group, while the hydrogen atoms of the methyl or methoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group, an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkenyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
   the tautomers, the enantiomers, the diastereomers, the mixtures thereof and the salts thereof.

A 15th embodiment of the present invention comprises those compounds of general formula I, wherein
A denotes a group of general formula 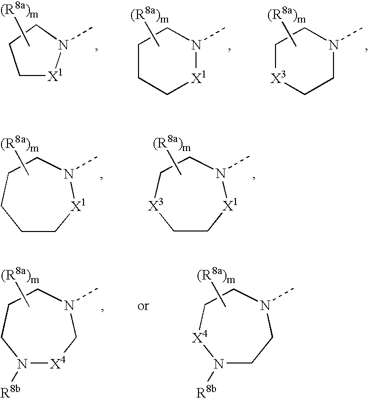
   wherein
m is the number 1 or 2,
R^{8a} in each case independently of one another denotes a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, or di-(C₁₋₃-alkyl)-amino-C₁₋₃-alkyl group, while in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₃-alkyl group,
X¹ denotes an oxygen atom or a —CH₂—, —CHR^{8a}— or —NR^{8c}— group,
R^{8c} in each case independently of one another denotes a hydrogen atom, a C₁₋₃-alkyl, C₁₋₃-alkylcarbonyl, or a C₁₋₄-alkyloxycarbonyl group,
X³ denotes an oxygen atom or a —NR^{8c}— group,
X⁴ denotes a carbonyl group,
R¹ denotes a fluorine, chlorine, bromine or iodine atom, a methyl or a methoxy group, while the hydrogen atoms of the methyl or methoxy group may optionally be wholly or partly replaced by fluorine atoms,
R² denotes a hydrogen or fluorine atom or a methyl group,
R³ denotes a hydrogen atom,
R⁴ denotes a C₂₋₄-alkenyl or C₂₋₄-alkynyl group,
   a straight-chain or branched C₁₋₄-alkyl group, while the hydrogen atoms of the straight-chain or branched C₁₋₄-alkyl group may optionally be wholly or partly replaced by fluorine atoms, and may optionally be substituted by a nitrile, hydroxy, a C₁₋₃-alkyloxy group, while the hydrogen atoms of the C₁₋₃-alkyloxy group may optionally be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
   a phenyl, heteroaryl, phenyl-C₁₋₃-alkyl or heteroaryl-C₁₋₃-alkyl group,
      which may optionally be mono- to trisubstituted in the phenyl or heteroaryl moiety by identical or different substituents selected from among halogen atoms, C₁₋₃-alkyl, di-(C₁₋₃-alkyl)-amino, hydroxy, C₁₋₃-alkyloxy, mono-, di- and trifluoromethoxy groups,
R⁵ denotes a hydrogen atom,
   a straight-chain or branched C₁₋₄-alkyl group,
      while the hydrogen atoms of the straight-chain or branched C₁₋₄-alkyl group may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₈-cycloalkyl or C₄₋₈-cycloalkenyl group,
   while the C₃₋₈-cycloalkyl or C₄₋₈-cycloalkenyl group may be substituted at an individual carbon atom by a C₂₋₅-alkylene group or may be substituted simultaneously at two different carbon atoms by a C₁₋₄-alkylene group forming a corresponding spirocyclic group or a bridged bicyclic group,
   while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or a sulphonyl or —N(R^{8c})— group, and/or
   two directly adjacent methylene groups of a C₄₋₈-cycloalkyl group may together be replaced by a —C(O)N(R^{8b})—, —C(O)O— or —S(O)₂N(R^{8b})— group, and/or
   three directly adjacent methylene groups of a C₆₋₈-cycloalkyl group may together be replaced by a —OC(O)N(R^{8b})—, —N(R^{8b})C(O)N(R^{8b})— or
   —N(R^{8b})S(O)₂N(R^{8b})— group,
   while 1 to 2 carbon atoms of a C₃₋₈-cycloalkyl group or a corresponding spirocyclic group as described above or a corresponding bridged bicyclic group may optionally be substituted independently of one another by a C₁₋₃-alkyl, hydroxy, C₁₋₃-alkyloxy, C₁₋₃-alkylcarbonyloxy, C₁₋₃-alkyloxycarbonyl, C₁₋₃-alkylamino, di-(C₁₋₃-alkyl)-amino, C₁₋₃-alkylcarbonylamino, C₁₋₃-alkylsulphonylamino groups,
   with the proviso that a C₃₋₈-cycloalkyl or C₄₋₈-cycloalkenyl group of this kind formed from R⁴ and R⁵ together or a corresponding spirocyclic group as described above or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
      wherein two oxygen atoms are joined together directly, and/or
      wherein a heteroatom selected from among oxygen, nitrogen and sulphur is linked directly to a carbon atom, which is linked to another carbon atom by a double bond, and/or
      which contains a cyclic group with three ring members, of which one or more corresponds to an oxygen or sulphur atom or an —N(R^{8c})— group,
   is excluded,
R⁶ denotes a fluorine, chlorine, bromine or iodine atom, a methyl group, or a methoxy group, while the hydrogen atoms of the methyl or methoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
   the 6-membered heteroaryl group contains one, two or three nitrogen atoms and
   the 5-membered heteroaryl group contains an imino group optionally substituted by a C₁₋₃-alkyl, phenyl or phenyl-C₁₋₃-alkyl group or an oxygen or sulphur atom or
   an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
   and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while, unless otherwise stated, the alkyl, alkynyl and alkoxy groups contained in the foregoing definitions which have more than two carbon atoms may be straight-chain or branched and the alkyl groups in the previously mentioned dialkylated groups, for example the dialkylamino groups, may be identical or different,
and, unless otherwise stated, the hydrogen atoms of the methyl or ethyl groups contained in the foregoing definitions may be wholly or partly replaced by fluorine atoms,
   the tautomers, the enantiomers, the diastereomers, the mixtures thereof and the salts thereof.

A 16th embodiment of the present invention comprises those compounds of general formula I, wherein
A denotes a group of general formula 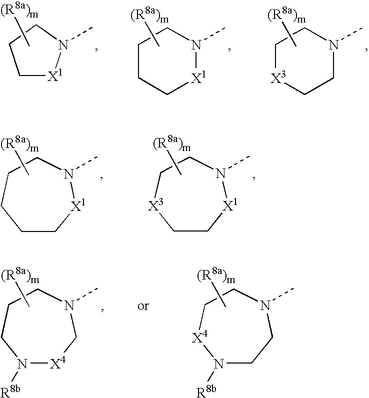
   wherein
m is the number 1 or 2,
R^{8a} in each case independently of one another denotes a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl group, while in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} denotes a hydrogen atom or a C₁₋₃-alkyl group,
X¹ denotes an oxygen atom or a —CH₂—, —CHR^{8a}— or —NR^{8c}— group,
R^{8c} in each case independently of one another denotes a hydrogen atom, a C₁₋₃-alkyl, C₁₋₃-alkylcarbonyl, or a C₁₋₄-alkyloxycarbonyl group,
X³ denotes an oxygen atom or a —NR^{8c}— group,
X⁴ denotes a carbonyl group,
R¹ denotes a fluorine, chlorine, bromine or iodine atom, a methyl or a methoxy group, while the hydrogen atoms of the methyl or methoxy group may optionally be wholly or partly replaced by fluorine atoms,
R² denotes a hydrogen or fluorine atom,
R³ denotes a hydrogen atom,
R⁴ denotes a straight-chain or branched C₁₋₄-alkyl group,
   while the hydrogen atoms may optionally be wholly or partly replaced by fluorine atoms, and may optionally be substituted by a hydroxy, a C₁₋₃-alkyloxy group, while the hydrogen atoms of the C₁₋₃-alkyloxy group may be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
   a heteroaryl-C₁₋₂-alkyl or C-linked heteroaryl group
      while the heteroaryl group is selected from among pyrrolyl, oxazolyl, imidazolyl, furanyl, thiophenyl, thiazolyl, 1,2,3-triazolyl, 1,2,4-triazolyl, tetrazolyl, pyridinyl, pyrimidinyl and pyrazinyl, and may optionally be mono- to disubstituted in the heteroaryl moiety by identical or different substituents selected from among halogen atoms, C₁₋₃-alkyl, hydroxy, C₁₋₃-alkyloxy, mono-, di- and trifluoromethoxy groups,
R⁵ denotes a hydrogen atom,
   a straight-chain or branched C₁₋₄-alkyl group,
      while the hydrogen atoms may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₇-cycloalkyl or C₄₋₇-cycloalkenyl group,
   while the C₃₋₇-cycloalkyl or C₄₋₇-cycloalkenyl group may be substituted at an individual carbon atom by a C₂₋₅-alkylene group or may be substituted simultaneously at two different carbon atoms by a C₁₋₄-alkylene group forming a corresponding spirocyclic group or a bridged bicyclic group,
   while one of the methylene groups of a C₄₋₇-cycloalkyl or C₄₋₇-cycloalkenyl group or a corresponding spirocyclic group as described above or a corresponding bridged bicyclic group may be replaced by an oxygen or sulphur atom or a sulphonyl or —N(R^{8c})— group, and/or
   two directly adjacent methylene groups of a C₄₋₈-cycloalkyl group may together be replaced by a —C(O)N(R^{8b})— or —C(O)O— group,
   while 1 to 2 carbon atoms of a C₃₋₇-cycloalkyl group or a corresponding spirocyclic group as described above or a corresponding bridged bicyclic group may optionally be substituted independently of one another by a C₁₋₃-alkyl, hydroxy, C₁₋₃-alkyloxy, di-(C₁₋₃-alkyl)-amino group,
   with the proviso that a C₃₋₇-cycloalkyl or C₄₋₇-cycloalkenyl group of this kind formed from R⁴ and R⁵ together or a corresponding spirocyclic group as described above or a corresponding bridged bicyclic group,
      wherein methylene groups of the cyclic group which are directly connected to the carbon atom to which the groups R⁴ and R⁵ are bound, are replaced by a heteroatom selected from among oxygen, nitrogen and sulphur, and/or
      wherein a substituent bound to the cyclic group, which is characterised in that a heteroatom selected from among oxygen and nitrogen is bound directly to the cyclic group, is separated from another heteroatom selected from among oxygen, nitrogen and sulphur, with the exception of the sulphone group, by precisely one, optionally substituted, methylene group, and/or
      wherein a heteroatom selected from among oxygen, nitrogen and sulphur is linked directly to a carbon atom, which is linked to another carbon atom by a double bond, and/or
      which contains a cyclic group with three ring members, of which one or more corresponds to an oxygen or sulphur atom or an —N(R^{8c})— group,
   is excluded,
R⁶ denotes a chlorine or bromine atom,
while, unless otherwise stated, by the term “halogen atom” mentioned hereinbefore in the definitions is meant an atom selected from among fluorine, chlorine, bromine and iodine,
while, unless otherwise stated, the alkyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
   the tautomers, the enantiomers, the diastereomers, the mixtures thereof and the salts thereof.

A 17th embodiment of the present invention comprises those compounds of general formula I, wherein
A denotes a group of general formula 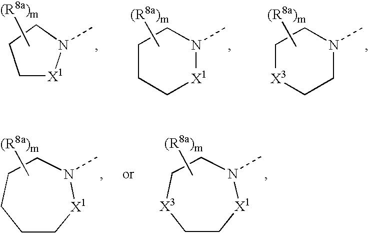
   wherein
m is the number 1 or 2,
R^{8a} each independently of one another denote a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl group, while in the previously mentioned substituted 5- to 7-membered groups A the heteroatoms F, O or N optionally introduced with R^{8a} as substituent are not separated by precisely one carbon atom from a heteroatom selected from among N, O, S,
R^{8b} denotes a hydrogen atom or a C₁₋₃-alkyl group,
X¹ denotes an oxygen atom or a —CH₂—, —CHR^{8a}— or —NR^{8c}— group,
R^{8c} denotes a hydrogen atom, a C₁₋₃-alkyl, C₁₋₃-alkylcarbonyl, or a C₁₋₄-alkyloxycarbonyl group,
X³ denotes an oxygen atom or a —NR^{8c}— group,
R¹ denotes a chlorine or bromine atom, a methyl, trifluoromethyl or a methoxy group,
R² denotes a hydrogen or fluorine atom,
R³ denotes a hydrogen atom,
R⁴ denotes a methyl group which may optionally be substituted by a hydroxy, methoxy, benzyloxy, methoxycarbonyl or pyridin-4-yl group, or
   a furan-2-yl, 1-methyl-pyrazin-3-yl, phenyl, pyridin-3-yl or pyrazin-2-yl group,
R⁵ denotes a hydrogen atom or a methyl group, or
R⁴ and R⁵ together with the carbon atom to which they are bound form a C₃₋₆-cycloalkyl or C₅₋₆-cycloalkenyl group,
   while the C₃₋₆-cycloalkyl or C₅₋₆-cycloalkenyl group may be substituted at a single carbon atom by a C₂₋₄-alkylene group or simultaneously at two different carbon atoms by a C₁₋₃-alkylene group, forming a corresponding spirocyclic group or a bridged bicyclic group,
   while one of the methylene groups of a C₄₋₆-cycloalkyl or C₅₋₆-cycloalkenyl group or of a corresponding spirocyclic group or a corresponding bridged bicyclic group as described above, may be replaced by an oxygen atom or an —N(R^{8c})— group,
   with the proviso that a C₃₋₆-cycloalkyl or C₅₋₆-cycloalkenyl group of this kind, formed from R⁴ and R⁵ together or a corresponding spirocyclic group or a corresponding bridged bicyclic group as described above,
      wherein methylene groups of the cyclic group which are directly connected to the carbon atom to which the groups R⁴ and R⁵ are bound, are replaced by a heteroatom selected from among oxygen and nitrogen, and/or
      wherein a heteroatom selected from among oxygen and nitrogen is linked directly to a carbon atom, which is linked to another carbon atom by a double bond, and/or
      which contains a cyclic group with three ring members, of which one or more corresponds to an oxygen atom or —N(R^{8c})— group,
   is excluded,
R⁶ denotes a chlorine or bromine atom
while, unless otherwise stated, by the term “halogen atom” mentioned hereinbefore in the definitions is meant an atom selected from among fluorine, chlorine, bromine and iodine,
while, unless otherwise stated, the alkyl and alkoxy groups contained in the foregoing definitions, which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
   the tautomers, the enantiomers, the diastereomers, the mixtures thereof and the salts thereof.

An 18th embodiment of the present invention comprises those compounds of general formula I corresponding to the embodiments 9, 10, 11, 12, 13, 14, 15, 16 or 17, wherein R⁴ and R⁵ does not represent hydrogen.

A 19th embodiment of the present invention comprises those compounds of general formula I corresponding to the embodiments 9, 10, 11, 12, 13, 14, 15, 16, 17 or 18, wherein R⁴ and R⁵ together with the carbon atom to which they are bound form a cyclic group, which is defined in each case as in the 9th, 10th, 11th, 12th, 13th, 14th, 15th, 16th, 17th or 18th embodiment.

A 20th embodiment of the present invention comprises those compounds of general formula I corresponding to the embodiments 9, 10, 11, 12, 13, 14, 15, 16, 17, 18 or 19, wherein R⁴ and R⁵ together with the carbon atom to which they are bound form a cyclic group, which is defined in each case as in the 9th, 10th, 11th, 12th, 13th, 14th, 15th, 16th, 17th, 18th or 19th embodiment, while in the cyclic group a methylene group is replaced by an oxygen atom or a —N(R^{8c})— group.

A 21 st embodiment of the present invention comprises those compounds of general formula I corresponding to the embodiments 9, 10, 11, 12, 13, 14, 15, 16, 17, 18, or 19, wherein R⁴ and R⁵ together with the carbon atom to which they are bound form a cyclic group which by corresponding substitution denotes a bridged bicyclic group or a spirocyclic group as described in the 9th, 10th, 11th, 12th, 13th, 14th, 15th, 16th, 17th, 18th or 19th embodiment.

A 22nd embodiment of the present invention comprises those compounds of general formula I corresponding to the embodiments 9, 10, 11, 12, 13, 14, 15, 16, 17, 18, 19 or 20, wherein R⁴ and R⁵ together with the carbon atom to which they are bound denote a cyclic group

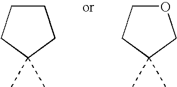

A 23rd embodiment of the present invention comprises those compounds of general formula I corresponding to the embodiments 9, 10, 11, 12, 13, 14, 15, 16, 17, 18, 19 or 20, wherein R⁴ and R⁵ together with the carbon atom to which they are bound denote a bridged bicyclic group

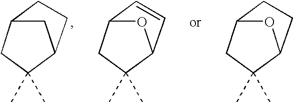

A 24th embodiment of the present invention comprises those compounds of general formula I corresponding to the embodiments 9, 10, 11, 12, 13, 14, 15, 16, 17, 18, 19, 20, 21, 22 or 23, wherein the group A denotes the group

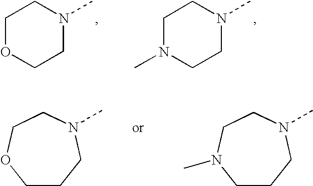

A 25th embodiment of the present invention comprises those compounds of general formula I corresponding to the embodiments 9, 10, 11, 12, 13, 14, 15, 16, 17, 18, 19, 20, 21, 22 or 23, wherein the group A denotes the group

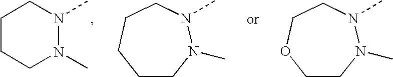

A 26th embodiment of the present invention comprises those compounds of general formula I corresponding to the embodiments 9, 10, 11, 12, 13, 14, 15, 16, 17, 18, 19, 20, 21, 22, 23, 24 or 25, wherein R⁶ denotes a bromine atom.

A 27th embodiment of the present invention comprises those compounds of general formula I corresponding to the embodiments 9, 10, 11, 12, 13, 14, 15, 16, 17, 18, 19, 20, 21, 22, 23, 24 or 25, wherein R⁶ denotes a chlorine atom.

A 28th embodiment of the present invention comprises those compounds of general formula I corresponding to the embodiments 9, 10, 11, 12, 13, 14, 15, 16, 17, 18, 19, 20, 21, 22, 23, 24, 25, 26 or 27, wherein R¹ denotes a fluorine, chlorine or bromine atom or a methyl or trifluoromethyl group.

A 29th embodiment of the present invention comprises those compounds of general formula I corresponding to the embodiments 9, 10, 11, 12, 13, 14, 15, 16, 17, 18, 19, 20, 21, 22, 23, 24, 25, 26 or 27, wherein R¹ denotes a hydrogen atom.

The following preferred compounds of general formula I will now be mentioned by way of example:
(1) 5-chloro-thiophene-2-carboxylic acid-N-{(1R)-2-benzyloxy-1-[3-chloro-4-(4-methyl-[1,4]diazepan-1-yl)-phenylcarbamoyl)-ethyl}-amide,
(2) 5-chloro-thiophene-2-carboxylic acid-N-{1-[3-bromo-4-(4-methyl-piperazin-1-yl)-phenylcarbamoyl]-ethyl}-amide,
(3) 5-chloro-thiophene-2-carboxylic acid-N-{(1R)-1-[3-chloro-4-(4-methyl-[1,4]diazepan-1-yl)-phenylcarbamoyl]-2-hydroxy-ethyl}-amide,
(4) 5-bromo-thiophene-2-carboxylic acid-N-{(1R)-2-benzyloxy-1-[3-chloro-4-(4-methyl-[1,4]diazepan-1-yl)-phenylcarbamoyl]-ethyl}-amide,
(5) 5-bromo-thiophene-2-carboxylic acid-N-{1-[3-chloro-4-(4-methyl-[1,4]diazepan-1-yl)-phenylcarbamoyl]-1-methyl-ethyl}-amide,
(6) 5-chloro-thiophene-2-carboxylic acid-N-{1-[3-chloro-4-(4-methyl-[1,4]diazepan-1-yl)-phenylcarbamoyl]-1-methyl-ethyl}-amide,
(7) 5-bromo-thiophene-2-carboxylic acid-N-{1-[3-chloro-4-(morpholin-4-yl)-phenylcarbamoyl]-1-methyl-ethyl}-amide,
(8) 5-chloro-thiophene-2-carboxylic acid-N-{1-[3-methyl-4-(morpholin-4-yl)-phenylcarbamoyl]-1-methyl-ethyl}-amide,
(9) 5-bromo-thiophene-2-carboxylic acid-N-{(1R)-1-[3-chloro-4-(morpholin-4-yl)-phenylcarbamoyl]-2-methoxy-ethyl}-amide,
(10) 5-bromo-thiophene-2-carboxylic acid-N-{1-[3-chloro-4-(pyrazolidin-1-yl)-phenylcarbamoyl]-1-methyl-ethyl)-amide,
(11) 5-chloro-thiophene-2-carboxylic acid-N-{1-[3-methyl-4-(pyrazolidin-1-yl)-phenylcarbamoyl]-1-methyl-ethyl}-amide,
(12) 5-bromo-thiophene-2-carboxylic acid-N-(1R)-1-[3-chloro-4-(pyrazolidin-1-yl)-phenylcarbamoyl]-2-methoxy-ethyl}-amide,
(13) 5-bromo-thiophene-2-carboxylic acid-N-{1-[3-chloro-4-(tetrahydropyridazin-1-yl)-phenylcarbamoyl]-1-methyl-ethyl}-amide,
(14) 5-chloro-thiophene-2-carboxylic acid-N-{1-[3-methyl-4-(tetrahydropyridazin-1-yl)-phenylcarbamoyl]-1-methyl-ethyl}-amide,
(15) 5-bromo-thiophene-2-carboxylic acid-N-{(1R)-1-[3-chloro-4-(tetrahydropyridazin-1-yl)-phenylcarbamoyl]-2-methoxy-ethyl}-amide,
(16) 5-bromo-thiophene-2-carboxylic acid-N-{1-[3-chloro-4-(2-methyl-tetrahydropyridazin-1-yl)-phenylcarbamoyl]-1-methyl-ethyl}-amide,
(17) 5-chloro-thiophene-2-carboxylic acid-N-{1-[3-methyl-4-(2-methyl-tetrahydropyridazin-1-yl)-phenylcarbamoyl]-1-methyl-ethyl}-amide,
(18) 5-bromo-thiophene-2-carboxylic acid-N-{(1R)-1-[3-chloro-4-(2-methyl-tetrahydropyridazin-1-yl)-phenylcarbamoyl]-2-methoxy-ethyl}-amide,
(19) 5-bromo-thiophene-2-carboxylic acid-N-{1-[3-chloro-4-([1,2]oxazinan-2-yl)-phenylcarbamoyl]-1-methyl-ethyl}-amide,
(20) 5-chloro-thiophene-2-carboxylic acid-N-{1-[3-methyl-4-([1,2]oxazinan-2-yl)-phenylcarbamoyl]-1-methyl-ethyl}-amide,
(21) 5-bromo-thiophene-2-carboxylic acid-N-{(1R)-1-[3-chloro-4-([1,2]oxazinan-2-yl)-phenylcarbamoyl]-2-methoxy-ethyl}-amide,
(22) 5-bromo-thiophene-2-carboxylic acid-N-{1-[3-chloro-4-([1,2]oxazepan-2-yl)-phenylcarbamoyl]-1-methyl-ethyl}-amide,
(23) 5-chloro-thiophene-2-carboxylic acid-N-{1-[3-methyl-4-([1,2]oxazepan-2-yl)-phenylcarbamoyl]-1-methyl-ethyl}-amide,
(24) 5-bromo-thiophene-2-carboxylic acid-N-{(1R)-1-[3-chloro-4-([1,2]oxazepan-2-yl)-phenylcarbamoyl]-2-methoxy-ethyl}-amide,
(25) 5-bromo-thiophene-2-carboxylic acid-N-{1-[3-chloro-4-(2-oxo-piperazin-4-yl)-phenylcarbamoyl]-ethyl}-amide,
(26) 5-bromo-thiophene-2-carboxylic acid-N-{1-[3-chloro-4-(tetrahydro-pyridazin-3-on-1-yl)-phenylcarbamoyl]-ethyl}-amide,
(27) 5-bromo-thiophene-2-carboxylic acid-N-1-[3-bromo-4-(4-methyl-[1,4]diazepan-1-yl)-phenylcarbamoyl]-1-methyl-ethyl)amide,
(28) 5-bromo-thiophene-2-carboxylic acid-N-{1-[3-trifluoromethyl-4-(4-methyl-[1,4]diazepan-1-yl)-phenylcarbamoyl]-1-methyl-ethyl}-amide,
(29) 5-bromo-thiophene-2-carboxylic acid-N-{1-[3-trifluoromethoxy-4-(4-methyl-[1,4]diazepan-1-yl)-phenylcarbamoyl]-1-methyl-ethyl}-amide,
(30) 5-chloro-thiophene-2-carboxylic acid-N-{1-[3-trifluoromethyl-4-(4-methyl-[1,4]diazepan-1-yl)-phenylcarbamoyl]-1-methyl-ethyl}-amide,
(31) 5-chloro-thiophene-2-carboxylic acid-N-{(1R)-1-[3-methyl-4-(4-methyl-[1,4]diazepan-1-yl)-phenylcarbamoyl]-3-methylsulphanyl-propyl}-amide,
(32) 5-chloro-thiophene-2-carboxylic acid-N-{1-[3-methyl-4-(4-methyl-[1,4]diazepan-1-yl)-phenylcarbamoyl]-1-phenyl-methyl}-amide,
(33) 5-chloro-thiophene-2-carboxylic acid-N-{1-[3-methyl-4-(4-methyl-[1,4]diazepan-1-yl)-phenylcarbamoyl]-1-[pyridin-3-yl]-methyl}-amide,
(34) 5-chloro-thiophene-2-carboxylic acid-N-{1-[3-methyl-4-(4-methyl-[1,4]diazepan-1-yl)-phenylcarbamoyl]-2-[imidazol-4-yl]-ethyl}-amide,
(35) 5-chloro-thiophene-2-carboxylic acid-N-{1-[3-methyl-4-(4-methyl-[1,4]diazepan-1-yl)-phenylcarbamoyl]-3-(dimethylaminocarbonyl)-propyl}-amide,
(36) 5-bromo-thiophene-2-carboxylic acid-N-{1-methyl-[3-methyl-4-(2,5-dimethyl-pyrrolidin-1-yl)-phenylcarbamoyl]-ethyl}-amide,
(37) 5-bromo-thiophene-2-carboxylic acid-N-{1-[3-methyl-4-(2,6-dimethyl-piperidin-1-yl)-phenylcarbamoyl]-cyclopentyl}-amide,
(38) 5-bromo-thiophene-2-carboxylic acid-N-{4-[3-methyl-4-(2,6-dimethyl-piperidin-1-yl)-phenylcarbamoyl]-tetrahydropyran-4-yl}-amide,
(39) 5-bromo-thiophene-2-carboxylic acid-N-{1-methyl-4-[3-methyl-4-(2,6-dimethyl-morpholin-4-yl)-phenylcarbamoyl]-piperazin-4-yl}-amide,
(40) 5-bromo-thiophene-2-carboxylic acid-N-{3-[3-chloro-4-(2,2-dimethyl-pyrrolidin-1-yl)-phenylcarbamoyl]-tetrahydrofuran-3-yl}-amide,
(41) 5-chloro-thiophene-2-carboxylic acid-N-{1-[3-chloro-4-(4-methyl-[1,4]diazepan-1-yl)-phenylcarbamoyl]-ethyl}-amide,
(42) 5-chloro-thiophene-2-carboxylic acid-N-(1R)-1-[3-chloro-4-(4-methyl-[1,4]diazepan-1-yl)-phenylcarbamoyl]-2-phenyl-ethyl)amide,
(43) 5-chloro-thiophene-2-carboxylic acid-N-{1-[3-chloro-4-(3-oxo-piperazin-1-yl)-phenylcarbamoyl]-1-methyl-ethyl}-amide,
(44) 5-bromo-thiophene-2-carboxylic acid-N-{1-[3-chloro-4-(3-oxo-piperazin-1-yl)-phenylcarbamoyl]-1-methyl-ethyl)amide,
(45) 5-chloro-thiophene-2-carboxylic acid-N-{1-[3-chloro-4-(morpholin-4-yl)-phenylcarbamoyl]-ethyl}-amide,
(46) 5-chloro-thiophene-2-carboxylic acid-N-{1-[4-(4-methyl-[1,4]diazepan-1-yl)-phenylcarbamoyl]-1-methyl-ethyl}-amide,
(47) 5-bromo-thiophene-2-carboxylic acid-N-{1-[4-(4-methyl-[1,4]diazepan-1-yl)-phenylcarbamoyl]-1-methyl-ethyl}-amide,
(48) 5-chloro-thiophene-2-carboxylic acid-N-{4-[3-chloro-4-(4-methyl-[1,4]diazepan-1-yl)-phenylcarbamoyl]-tetrahydropyran-4-yl}-amide,
(49) 5-bromo-thiophene-2-carboxylic acid-N-{4-[3-chloro-4-(4-methyl-[1,4]diazepan-1-yl)-phenylcarbamoyl]-tetrahydropyran-4-yl}-amide,
(50) 5-chloro-thiophene-2-carboxylic acid-N-{(1R)-1-[3-chloro-4-(4-methyl-[1,4]diazepan-1-yl)-phenylcarbamoyl]-1-methyl-2-phenyl-ethyl}-amide,
(51) 5-bromo-thiophene-2-carboxylic acid-N-{(1R)-1-[3-chloro-4-(4-methyl-[1,4]diazepan-1-yl)-phenylcarbamoyl]-1-methyl-2-phenyl-ethyl}-amide,
(52) 5-bromo-thiophene-2-carboxylic acid-N-{1-[4-(2,5-dimethyl-pyrrolidin-1-yl)-phenylcarbamoyl]-1-methyl-ethyl}-amide,
(53) 5-chloro-thiophene-2-carboxylic acid-N-{1-[4-(2,5-dimethyl-pyrrolidin-1-yl)-phenylcarbamoyl]-1-methyl-ethyl}-amide,
(54) 5-bromo-thiophene-2-carboxylic acid-N-{1-[3-chloro-4-(4-methyl-[1,4]diazepan-1-yl)-phenylcarbamoyl]-cyclopent-1-yl}-amide,
(55) 5-bromo-thiophene-2-carboxylic acid-N-{1-[3-methyl-4-(4-acetyl-[1,4]diazepan-1-yl)-phenylcarbamoyl]-cyclopent-1-yl}-amide,
(56) 5-chloro-thiophene-2-carboxylic acid-N-{1-methyl-1-[4-(4-methyl-piperazin-1-yl)-phenylcarbamoyl]-ethyl}-amide,
(57) 5-chloro-thiophene-2-carboxylic acid-N-{1-[3-chloro-4-(morpholin-4-yl)-phenylcarbamoyl]-1-methyl-ethyl}-amide,
(58) 5-chloro-thiophene-2-carboxylic acid-N-{1-[4-(4-methyl-piperazin-1-yl)-phenylcarbamoyl]-ethyl}-amide,
(59) 5-bromo-thiophene-2-carboxylic acid-N-3-[3-methyl-4-(2-methyl-tetrahydro-pyridazin-1-yl)-phenylcarbamoyl]-tetrahydrofuran-3-yl}-amide,
(60) 5-chloro-thiophene-2-carboxylic acid-N{3-[3-methyl-4-(2-methyl-tetrahydro-pyridazin-1-yl)-phenylcarbamoyl]-tetrahydrofuran-3-yl}-amide,
(61) 5-chloro-thiophene-2-carboxylic acid-N-2-[4-(2-methyl-tetrahydro-pyridazin-1-yl)-phenylcarbamoyl]-bicyclo[2.2.1]hept-2-yl)amide,
(62) 5-chloro-thiophene-2-carboxylic acid-N-{3-[3-chloro-4-(2-methyl-tetrahydro-pyridazin-1-yl)-phenylcarbamoyl]-tetrahydrofuran-3-yl}-amide,
(63) 5-chloro-thiophene-2-carboxylic acid-N-{3-[3-bromo-4-(2-methyl-tetrahydro-pyridazin-1-yl)-phenylcarbamoyl]-tetrahydrofuran-3-yl}-amide,
(64) 5-chloro-thiophene-2-carboxylic acid-N-{3-[3-fluoro-4-(2-methyl-tetrahydro-pyridazin-1-yl)-phenylcarbamoyl]-tetrahydrofuran-3-yl}-amide,
(65) 5-chloro-thiophene-2-carboxylic acid-N-{3-[3-trifluoromethyl-4-(2-methyl-tetrahydro-pyridazin-1-yl)-phenylcarbamoyl]-tetrahydrofuran-3-yl}-amide,
(66) 5-chloro-thiophene-2-carboxylic acid-N-{3-[4-(2-methyl-tetrahydro-pyridazin-1-yl)-phenylcarbamoyl]-tetrahydrofuran-3-yl}-amide,
(67) 5-chloro-thiophene-2-carboxylic acid-N-{2-[3-methyl-4-(2-methyl-tetrahydro-pyridazin-1-yl)-phenylcarbamoyl]-bicyclo[2.2.1]hept-2-yl}-amide,
(68) 5-chloro-thiophene-2-carboxylic acid-N-{2-[3-methyl-4-(2-methyl-tetrahydro-pyridazin-1-yl)-phenylcarbamoyl]-7-oxa-bicyclo[2.2.1]hept-2-yl}-amide,
(69) 5-chloro-thiophene-2-carboxylic acid-N-{3-[3-methyl-4-([2-methyl-[1,2]diazepan-1-yl)-phenylcarbamoyl]-tetrahydrofuran-3-yl}-amide,
(70) 5-chloro-thiophene-2-carboxylic acid-N-{3-[3-methyl-4-(5-methyl-[1,4,5]oxadiazepan-4-yl)-phenylcarbamoyl]-tetrahydrofuran-3-yl}-amide,
(71) 5-bromo-thiophene-2-carboxylic acid-N-{3-[4-(5-methyl-[1,4,5]oxadiazepan-4-yl)-phenylcarbamoyl]-tetrahydrofuran-3-yl}-amide,
   the tautomers, the enantiomers, the diastereomers, the mixtures thereof and the salts thereof, while the compounds
(1) 5-chloro-thiophene-2-carboxylic acid-N-{(1R)-2-benzyloxy-1-[3-chloro-4-(4-methyl-[1,4]diazepan-1-yl)-phenylcarbamoyl)-ethyl}-amide,
(2) 5-chloro-thiophene-2-carboxylic acid-N-1-[3-bromo-4-(4-methyl-piperazin-1-yl)-phenylcarbamoyl]-ethyl)amide,
(3) 5-chloro-thiophene-2-carboxylic acid-N-{(1R)-1-[3-chloro-4-(4-methyl-[1,4]diazepan-1-yl)-phenylcarbamoyl]-2-hydroxy-ethyl}-amide,
(4) 5-bromo-thiophene-2-carboxylic acid-N-{(1R)-2-benzyloxy-1-[3-chloro-4-(4-methyl-[1,4]diazepan-1-yl)-phenylcarbamoyl]-ethyl}-amide,
(5) 5-bromo-thiophene-2-carboxylic acid-N-{1-[3-chloro-4-(4-methyl-[1,4]diazepan-1-yl)-phenylcarbamoyl]-1-methyl-ethyl}-amide,
(6) 5-chloro-thiophene-2-carboxylic acid-N-{1-[3-chloro-4-(4-methyl-[1,4]diazepan-1-yl)-phenylcarbamoyl]-1-methyl-ethyl}-amide,
(7) 5-bromo-thiophene-2-carboxylic acid-N-{1-[3-chloro-4-(morpholin-4-yl)-phenylcarbamoyl]-1-methyl-ethyl}-amide,
(8) 5-bromo-thiophene-2-carboxylic acid-N-{1-[3-trifluoromethyl-4-(4-methyl-[1,4]diazepan-1-yl)-phenylcarbamoyl]-1-methyl-ethyl}-amide,
(9) 5-chloro-thiophene-2-carboxylic acid-N-{1-[3-trifluoromethyl-4-(4-methyl-[1,4]diazepan-1-yl)-phenylcarbamoyl]-1-methyl-ethyl}-amide,
(10) 5-chloro-thiophene-2-carboxylic acid-N-{1-[3-chloro-4-(4-methyl-[1,4]diazepan-1-yl)-phenylcarbamoyl]-ethyl}-amide,
(11) 5-chloro-thiophene-2-carboxylic acid-N-{(1R)-1-[3-chloro-4-(4-methyl-[1,4]diazepan-1-yl)-phenylcarbamoyl]-2-phenyl-ethyl}-amide,
(12) 5-chloro-thiophene-2-carboxylic acid-N-{1-[3-chloro-4-(3-oxo-piperazin-1-yl)-phenylcarbamoyl]-1-methyl-ethyl}-amide,
(13) 5-bromo-thiophene-2-carboxylic acid-N-{1-[3-chloro-4-(3-oxo-piperazin-1-yl)-phenylcarbamoyl]-1-methyl-ethyl}-amide,
(14) 5-chloro-thiophene-2-carboxylic acid-N-{1-[3-chloro-4-(morpholin-4-yl)-phenylcarbamoyl]-ethyl}-amide,
(15) 5-chloro-thiophene-2-carboxylic acid-N-{1-[4-(4-methyl-[1,4]diazepan-1-yl)-phenylcarbamoyl]-1-methyl-ethyl}-amide,
(16) 5-bromo-thiophene-2-carboxylic acid-N-{1-[4-(4-methyl-[1,4]diazepan-1-yl)-phenylcarbamoyl]-1-methyl-ethyl}-amide,
(17) 5-chloro-thiophene-2-carboxylic acid-N-{4-[3-chloro-4-(4-methyl-[1,4]diazepan-1-yl)-phenylcarbamoyl]-tetrahydropyran-4-yl}-amide,
(18) 5-bromo-thiophene-2-carboxylic acid-N-{4-[3-chloro-4-(4-methyl-[1,4]diazepan-1-yl)-phenylcarbamoyl]-tetrahydropyran-4-yl}-amide,
(19) 5-chloro-thiophene-2-carboxylic acid-N-(1R)-1-[3-chloro-4-(4-methyl-[1,4]diazepan-1-yl)-phenylcarbamoyl]-1-methyl-2-phenyl-ethyl)amide,
(20) 5-bromo-thiophene-2-carboxylic acid-N-(1R)-1-[3-chloro-4-(4-methyl-[1,4]diazepan-1-yl)-phenylcarbamoyl]-1-methyl-2-phenyl-ethyl)amide,
(21) 5-bromo-thiophene-2-carboxylic acid-N-{1-[4-(2,5-dimethyl-pyrrolidin-1-yl)-phenylcarbamoyl]-1-methyl-ethyl}-amide,
(22) 5-chloro-thiophene-2-carboxylic acid-N-1-[4-(2,5-dimethyl-pyrrolidin-1-yl)-phenylcarbamoyl]-1-methyl-ethyl)-amide,
(23) 5-bromo-thiophene-2-carboxylic acid-N-{1-[3-chloro-4-(4-methyl-[1,4]diazepan-1-yl)-phenylcarbamoyl]-cyclopent-1-yl}-amide,
(24) 5-bromo-thiophene-2-carboxylic acid-N-{1-[3-methyl-4-(4-acetyl-[1,4]diazepan-1-yl)-phenylcarbamoyl]-cyclopent-1-yl}-amide,
(25) 5-chloro-thiophene-2-carboxylic acid-N-{1-methyl-1-[4-(4-methyl-piperazin-1-yl)-phenylcarbamoyl]-ethyl}-amide,
(26) 5-chloro-thiophene-2-carboxylic acid-N-{1-[3-chloro-4-(morpholin-4-yl)-phenylcarbamoyl]-1-methyl-ethyl}-amide,
(27) 5-chloro-thiophene-2-carboxylic acid-N-{1-[4-(4-methyl-piperazin-1-yl)-phenylcarbamoyl]-ethyl}-amide,
(28) 5-bromo-thiophene-2-carboxylic acid-N-{3-[3-methyl-4-(2-methyl-tetrahydro-pyridazin-1-yl)-phenylcarbamoyl]-tetrahydrofuran-3-yl}-amide,
(29) 5-chloro-thiophene-2-carboxylic acid-N-{3-[3-methyl-4-(2-methyl-tetrahydro-pyridazin-1-yl)-phenylcarbamoyl]-tetrahydrofuran-3-yl}-amide,
(30) 5-chloro-thiophene-2-carboxylic acid-N-{3-[4-(2-methyl-tetrahydro-pyridazin-1-yl)-phenylcarbamoyl]-tetrahydrofuran-3-yl}-amide,
(31) 5-chloro-thiophene-2-carboxylic acid-N-{2-[3-methyl-4-(2-methyl-tetrahydro-pyridazin-1-yl)-phenylcarbamoyl]-bicyclo[2.2.1]hept-2-yl}-amide,
(32) 5-chloro-thiophene-2-carboxylic acid-N-{2-[3-methyl-4-(2-methyl-tetrahydro-pyridazin-1-yl)-phenylcarbamoyl]-7-oxa-bicyclo[2.2.1]hept-2-yl}-amide,
(33) 5-chloro-thiophene-2-carboxylic acid-N-{3-[3-methyl-4-(5-methyl-[1,4,5]oxadiazepan-4-yl)-phenylcarbamoyl]-tetrahydrofuran-3-yl}-amide,
(34) 5-bromo-thiophene-2-carboxylic acid-N-{3-[4-(5-methyl-[1,4,5]oxadiazepan-4-yl)-phenylcarbamoyl]-tetrahydrofuran-3-yl}-amide,
(35) 5-bromo-thiophene-2-carboxylic acid-N-1-[3-chloro-4-(2-methyl-tetrahydropyridazin-1-yl)-phenylcarbamoyl]-1-methyl-ethyl)amide,
(36) 5-chloro-thiophene-2-carboxylic acid-N-{1-[3-methyl-4-(2-methyl-tetrahydropyridazin-1-yl)-phenylcarbamoyl]-1-methyl-ethyl}-amide,
(37) 5-bromo-thiophene-2-carboxylic acid-N-{(1R)-1-[3-chloro-4-(2-methyl-tetrahydropyridazin-1-yl)-phenylcarbamoyl]-2-methoxy-ethyl}-amide,
(38) 5-bromo-thiophene-2-carboxylic acid-N-{1-[3-chloro-4-(2-oxo-piperazin-4-yl)-phenylcarbamoyl]-ethyl}-amide,
(39) 5-bromo-thiophene-2-carboxylic acid-N-{3-[3-chloro-4-(2,2-dimethyl-pyrrolidin-1-yl)-phenylcarbamoyl]-tetrahydrofuran-3-yl}-amide,
(40) 5-chloro-thiophene-2-carboxylic acid-N-{3-[3-chloro-4-(2-methyl-tetrahydro-pyridazin-1-yl)-phenylcarbamoyl]-tetrahydrofuran-3-yl}-amide,
(41) 5-chloro-thiophene-2-carboxylic acid-N-{3-[3-bromo-4-(2-methyl-tetrahydro-pyridazin-1-yl)-phenylcarbamoyl]-tetrahydrofuran-3-yl}-amide,
(42) 5-chloro-thiophene-2-carboxylic acid-N-{3-[3-fluoro-4-(2-methyl-tetrahydro-pyridazin-1-yl)-phenylcarbamoyl]-tetrahydrofuran-3-yl}-amide,
(43) 5-chloro-thiophene-2-carboxylic acid-N-{3-[3-trifluoromethyl-4-(2-methyl-tetrahydro-pyridazin-1-yl)-phenylcarbamoyl]-tetrahydrofuran-3-yl}-amide,
(44) 5-chloro-thiophene-2-carboxylic acid-N-{3-[3-methyl-4-([2-methyl-[1,2]diazepan-1-yl)-phenylcarbamoyl]-tetrahydrofuran-3-yl}-amide,
   the tautomers, the enantiomers, the diastereomers, the mixtures thereof and the salts thereof are particularly preferred.

According to the invention the compounds of general formula I are obtained by methods known per se, for example by the following methods:
(a) In order to prepare a compound of general formula 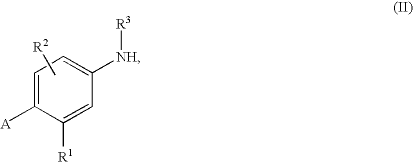
   wherein A and R¹ to R³ are as defined above:
1) Preparing a compound of general formula (II), wherein R³ denotes a hydrogen atom and A, R¹ and R² are defined as above:
i) reduction of the nitro group of a compound of general formula (III) 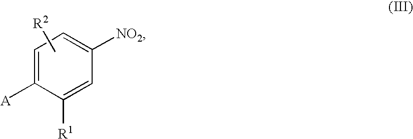
   wherein A, R¹ and R² are defined as above:

The reduction of the nitro group is for example conveniently carried out in a solvent or mixture of solvents such as water, aqueous ammonium chloride solution, hydrochloric acid, sulphuric acid, phosphoric acid, formic acid, acetic acid, acetic anhydride with base metals such as iron, zinc, tin or sulphur compounds such as ammonium sulphide, sodium sulphide or sodium dithionite or by catalytic hydrogenation with hydrogen, for example under a pressure between 0.5 and 100 bar, but preferably between 1 and 50 bar, or with hydrazine as reducing agent, conveniently in the presence of a catalyst such as for example Raney nickel, palladium charcoal, platinum oxide, platinum on mineral fibres or rhodium, or with complex hydrides such as lithium aluminium hydride, sodium borohydride, sodium cyanborohydride, diisobutylaluminium hydride, conveniently in a solvent or mixture of solvents such as water, methanol, ethanol, isopropanol, pentane, hexane, cyclohexane, heptane, benzene, toluene, xylene, ethyl acetate, methylpropionate, glycol, glycoldimethylether, diethyleneglycoldimethylether, dioxane, tetrahydrofuran, N-methylpyrrolidinone, or N-ethyl-diisopropylamine, N-C₁₋₅-alkylmorpholine, N-C₁₋₅-alkylpiperidine, N-C₁₋₅-alkylpyrrolidine, triethylamine, pyridine, for example at temperatures between −30 and 250° C., but preferably between 0 and 150° C.

The compounds of general formula (III) may be obtained as follows
a) Nucleophilic substitution with a compound of general formula
   A-H (IV),
   wherein A is defined as above, at the aromatic group of general formula 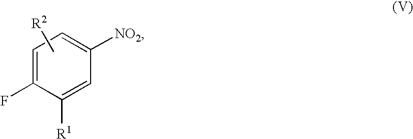
   wherein R¹ and R² are defined as above.

The nucleophilic substitution is conveniently carried out in a solvent or mixture of solvents such as ethanol, isopropanol, benzene, chlorobenzene, toluene, xylene, glycol, glycoldimethylether, diethyleneglycoldimethylether, dimethylformamide, N-methylpyrrolidinone, tetralin, dimethylsulphoxide, sulpholane, methylene chloride, chloroform, tetrachloromethane or N-ethyl-diisopropylamine, N-C₁₋₅-alkylmorpholine, N-C₁₋₅-alkylpiperidine, N-C₁₋₅-alkylpyrrolidine, triethylamine, pyridine, for example at temperatures between −30 and 250° C., but preferably between 0 and 150° C., optionally conveniently in the presence of bases such as potassium carbonate, sodium carbonate, potassium-tert.-butoxide, sodium ethoxide, potassium hexamethyldisilazane, sodium hydride or lithium diisopropylamide.
b) transition metal-catalysed coupling reaction of a compound of general formula
   A-H (IV),
   wherein A is defined as above, at the aromatic group of general formula 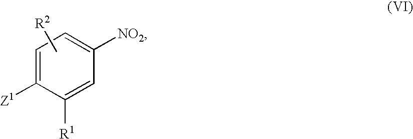
   wherein R¹ and R² are defined as above and Z¹ denotes a chlorine, bromine or iodine atom or a triflate group.

The reaction is expediently carried out in a solvent or mixture of solvents such as benzene, toluene, xylene, tetrahydrofuran, dioxane, diethyl ether, tert.-butyl-methyl-ether, ethyleneglycoldimethylether, diethyleneglycoldimethylether, sulpholane, dimethylformamide, N-methylpyrrolidinone, tetralin, dimethylsulphoxide, methylene chloride, chloroform or tetrachloromethane, for example at temperatures between −30 and 250° C., but preferably between 0 and 150° C., conveniently in the presence of transition metal catalysts such as nickel on activated charcoal, palladium charcoal, tetrakis-(triphenylphosphine)-palladium(0), tris-(dibenzylideneacetone)-dipalladium(0), palladium(II)acetate, palladium(II)chloride, bis-(triphenylphosphine)-palladium(II)-chloride, bis-(tricyclohexylphosphine)-palladium(II)-chloride, bis-(triethylphosphine)-palladium(II)-chloride, bis-(tri-o-tolylphosphine)-palladium(II)-chloride, optionally in the presence of ligands such as triphenylphosphine, tri-o-tolylphosphine, tri-tert.-butylphosphine, 1,3-bis-(diphenylphosphino)-propane, 2,2′-bis-(diphenyl-phosphino)-1,1′-dinaphthyl, 1,1′-bis-(diphenyl phosphino)-ferrocene, Xantphos, and conveniently in the presence of a base such as sodium methoxide, sodium ethoxide, sodium-tert.-butoxide, potassium-tert.-butoxide, sodium-tert.-butyldimethyl-silanoate, potassium hexamethyldisilazane, lithium diisopropylamide, potassium carbonate, rubidium carbonate, caesium carbonate, potassium phosphate, sodium hydride, optionally in the presence of a complexing agent such as 18-crown-6-ether as well as conveniently using an inert gas atmosphere (for example nitrogen or argon) and optionally under pressure.
c) Nucleophilic substitution with a compound of general formula 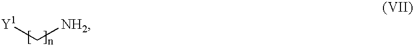
   wherein Y¹ denotes a hydroxyl, amino, hydrazino or thiol function optionally blocked by a corresponding protective group and n is a number between 0 and 4, at the aromatic group of general formula 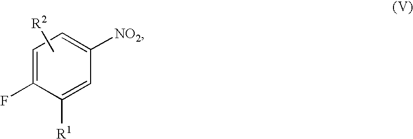
   wherein R¹ and R² are defined as above, and subsequent cyclisation by reaction with a compound of general formula 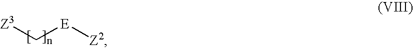
   wherein Z² denotes a carboxylic acid or sulphonic acid protective group such as a methyl or tert.-butyl group and Z³ denotes a nucleofugic leaving group such as chlorine, bromine or iodine atoms or triflate, mesylate or tosylate groups, E denotes the carbonyloxy or sulphonyloxy group and n is a number between 0 and 4, while individual methylene groups may be substituted as described above or replaced by optionally substituted heteroatoms or other groupings.

The initial nucleophilic aromatic substitution of (V) with (VII) is carried out for example as described under (a) 1) i) a).

The subsequent alkylation of the resulting compound with the compound of general formula (VIII) is conveniently carried out in a solvent or mixture of solvents such as benzene, chlorobenzene, toluene, xylene, glycoldimethylether, diethyleneglycoldimethylether, dimethylformamide, N-methylpyrrolidinone, tetralin, dimethylsulphoxide, sulpholane, methylene chloride, chloroform, tetrachloromethane, N-ethyl-diisopropylamine, N-C₁₋₅-alkylmorpholine, N-C₁₋₅-alkylpiperidine, N-C₁₋₅-alkylpyrrolidine, triethylamine, pyridine, for example at temperatures between −30 and 250° C., but preferably between 0 and 150° C., conveniently in the presence of bases such as pyridine, triethylamine, p-dimethylaminopyridine, potassium carbonate, sodium carbonate, potassium tert.-butoxide, sodium methoxide, sodium ethoxide or basic ion exchanger. This may optionally be followed by the unblocking of the nucleophilic group Y¹ and protective group Z² by methods known from the literature or as described in general terms hereinafter.

Cyclisation is then carried out by intramolecular acylation/sulphonylation, conveniently in a solvent or mixture of solvents such as benzene, chlorobenzene, toluene, xylene, glycoldimethylether, diethyleneglycoldimethylether, dimethylformamide, N-methylpyrrolidinone, tetralin, dimethylsulphoxide, sulpholane, methylene chloride, chloroform, tetrachloromethane, N-ethyl-diisopropylamine, N-C₁₋₅-alkylmorpholine, N-C₁₋₅-alkylpiperidine, N-C₁₋₅-alkylpyrrolidine, triethylamine, pyridine, for example at temperatures between −30 and 250° C., but preferably between 0 and 150° C., conveniently in the presence of bases such as pyridine, triethylamine, p-dimethylaminopyridine, potassium carbonate, sodium carbonate, potassium-tert.-butoxide, sodium methoxide, sodium ethoxide or basic ion exchanger.
ii) Transition metal-catalysed coupling reaction of a compound of general formula
   A-H (IV),
   wherein A is defined as above, at the aromatic group of general formula 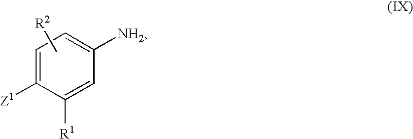
   wherein R¹ and R² are defined as above and Z¹ denotes a chlorine, bromine or iodine atom or a triflate group.

The reaction is expediently carried out in a solvent or mixture of solvents such as benzene, toluene, xylene, tetrahydrofuran, dioxane, diethyl ether, tert.-butyl-methyl-ether, ethyleneglycoldimethylether, diethyleneglycoldimethylether, sulpholane, dimethylformamide, N-methylpyrrolidinone, tetralin, dimethylsulphoxide, methylene chloride, chloroform or tetrachloromethane, for example at temperatures between −30 and 250° C., but preferably between 0 and 200° C., conveniently in the presence of transition metal catalysts such as tetrakis-(triphenylphosphine)-palladium(0), tris-(dibenzylideneacetone)-dipalladium(0), palladium(II)acetate, palladium(II)chloride, bis-(triphenylphosphine)-palladium(II)-chloride, bis-(tricyclohexylphosphine)-palladium(II)-chloride, bis-(triethylphosphine)-palladium(II)-chloride, bis-(tri-o-tolylphosphine)-palladium(II)-chloride, optionally in the presence of ligands such as triphenylphosphine, tri-o-tolylphosphine, tri-tert.-butylphosphine, 1,3-bis-(diphenylphosphino)-propane, 2,2′-bis-(diphenylphosphino)-1,1′-dinaphthyl, 1,1′-bis-(diphenylphosphino)-ferrocene, Xantphos, or for example in the presence of a transition metal catalyst such as copper(I)-iodide, copper(I)-bromide or copper(I)-acetate and conveniently in the presence of a base such as tetramethylguanidine, tetramethylethylenediamine or N,N′-dimethylethylenediamine and conveniently in the presence of a base such as sodium methoxide, sodium ethoxide, sodium-tert.-butoxide, potassium-tert.-butoxide, sodium-tert.-butyldimethyl-silanoate, potassium hexamethyldisilazane, lithium diisopropylamide, potassium carbonate, rubidium carbonate, caesium carbonate, potassium phosphate, sodium hydride, optionally in the presence of a complexing agent such as 18-crown-6-ether as well as conveniently using an inert gas atmosphere (for example nitrogen or argon) and optionally under pressure.
iii) Cyclisation metathesis of a compound of general formula 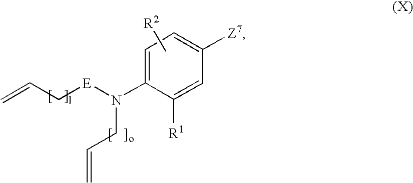
   wherein R¹ and R² are defined as described above, Z⁷ denotes an optionally substituted amino group or the nitro group, E denotes a bond or a methylene, iminocarbonyl or imino group optionally substituted as described above or an oxygen atom, while l and o independently of one another denote identical or different numbers between 1 and 3 which may be obtained for example by a sequence from nucleophilic substitution according to the method described under (a) 1) i) a) and alkylation according to the method described under (a) 1) i) b) with corresponding reagents or other methods known from the literature, optionally followed by reduction, if Z⁷ denotes a nitro group, according to the method described under (a) 1) i).

The cyclisation by a reaction of metathesis is conveniently carried out in a solvent or mixture of solvents such as benzene, chlorobenzene, toluene, xylene, methanol, ethanol, propanol, diethyl ether, tert.-butyl-methyl-ether, tetrahydrofuran, dioxane, glycoldimethylether, diethyleneglycoldimethylether, dimethylformamide, N-methylpyrrolidinone, tetralin, dimethylsulphoxide, sulpholane, methylene chloride, chloroform, tetrachloromethane, pyridine, in the presence of a catalyst such as benzylidene-bis-(tricyclohexylphosphine)-dichloro-ruthenium (1st generation Grubbs catalyst) or benzylidene-[1,3-bis(2,4,6-trimethylphenyl)-2-imidazolidinylidene]-dichloro-(tricyclohexylphosphine)-ruthenium (2nd generation Grubbs catalyst) for example at temperatures between −30 and 250° C., but preferably between 0 and 150° C., conveniently under an inert gas atmosphere, for example argon.

The cyclic systems thus obtained contain a double bond which may be converted into a saturated cyclic compound by hydrogenation with hydrogen, conveniently in a solvent or mixture of solvents such as methanol, ethanol, propanol, ethyl acetate, propylformate, tetrahydrofuran, dioxane, N-methylmorpholine, N-methylpyrrolidine, triethylamine, acetic acid, formic acid, N,N-dimethylformamide or diethyl ether and conveniently in the presence of a catalyst such as Raney nickel, palladium charcoal, platinum, platinum oxide or rhodium on mineral fibres, for example at temperatures between −10 and 250° C., but preferably between 0 and 150° C., optionally with simultaneous reduction of any nitro group found in the molecule, or which may be further derivatised to form the embodiments described above, by a suitable reaction of addition or epoxidation, for example with dimethyldioxirane, m-chloroperbenzoic acid, peracetic acid, hydrogen peroxide in the presence of catalytic amounts of metal oxides such as tungsten oxide or stereoselectively with tert.-butylhydroperoxide in the presence of diethyl (R,R)- or (S,S)-tartrate and titanium(IV)isopropoxide followed by epoxide opening with suitable nucleophils and optionally subsequent derivatisation of the hydroxy groups formed, conveniently in a solvent or mixture of solvents such as water, acetone, ethylformate, ethyl acetate, tert.-butyl-methylether, dichloromethane, chloroform, tetrachloromethane, formic acid or acetic acid at temperatures between −40 and 150° C., preferably between −15 and 90° C.
iv) Hetero-Diels-Alder reaction of a compound of general formula 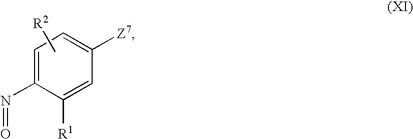
   wherein R¹ and R² are defined as described above, Z⁷ denotes an optionally substituted amino group blocked by a protective group or the nitro group, which may be obtained for example by oxidation of an aniline, for example with potassium peroxodisulphate, by methods known from the literature, with a compound of general formula 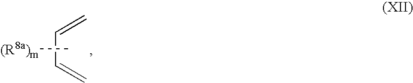
   wherein R^{8a} and m are defined as described above, optionally followed by reduction, if Z⁷ denotes a nitro group, according to the method described under (a) 1) i), or cleaving any protective group which may be present.

Cyclisation by Hetero-Diels-Alder reaction is conveniently carried out in a solvent or mixture of solvents such as methanol, ethanol, propanol, diethyl ether, tert.-butyl-methyl-ether, tetrahydrofuran, dioxane, glycoldimethylether, diethyleneglycoldimethylether, dimethylformamide, N-methylpyrrolidinone, tetralin, dimethylsulphoxide, sulpholane, methylene chloride, chloroform, tetrachloromethane, pyridine, optionally in the presence of a catalyst such as aluminium trichloride, boron trifluoride, zinc chloride, titanium(IV)chloride, lithium perchlorate, ytterbium(III)triflate or chloro-trimethylsilane, for example at temperatures between −30 and 250° C., but preferably between −10 and 150° C. The cyclic systems thus obtained contain a double bond, which may be further derivatised analogously to the the methods described under (a) 1) iii).
2) Preparation of a compound of general formula (II), wherein R³ denotes a C₁₋₃-alkyl group and A and R¹ to R³ are defined as above:
   Reductive amination of a compound of general formula (II), wherein R³ denotes a hydrogen atom and A and R¹ to R³ are as defined above:

The reaction with the corresponding R³-aldehyde (formaldehyde or paraformaldehyde where R³ is methyl, acetaldehyde or paraldehyde where R³ is ethyl, propionaldehyde where R³ is propyl) is conveniently carried out in a solvent or mixture of solvents such as methanol, ethanol, propanol, isopropanol, butanol, tetrahydrofuran, dioxane, diethyl ether, tert.-butyl-methyl-ether, ethyleneglycoldimethylether, diethyleneglycoldimethylether, sulpholane, dimethylformamide, N-methylpyrrolidinone, tetralin, dimethylsulphoxide, methylene chloride, chloroform or tetrachloromethane, for example at temperatures between −30 and 250° C., but preferably between −10 and 150° C., optionally in the presence of a base such as sodium methoxide, sodium ethoxide, sodium-tert.-butoxide, potassium-tert.-butoxide, sodium-tert.-butyldimethyl-silanoate, potassium hexamethyldisilazane, lithium diisopropylamide, potassium carbonate, rubidium carbonate, caesium carbonate, potassium phosphate, sodium hydride, optionally in the presence of a complexing agent such as 18-crown-6-ether, followed by reduction of the resulting imide by hydrogenation with hydrogen, for example under a pressure between 0.5 and 100 bar, but preferably between 1 and 50 bar, conveniently in the presence of a catalyst such as for example Raney nickel, palladium charcoal, platinum oxide, platinum on mineral fibres or rhodium, or with complex hydrides such as lithium aluminium hydride, sodium borohydride, sodium cyanborohydride, diisobutylaluminium hydride, for example at temperatures between −30 and 250° C., but preferably between 0 and 150° C.
(b) In order to prepare a compound of general formula 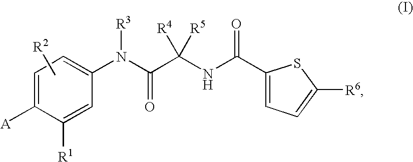
   wherein A and R¹ to R⁶ are as defined above:
1) acylation of a compound of general formula 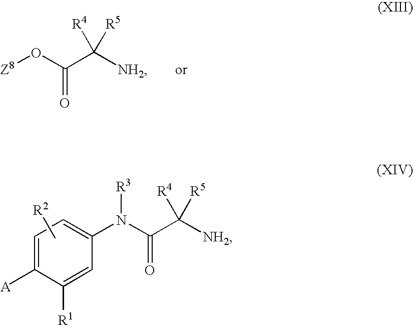
   wherein Z⁸ denotes a protective group for the carboxyl function, which may then be cleaved by methods known from the literature, and A and R¹ to R⁵ are as defined above, while (XIV) may be obtained by the method described under (b) 2), with a carboxylic acid or a reactive carboxylic acid derivative of general formula 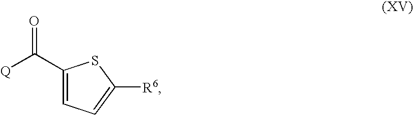
   wherein R⁶ is as defined above and Q denotes a hydroxy or C₁₋₄-alkoxy group, a halogen atom or an acyloxy group.

The acylation is conveniently carried out with a corresponding halide or anhydride in a solvent such as methylene chloride, chloroform, carbon tetrachloride, ether, tetrahydrofuran, dioxane, benzene, toluene, acetonitrile, dimethylformamide, sodium hydroxide solution or sulpholane, optionally in the presence of an inorganic or organic base at temperatures between −20 and 200° C., but preferably at temperatures between −10 and 160° C.

The acylation may however also be carried out with the free acid, optionally in the presence of an acid-activating agent or a dehydrating agent, for example in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole, O-(benzotriazol-1-yl)-N,N,N′,N′-tetramethyl-uroniumtetrafluoroborate/N-methylmorpholine, O-(benzotriazol-1-yl)-N,N,N′,N′-tetramethyl-uronium tetrafluoroborate/N-ethyldiisopropylamine, O-pentafluorophenyl-N,N,N′,N′-tetramethyluronium-hexafluorophosphate/triethylamine, N,N′-thionyldiimidazole or triphenylphosphine/carbon tetrachloride, at temperatures between −20 and 200° C., but preferably at temperatures between −10 and 160° C.
2) Acylation of a compound of general formula 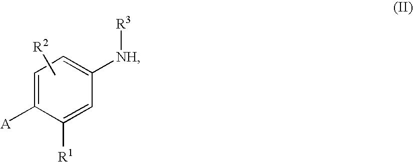
   wherein A and R¹ to R³ are as defined above,
      with a carboxylic acid or a reactive carboxylic acid derivative of general formula 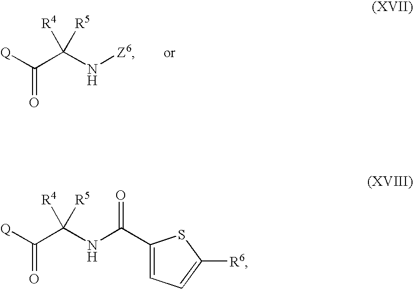
      wherein R⁴ to R⁶ are as defined above, Q denotes a hydroxy or C₁₋₄-alkoxy group, a halogen atom or an acyloxy group and Z⁶ denotes a protective group, which may subsequently be cleaved by methods known from the literature, while (XVIII) may be obtained according to the method described under (b) 1).

The acylation may be carried out analogously to the method described under (b) 1).

The acylation may however also conveniently be carried out in a solvent or mixture of solvents such as dichloromethane, trichloromethane, benzene, chlorobenzene, toluene, xylene, hexamethyldisiloxane, acetonitrile, N-ethyl-diisopropylamine, N-C₁₋₅-alkylmorpholine, N-C₁₋₅-alkylpiperidine, N-C₁₋₅-alkylpyrrolidine, triethylamine, pyridine, in the presence of 4-trifluoromethyl-benzoic acid-anhydride, silver triflate and titanium (IV)chloride, conveniently in the presence of a dehydrating agent such as molecular sieve, sodium sulphate, magnesium sulphate, or in the presence of 4-trifluoromethyl-benzoic acid-anhydride and ylterbium(III)triflate, while water may also be added to the solvent mixture, for example at temperatures between −30 and 250° C., but preferably between 0 and 150° C., (I. Shiina, M. Miyashita, M. Nagai, T. Mukaiyama; Heterocycles 1995, 40 (1), 141-148.).

Other methods of amide coupling are described for example in P. D. Bailey, I. D. Collier, K. M. Morgan in “Comprehensive Functional Group Interconversions”, Vol. 5, page 257ff., Pergamon 1995.

In the reactions described above any reactive groups present such as hydroxy, carboxy, amino, alkylamino or imino groups may be protected during the reaction by conventional protective groups which are cleaved again after the reaction.

For example a suitable protective group for a hydroxy group is the methoxy, benzyloxy, trimethylsilyl, acetyl, benzoyl, tert.-butyl, trityl, benzyl or tetrahydro-pyranyl group,
a suitable protective group for a carboxyl group is the trimethylsilyl, methyl, ethyl, tert.-butyl, benzyl or tetrahydropyranyl group and
a suitable protective group for an amino, alkylamino or imino group is the acetyl, trifluoroacetyl, benzoyl, ethoxycarbonyl, tert.-butoxycarbonyl, benzyloxycarbonyl, benzyl, methoxybenzyl or 2,4-dimethoxybenzyl group and additionally a suitable protective group for the amino group is the phthalyl group.

Other protective groups and their cleaving are described in T. W. Greene, P. G. M. Wuts, “Protective Groups in Organic Synthesis”, Wiley, 1991 and 1999.

Any protective group used is optionally subsequently cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by means of ether splitting, e.g. in the presence of iodotrimethylsilane, at temperatures between 0 and 100° C., preferably at temperatures between 10 and 50° C.

A benzyl, methoxybenzyl or benzyloxycarbonyl group, however, is cleaved by hydrogenolysis, for example, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and under a hydrogen pressure of 1 to 7 bar, but preferably 1 to 5 bar.

A methoxybenzyl group may also be cleaved in the presence of an oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisol.

A tert.-butyl or tert.-butyloxycarbonyl group is preferably cleaved by treatment with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treatment with a catalytic amount of tetrakis-(triphenylphosphine)-palladium(0), preferably in a solvent such as tetrahydrofuran and preferably in the presence of an excess of a base such as morpholine or 1,3-dimedone at temperatures between 0 and 100° C., preferably at ambient temperature and under inert gas, or by treatment with a catalytic amount of tris-(triphenylphosphine)-rhodium(I)chloride in a solvent such as aqueous ethanol and optionally in the presence of a base such as 1,4-diazabicyclo[2.2.2]octane at temperatures between 20 and 70° C.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned above, the compounds of general formula I and the tautomers, enantiomers, diastereomers and physiologically acceptable salts thereof have valuable pharmacological properties, particularly an antithrombotic activity which is preferably based on an effect on thrombin or factor Xa, for example on a thrombin-inhibiting or factor Xa-inhibiting activity, on a prolonging effect on the aPTT time and/or on an inhibitory effect on related serine proteases such as e.g. urokinase, factor VIIa, factor IXa, factor XIa and factor XIIa.

The compounds listed in the Experimental Section were investigated for their effect on the inhibition of factor Xa as follows:

Method:

Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mMol) and sodium chloride (150 mMol), pH 8.0 plus 1 mg/ml Human Albumin Fraction V, protease-free
Factor Xa (Calbiochem), spec. activity: 217 IU/mg, final concentration: 7 IU/ml for each reaction mixture
Substrate S 2765 (Chromogenix), final concentration: 0.3 mM/I (1 KM) for each reaction mixture
Test substance: final concentration 100, 30, 10, 3, 1, 0.3, 0.1, 0.03, 0.01, 0.003, 0.001 μMol/l
   Procedure:

10 μl of a 23.5-times concentrated starting solution of the test substance or solvent (control), 175 μl of TRIS/HSA buffer and 25 μl of a 65.8 U/L Factor Xa working solution are incubated for 10 minutes at 37° C. After the addition of 25 μl of S 2765 working solution (2.82 mMol/l) the sample is measured in a photometer (SpectraMax 250) at 405 nm for 600 seconds at 37° C.

Evaluation:

1. Determining the maximum increase (deltaOD/minutes) over 21 measuring points.
2. Determining the % inhibition based on the solvent control.
3. Plotting a dosage/activity curve (% inhibition vs substance concentration).
4. Determining the IC₅₀ by interpolating the X-value (substance concentration) of the dosage/activity curve at Y=50% inhibition.

All the compounds tested had an IC₅₀ value of less than 100 μmol/L.

The compounds prepared according to the invention are generally well tolerated.

In view of their pharmacological properties the new compounds and the physiologically acceptable salts thereof are suitable for the prevention and treatment of venous and arterial thrombotic diseases, such as for example the prevention and treatment of deep leg vein thrombosis, for preventing reocclusions after bypass operations or angioplasty (PT(C)A), and occlusion in peripheral arterial diseases, and for preventing and treating pulmonary embolism, disseminated intravascular coagulation and severe sepsis, for preventing and treating DVT in patients with exacerbated COPD, for treating ulcerative colitis, for preventing and treating coronary thrombosis, for preventing stroke and the occlusion of shunts.

In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic incidents in patients with all forms of coronary heart disease, for preventing metastasis and the growth of tumours and inflammatory processes, e.g. in the treatment of pulmonary fibrosis, for preventing and treating rheumatoid arthritis, for preventing and treating fibrin-dependent tissue adhesions and/or the formation of scar tissue and for promoting wound healing processes.

In view of their pharmacological properties the new compounds and the physiologically acceptable salts thereof are also suitable for the treatment of Alzheimer's and Parkinson's disease. One explanation for this arises for example from the following findings, from which it can be concluded that thrombin inhibitors or factor Xa inhibitors, by inhibiting thrombin formation or thrombin activity, may be valuable drugs for treating Alzheimer's and Parkinson's disease. Clinical and experimental studies indicate that neurotoxic mechanisms, for example the inflammation which is associated with the activation of proteases of the clotting cascade, are involved in the dying of neurones following brain injury. Various studies point to the involvement of thrombin in neurodegenerative processes, for example following a stroke, repeated bypass operations or traumatic brain injury. An increased thrombin activity has been demonstrated some days after peripheral nerve damage, for example. It has also been shown that thrombin causes a neurite retraction, as well as glia proliferation, and apoptosis in primary cultures of neurones and neuroblastoma cells (for a summary see: Neurobiol. Aging 2004, 25(6), 783-793). Moreover, various in vitro studies on the brains of patients with Alzheimer's disease indicated that thrombin plays a role in the pathogenesis of this disease (Neurosci. Lett. 1992, 146,152-54). A concentration of immune-reactive thrombin has been detected in neurite plaques in the brains of Alzheimer's patients. It has been dmonstrated in vitro that thrombin also plays a part in the regulation and stimulation of the production of the “Amyloid Precursor Protein” (APP) as well as in the cleaving of the APP into fragments which can be detected in the brains of Alzheimer's patients. Moreover, it has been demonstrated that the thrombin-induced microglial activation leads in vivo to the degeneration of nigral dopaminergic neurones. These findings lead one to conclude that microglial activation, triggered by endogenous substance(s) such as thrombin, for example, are involved in the neuropathological process of the cell death of dopaminergic neurones of the kind which occurs in patients with Parkinson's disease (J. Neurosci. 2003, 23, 5877-86).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day.

For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The new compounds and the physiologically acceptable salts thereof may be used therapeutically in conjunction with acetylsalicylic acid, with inhibitors of platelet aggregation such as fibrinogen receptor antagonists (e.g. abciximab, eptifibatide, tirofiban, roxifiban), with physiological activators and inhibitors of the clotting system and the recombinant analogues thereof (e.g. Protein C, TFPI, antithrombin), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The Examples that follow are intended to illustrate the invention, without restricting its scope:

### EXPERIMENTAL SECTION

As a rule, melting points, IR, UV, ¹H-NMR and/or mass spectra have been obtained for the compounds prepared. Unless otherwise stated, R_{f} values were determined using ready-made silica gel 60 F₂₅₄ TLC plates (E. Merck, Darmstadt, Item no. 1.05714) without chamber saturation. The R_{f} values given under the heading Alox were determined using ready-made aluminium oxide 60 F₂₅₄ TLC plates (E. Merck, Darmstadt, Item no. 1.05713) without chamber saturation. The R_{f} values given under the heading Reversed-phase-8 were determined using ready-made RP-8 F₂₅₄ₛ TLC plates (E. Merck, Darmstadt, Item no. 1.15684) without chamber saturation. The ratios given for the eluants refer to units by volume of the solvents in question. For chromatographic purification silica gel made by Messrs Millipore (MATREX™, 35-70 my) was used. Unless more detailed information is provided as to the configuration, it is not clear whether the products are pure stereoisomers or mixtures of enantiomers and diastereomers.

The following abbreviations are used in the descriptions of the experiments:

| | |
|---|---|
| Boc | tert.-butoxycarbonyl |
| DIPEA | N-ethyl-diisopropylamine |
| DMSO | dimethylsulphoxide |
| DMF | N,N-dimethylformamide |
| sat. | saturated |
| h | hour(s) |
| HATU | O-(7-azabenzotriazol-1-yl)-N,N,N′,N′-tetramethyluronium- |
| | hexafluorophosphate |
| i. vac. | in vacuo |
| conc. | concentrated |
| min | minute(s) |
| NMM | N-methyl-morpholine |
| NMP | N-methyl-pyrrolidin-2-one |
| o | ortho |
| PfTU | O-pentafluorophenyl-N,N,N′,N′-tetramethyluronium- |
| | hexafluorophosphate |
| PPA | propanephosphonic acid cycloanhydride |
| quant. | quantitative |
| R_{f} | retention factor |
| Rₜ | retention time |
| rac. | racemic |
| TBTU | O-(benzotriazol-1-yl)-N,N,N′,N′-tetramethyluronium |
| | tetrafluoroborate |
| TEA | triethylamine |
| TFA | trifluoroacetic acid |
| THF | tetrahydrofuran |
| tert. | tertiary |
| Σ | yield over all the steps carried out analogously as described |

The HPLC/MS data for Examples 24 to 26 were obtained under the following conditions:
(a) HP1100 HPLC+diode array detector with Waters ZQ2000 mass spectrometer and Gilson 215 Autosampler

The mobile phase used was:
A: water with 0.1% TFA

B: acetonitrile with 0.1% TFA

| time in min | % A | % B | flow rate in ml/min |
|---|---|---|---|
| 0.0 | 95 | 5 | 1.00 |
| 0.4 | 95 | 5 | 1.00 |
| 4.0 | 2 | 98 | 1.00 |
| 4.35 | 2 | 98 | 1.00 |
| 4.5 | 95 | 5 | 1.00 |

The stationary phase used was a Waters column X-Terra™ MS C₁₈ 3.5 μm, 4.6 mm×50 mm (column temperature: constant at 40° C.)

The diode array detection took place in a wavelength range from 210-500 nm Range of mass-spectrometric detection: m/z 120 to m/z 1000

Where specified, the HPLC data of the other Examples were obtained under the following conditions:
(b) Waters ZMD, Alliance 2695 HPLC, Waters 2700 Autosampler, Waters 996 diode array detector

The mobile phase used was:
A: water with 0.13% TFA

B: acetonitrile with 0.10% TFA

| time in min | % A | % B | flow rate in ml/min |
|---|---|---|---|
| 0.0 | 95 | 5 | 1.00 |
| 0.7 | 95 | 5 | 1.00 |
| 5.2 | 2 | 98 | 1.00 |
| 5.7 | 2 | 98 | 1.00 |
| 6.0 | 95 | 5 | 1.00 |
| 6.5 | 95 | 5 | 1.00 |

The stationary phase used was a Varian column, Microsorb 100 C₁₈ 3 μm, 4.6 mm×50 mm, batch no. 2231108 (column temperature: constant at 25° C.).

### Example 1

### 5-chloro-thiophene-2-carboxylic acid-N-{(1R)-2-benzyloxy-1-[3-chloro-4-(4-methyl-[1,4]diazepan-1-yl)-phenylcarbamoyl)-ethyl}-amide

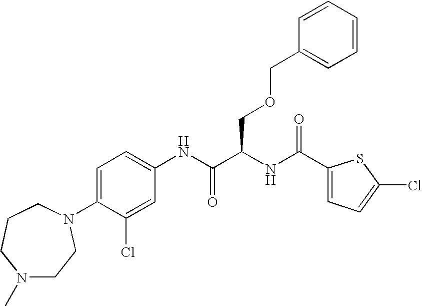

### (a) 3-chloro-4-(4-methyl-[1.4]diazepan-1-yl)-1-nitro-benzene

2.00 g (11.4 mmol) 3-chloro-4-fluoro-1-nitro-benzene are combined with 1.42 ml (11.4 mmol) 1-methyl-[1,4]diazepan in 20 ml DMSO with stirring at ambient temperature, heated to 90° C. for 2 hours and stirred for 16 hours at ambient temperature. After evaporation i. vac. the residue is combined with water, the precipitate formed is suction filtered, dissolved in ethyl acetate, dried over sodium sulphate and applied to silica gel i. vac. The residue is purified by chromatography on silica gel (eluant gradient: dichloromethane/methanol=95:5->70:30).

Yield: 2.77 g (90%)

R_{f} value: 0.17 (silica gel; dichloromethane/methanol=95:5)

C₁₂H₁₆ClN₃O₂ (269.73)

Mass spectrum: (M+H)⁺=270/272 (chlorine isotope)

### (b) 3-chloro-4-(4-methyl-[1.4]diazepan-1-yl)-aniline

1.00 g (3.71 mmol) 3-chloro-4-(4-methyl-[1,4]diazepan-1-yl)-1-nitro-benzene are combined with 370 mg Raney nickel in 75 ml of ethyl acetate and hydrogenated in a Parr apparatus at ambient temperature for 1.5 h at 50 psi hydrogen pressure. Then the Raney nickel is filtered off and the filtrate is evaporated down i. vac. The residue is purified by chromatography on silica gel (eluant gradient: dichloromethane/methanol=90:10->0:100).

Yield: 630 mg (71%)

R_{f} value: 0.14 (silica gel; dichloromethane/methanol=9:1)

C₁₂H₁₈ClN₃ (239.74)

Mass spectrum: (M+H)⁺=240/242 (chlorine isotope)

### (c) (2R)-3-benzyloxy-2-Boc-amino-N-[3-chloro-4-(4-methyl-[1,4]diazepan-1-yl)-phenyl]-propionic acid-amide

777 mg (19.1 mmol) O-benzyl-N-Boc-D-serine are combined with 400 μl (3.6 mmol) NMM and 930 mg (2.9 mmol) TBTU in 3 ml DMF and then stirred for 15 min under a nitrogen atmosphere at ambient temperature. Then 630 mg (2.6 mmol) 3-chloro-4-(4-methyl-[1,4]diazepan-1-yl)-aniline are added and the mixture is stirred for a further 2 hours at ambient temperature. Then the reaction mixture is poured into water, extracted with ethyl acetate, the combined organic phases are dried over sodium sulphate and evaporated down completely i. vac.

Yield: 1.58 g (quant.)

R_{f} value: 0.90 (silica gel; dichloromethane/methanol=4:1)

C₂₇H₃₇ClN₄O₄ (517.06)

Mass spectrum: (M+H)⁺=517/519 (chlorine isotope)

### (d) (2R)-2-amino-3-benzyloxy-N-[3-chloro-4-(4-methyl-[1,4]diazepan-1-yl)-phenyl]-propionic acid-amide

1.58 g (3.06 mmol) (2R)-3-benzyloxy-2-Boc-amino-N-[(3-chloro-4-(4-methyl-[1,4]diazepan-1-yl)-phenyl]-propionic acid-amide are combined with 19 ml (114 mmol) 6-molar hydrochloric acid in 6 ml dioxane and then stirred for 30 min at ambient temperature. Then ethyl acetate and water is added and the mixture is extracted with ethyl acetate. The aqueous phase is made alkaline with ammonia solution and extracted with ethyl acetate. The combined organic phases are dried over sodium sulphate and evaporated down completely.

Yield: 930 mg (73%)

R_{f} value: 0.34 (silica gel; dichloromethane/methanol=4:1)

C₂₂H₂₉ClN₄O₂ (416.94)

Mass spectrum: (M+H)⁺=417/419 (chlorine isotope)

### (e) N-[(1R)-2-benzyloxy-1-(3-chloro-4-(4-methyl-[1.4]diazepan-1-yl)-phenylcarbamoyl)-ethyl]-5-chloro-thiophene-2-carboxylic acid-amide

Prepared analogously to Example 1c from 5-chloro-thiophene-2-carboxylic acid and (2R)-2-amino-3-benzyloxy-N-[3-chloro-4-(4-methyl-[1,4]diazepan-1-yl)-phenyl]-propionic acid-amide with TBTU and NMM in DMF.

Yield: 65%

Rₜ value: 2.93 min

C₂₇H₃₀Cl₂N₄O₃S*CF₃COOH (675.55/561.53)

Mass spectrum: (M+H)⁺=560/562/564 (chlorine isotope)

The following compounds were prepared analogously:

| | Structural formula | | | |
|---|---|---|---|---|
| No. | Name | Yield | Mass peak(s) | R_{f} value or Rₜ |
| 4 | 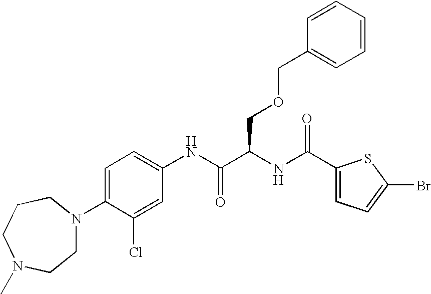 | Σ: 16% | (M + H)⁺ = 606/608/610 (bromine and chlorine isotopes) | 2.90 min |
| | 5-bromo-thiophene-2-carboxylic acid-N-{(1R)-2-benzyloxy-1-[3-chloro-4-(4-meth- | | | |
| | yl-[1,4]diazepan-1-yl)-phenylcarbamoyl]-ethyl}-amide | | | |
| 5 | 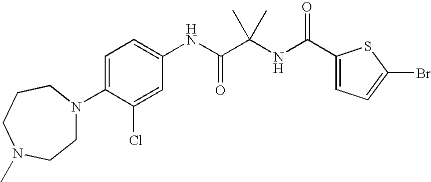 | Σ: 2.5% | (M + H)⁺ = 514/516/518 (bromine and chlorine isotopes) | 4.20 min |
| | 5-bromo-thiophene-2-carboxylic acid-N-{1-[3-chloro-4-(4-methyl-[1,4]diaze- | | | |
| | pan-1-yl)-phenylcarbamoyl]-1-methyl-ethyl}-amide | | | |
| 6 | 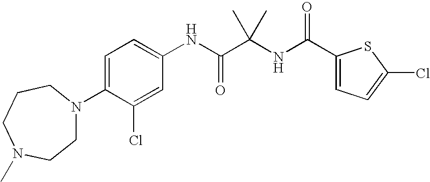 | Σ: 2.4% | (M + H)⁺ = 470/472/474 (chlorine isotope) | 4.09 min |
| | 5-chloro-thiophene-2-carboxylic acid-N-{1-[3-chloro-4-(4-methyl-[1,4]diaze- | | | |
| | pan-1-yl)-phenylcarbamoyl]-1-methyl-ethyl}-amide | | | |
| 11 | 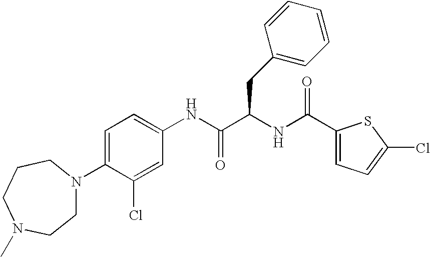 | Σ: 4.1% | (M+ H)⁺ = 531/533/535 (chlorine isotope) | 0.14 (silica gel; dichloromethane/ methanol = 9:1) |
| | 5-chloro-thiophene-2-carboxylic acid-N-{(1R)-1-[3-chloro-4-(4-methyl-[1,4]diaze- pan-1-yl)-phenylcarbamoyl]-2-phenyl-ethyl}-amide | | | |
| 12 | 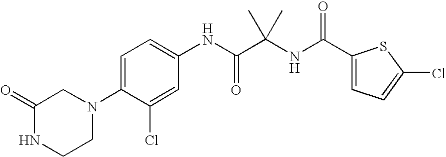 | Σ: 2.9% | (M + H)⁺ = 455/457/459 (chlorine isotope) | 0.40 (silica gel; dichloromethane/ methanol = 9:1) |
| | 5-chloro-thiophene-2-carboxylic acid-N-{1-[3-chloro-4-(3-oxo-piperazin-1-yl)-phenyl- | | | |
| | carbamoyl]-1-methyl-ethyl}-amide | | | |
| 13 | 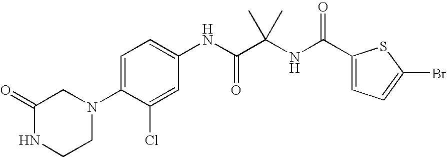 | Σ: 2.8% | (M + H)⁺ = 499/501/503 (bromine and chlorine isotopes) | 0.45 (silica gel; dichloromethane/ methanol = 9:1) |
| | 5-bromo-thiophene-2-carboxylic acid-N-{1-[3-chloro-4-(3-oxo-piperazin-1-yl)-phenyl- | | | |
| | carbamoyl]-1-methyl-ethyl}-amide | | | |
| 15 | 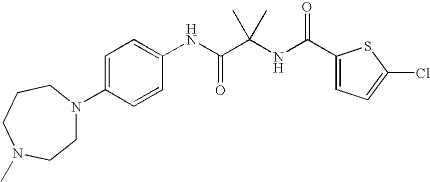 | Σ: 0.8% | (M + H)⁺ = 435/437 (chlorine isotope) | 4.01 min |
| | 5-chloro-thiophene-2-carboxylic acid-N-{1-[4-(4-methyl-[1,4]diazepan-1-yl)-phenyl- | | | |
| | carbamoyl]-1-methyl-ethyl}-amide | | | |
| 16 | 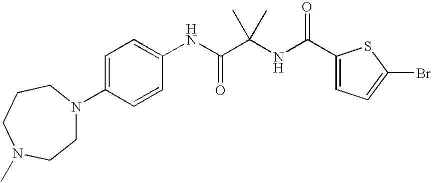 | Σ: 0.9% | (M + H)⁺ = 479/481 (bromine isotope) | 4.00 min |

5-bromo-thiophene-2-carboxylic acid-N-{1-[4-(4-methyl-[1,4]diazepan-1-yl)-phenyl-
carbamoyl]-1-methyl-ethyl}-amide

The following compounds may be prepared analogously:
(1) 5-bromo-thiophene-2-carboxylic acid-N-{(1R)-1-[3-chloro-4-(pyrazolidin-1-yl)-phenylcarbamoyl]-2-methoxy-ethyl)amide,
(2) 5-bromo-thiophene-2-carboxylic acid-N-(1R)-1-[3-chloro-4-(tetrahydropyridazin-1-yl)-phenylcarbamoyl]-2-methoxy-ethyl}-amide,
(3) 5-bromo-thiophene-2-carboxylic acid-N-{1-[3-chloro-4-(2-oxo-piperazin-4-yl)-phenylcarbamoyl]-ethyl}-amide,
(4) 5-bromo-thiophene-2-carboxylic acid-N-{1-[3-chloro-4-(tetrahydropyridazin-3-on-1-yl)-phenylcarbamoyl]-ethyl}-amide,
(5) 5-chloro-thiophene-2-carboxylic acid-N-{(1R)-1-[3-methyl-4-(4-methyl-[1,4]diazepan-1-yl)-phenylcarbamoyl]-3-methylsulphanyl-propyl)amide,
(6) 5-chloro-thiophene-2-carboxylic acid-N-{1-[3-methyl-4-(4-methyl-[1,4]diazepan-1-yl)-phenylcarbamoyl]-1-phenyl-methyl}-amide,
(7) 5-chloro-thiophene-2-carboxylic acid-N-{1-[3-methyl-4-(4-methyl-[1,4]diazepan-1-yl)-phenylcarbamoyl]-1-[pyridin-3-yl]-methyl}-amide,
(8) 5-chloro-thiophene-2-carboxylic acid-N-{1-[3-methyl-4-(4-methyl-[1,4]diazepan-1-yl)-phenylcarbamoyl]-2-[imidazol-4-yl]-ethyl}-amide,
(9) 5-chloro-thiophene-2-carboxylic acid-N-{1-[3-methyl-4-(4-methyl-[1,4]diazepan-1-yl)-phenylcarbamoyl]-3-(dimethylaminocarbonyl)-propyl}-amide,

### Example 2

### 5-chloro-thiophene-2-carboxylic acid-N-{1-[3-bromo-4-(4-methyl-piperazin-1-yl)-phenylcarbamoyl]-ethyl)-amide

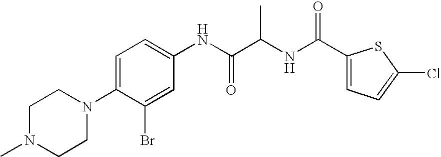

Prepared analogously to Example 1c from 2-[(5-chloro-thiophene-2-carbonyl)-amino]-propionic acid and 3-bromo-4-(4-methyl-piperazin-1-yl)-aniline with TBTU and TEA in DMF.

Yield: 44%

R_{f} value: 0.31 (silica gel; dichloromethane/methanol=9:1)

C₁₉H₂₂BrClN₄O₂S (485.83)

Mass spectrum: (M+H)⁺=485/487/489 (bromine and chlorine isotopes)

### Example 3

### 5-chloro-thiophene-2-carboxylic acid-N-(1R)-1-[3-chloro-4-(4-methyl-[1,4]diazepan-1-yl)-phenylcarbamoyl]-2-hydroxy-ethyl}-amide

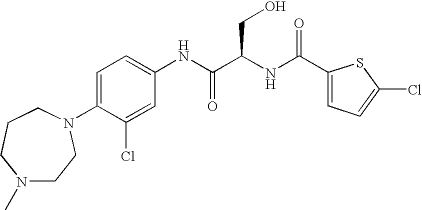

200 mg (0.30 mmol) 5-chloro-thiophene-2-carboxylic acid-N-{(1R)-2-benzyloxy-1-[3-chloro-4-(4-methyl-[1,4]diazepan-1-yl)-phenylcarbamoyl]-ethyl}-amide-trifluoroacetate in 1.65 ml TFA are combined with 445 mg (3.0 mmol) pentamethylbenzene and heated to 50° C. for 9.5 hours with stirring and stirred for 60 hours at ambient temperature. The mixture is concentrated by evaporation, taken up in acetonitrile, acidified with TFA and purified by HPLC.

Yield: 62 mg (36%)

Rₜ value: 2.37 min

C₂₀H₂₄Cl₂N₄O₃S*CF₃COOH (585.42/471.40)

Mass spectrum: (M+H)⁺=472/474/476 (chlorine isotope)

### Example 7

### 5-bromo-thiophene-2-carboxylic acid-N-1-methyl-1-[3-trifluoromethyl-4-(4-methyl-[1,4]diazepan-1-yl)-phenylcarbamoyl]-ethyl)amide

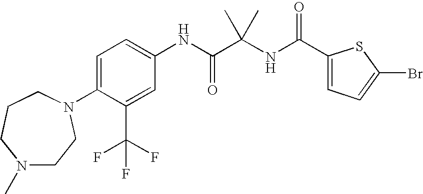

### (a) tert.butyl 2-[(5-bromo-thiophene-2-carbonyl)-amino]-2-methyl-propionate

Prepared analogously to Example 1c from 5-bromo-thiophene-2-carboxylic acid and tert.butyl 2-amino-2-methyl-propionate with TBTU and TEA in DMF.

Yield: 80%

R_{f} value: 0.85 (silica gel; petroleum ether/ethyl acetate=1:1)

C₁₃H₁₈BrNO₃S (348.26)

Mass spectrum: (M+H)⁺=348/350 (bromine isotope)

### (b) 2-[(5-bromo-thiophene-2-carbonyl)-amino]-2-methyl-propionic acid

31.0 g (89.0 mmol) tert.butyl 2-[(5-bromo-thiophene-2-carbonyl)-amino]-2-methyl-propionate are stirred in 125 ml dichloromethane with 45 ml trifluoroacetic acid for 18 h at ambient temperature, then refluxed for 1 h, a further 10 ml trifluoroacetic acid is added and the mixture is refluxed for a further 2 h. Then it is evaporated down i. vac., the residue is taken up twice in toluene and evaporated down completely.

Yield: 24.7 g (95%)

R_{f} value: 0.30 (silica gel; petroleum ether/ethyl acetate=1:1+1% acetic acid)

C₉H₁₀BrNO₃S (292.15)

Mass spectrum: (M+H)⁺=292/294 (bromine isotope)

### (c) 3-trifluoromethyl-4-(4-methyl-[1.4]diazepan-1-yl)-1-nitro-benzene

Prepared analogously to Example 1a from 4-fluoro-3-trifluoro-1-nitro-benzene and 1-methyl-[1,4]diazepan with potassium carbonate in DMF.

Yield: 45%

R_{f} value: 0.20 (silica gel; dichloromethane/methanol=95:5)

C₁₃H₁₆F₃N₃O₂ (303.28)

Mass spectrum: (M+H)⁺=304

### (d) 3-trifluoromethyl-4-(4-methyl-[1,4]diazepan-1-yl)-aniline

520 mg (1.72 mmol) 3-trifluoromethyl-4-(4-methyl-[1,4]diazepan-1-yl)-1-nitro-benzene are combined with 60 mg 20% palladium charcoal in 10 ml of methanol and hydrogenated in a Parr apparatus at ambient temperature for 3.5 h at 50 psi hydrogen pressure. Then the palladium charcoal is filtered off and the filtrate is evaporated down i. vac. The residue is further reacted without any more purification.

Yield: 402 mg (86%)

Rₜ value: 1.71 min

C₁₃H₁₈F₃N₃ (273.30)

Mass spectrum: (M+H)⁺=274

### (e) 5-bromo-thiophene-2-carboxylic acid-N-[1-methyl]-[3-trifluoromethyl-4-(4-methyl-[1,4]diazepan-1-yl)-phenyl-carbamoyl]-ethyl]-amide

Prepared analogously to Example 1c from 2-[(5-bromo-thiophene-2-carbonyl)-amino]-2-methyl-propionic acid and 3-trifluoromethyl-4-(4-methyl-[1,4]diazepan-1-yl)-aniline with TBTU and NMM in DMF.

Yield: 25%

Rₜ value: 2.65 min

C₂₂H₂₆BrF₃N₄O₂S*CF₃COOH (547.39/661.46)

Mass spectrum: (M+H)⁺=547/549 (bromine isotope)

The following compound was prepared analogously:

| | Structural formula | | | |
|---|---|---|---|---|
| No. | Name | Yield | Mass peak(s) | R_{f} value or Rₜ |
| 9 | 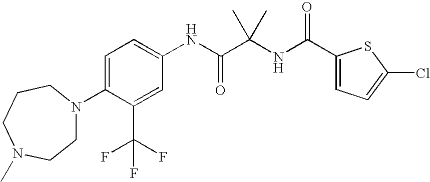 | Σ: 13% | (M + H)⁺ = 503/505 (chlorine isotope) | 0.58 (silica gel; petroleum ether/ ethyl acetate =7:3) |

5-chloro-thiophene-2-carboxylic acid-N-{1-methyl-1-[3-trifluoromethyl-4-(4-meth-
yl-[1,4]diazepan-1-yl)-phenylcarbamoyl]-ethyl}-amide

The following compounds may be prepared analogously:
(1) 5-bromo-thiophene-2-carboxylic acid-N-{1-[3-chloro-4-(pyrazolidin-1-yl)-phenylcarbamoyl]-1-methyl-ethyl}-amide,
(2) 5-chloro-thiophene-2-carboxylic acid-N-{1-[3-methyl-4-(pyrazolidin-1-yl)-phenylcarbamoyl]-1-methyl-ethyl}-amide,
(3) 5-bromo-thiophene-2-carboxylic acid-N-{1-[3-chloro-4-(tetrahydropyridazin-1-yl)-phenylcarbamoyl]-1-methyl-ethyl}-amide,
(4) 5-chloro-thiophene-2-carboxylic acid-N-{1-[3-methyl-4-(tetrahydropyridazin-1-yl)-phenylcarbamoyl]-1-methyl-ethyl}-amide,
(5) 5-bromo-thiophene-2-carboxylic acid-N-{1-[3-bromo-4-(4-methyl-[1,4]diazepan-1-yl)-phenylcarbamoyl]-1-methyl-ethyl}-amide,
(6) 5-bromo-thiophene-2-carboxylic acid-N-{1-[3-trifluoromethoxy-4-(4-methyl-[1,4]diazepan-1-yl)-phenylcarbamoyl]-1-methyl-ethyl}-amide,
(7) 5-bromo-thiophene-2-carboxylic acid-N-{1-methyl-[3-methyl-4-(2,5-dimethyl-pyrrolidin-1-yl)-phenylcarbamoyl]-ethyl}-amide,
(8) 5-bromo-thiophene-2-carboxylic acid-N-{1-[3-methyl-4-(2,6-dimethyl-piperidin-1-yl)-phenylcarbamoyl]-cyclopentyl}-amide,
(9) 5-bromo-thiophene-2-carboxylic acid-N-{4-[3-methyl-4-(2,6-dimethyl-piperidin-1-yl)-phenylcarbamoyl]-tetrahydropyran-4-yl}-amide,
(10) 5-bromo-thiophene-2-carboxylic acid-N-{1-methyl-4-[3-methyl-4-(2,6-dimethyl-morpholin-4-yl)-phenylcarbamoyl]-piperazin-4-yl}-amide,
(11) 5-bromo-thiophene-2-carboxylic acid-N{-3-[3-chloro-4-(2,2-dimethyl-pyrrolidin-1-yl)-phenylcarbamoyl]-tetrahydrofuran-3-yl}-amide,

### Example 8

### 5-bromo-thiophene-2-carboxylic acid-N-{1-[3-chloro-4-(morpholin-4-yl)-phenylcarbamoyl]-1-methyl-ethyl}-amide

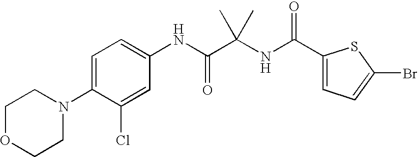

206 mg (0.71 mmol) 2-[(5-bromo-thiophene-2-carbonyl)-amino]-2-methyl-propionic acid together with 295 mg (0.78 mmol) HATU in 3.0 ml DMF are combined with 155 μl (1.41 mmol) NMM with stirring at ambient temperature and stirred for 45 min. Then 150 mg 3-chloro-4-(morpholin-4-yl)-aniline are added and the mixture is heated to 85° C. for 3 h. After stirring at ambient temperature for 16 h the mixture is poured onto ice with sat. sodium hydrogen carbonate solution and extracted with ethyl acetate. The combined organic phases are washed with 0.5-molar potassium hydrogen sulphate solution and sat. sodium chloride solution, dried over magnesium sulphate and evaporated down completely i. vac. The residue is triturated in diethyl ether, filtered off and dried at ambient temperature.

Yield: 235 mg (68%)

R_{f} value: 0.20 (silica gel; dichloromethane/methanol=9:1)

C₁₉H₂₁BrClN₃O₃S (486.81)

Mass spectrum: (M+H)⁺=486/488/490 (bromine and chlorine isotopes)

The following compounds were prepared analogously:

| | Structural formula | | | |
|---|---|---|---|---|
| No. | Name | Yield | Mass peak(s) | R_{f} value or Rₜ |
| 14 | 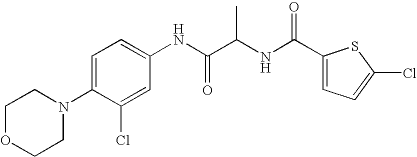 | 71% | (M + H)⁺ = 428/430/432 (chlorine isotope) | 0.53 (silica gel; dichloromethane/ methanol = 9:1) |
| | 5-chloro-thiophene-2-carboxylic acid-N-{1-[3-chloro-4-(morpholin-4-yl)-phenyl- | | | |
| | carbamoyl]-ethyl}-amide | | | |
| 26 | 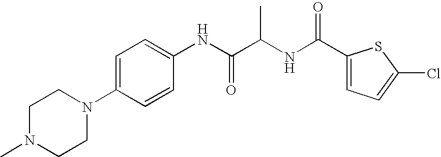 | quant. | (M + H)⁺ = 407/409 (chlorine isotope) | 3.15 min |

5-chloro-thiophene-2-carboxylic acid-N-{1-[3-chloro-4-(morpholin-4-yl)-phenyl-
carbamoyl]-ethyl}-amide

The following compounds may be prepared analogously:
(1) 5-chloro-thiophene-2-carboxylic acid-N-{1-[3-methyl-4-(morpholin-4-yl)-phenylcarbamoyl]-1-methyl-ethyl}-amide,
(2) 5-bromo-thiophene-2-carboxylic acid-N-{(1R)-1-[3-chloro-4-(morpholin-4-yl)-phenylcarbamoyl]-2-methoxy-ethyl}-amide,
(3) 5-bromo-thiophene-2-carboxylic acid-N-{1-[3-chloro-4-([1,2]oxazinan-2-yl)-phenylcarbamoyl]-1-methyl-ethyl}-amide,
(4) 5-chloro-thiophene-2-carboxylic acid-N-{1-[3-methyl-4-([1,2]oxazinan-2-yl)-phenylcarbamoyl]-1-methyl-ethyl}-amide,
(5) 5-bromo-thiophene-2-carboxylic acid-N-{(1R)-1-[3-chloro-4-([1,2]oxazinan-2-yl)-phenylcarbamoyl]-2-methoxy-ethyl}-amide,
(6) 5-bromo-thiophene-2-carboxylic acid-N-{1-[3-chloro-4-([1,2]oxazepan-2-yl)-phenylcarbamoyl]-1-methyl-ethyl}-amide,
(7) 5-chloro-thiophene-2-carboxylic acid-N-{1-[3-methyl-4-([1,2]oxazepan-2-yl)-phenylcarbamoyl]-1-methyl-ethyl}-amide,
(8) 5-bromo-thiophene-2-carboxylic acid-N-{(1R)-1-[3-chloro-4-([1,2]oxazepan-2-yl)-phenylcarbamoyl]-2-methoxy-ethyl}-amide,

### Example 10

### 5-chloro-thiophene-2-carboxylic acid-N-{1-[3-chloro-4-(4-methyl-[1,4]diazepan-1-yl)-phenylcarbamoyl]-ethyl)amide

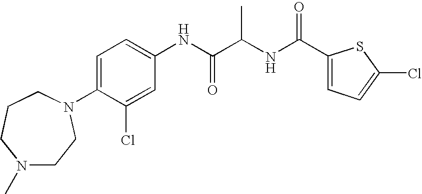

### (a) methyl 2-[(5-chloro-thiophene-2-carbonyl)-amino]-propionate

Prepared analogously to Example 1c from 5-chloro-thiophene-2-carboxylic acid and methyl 2-amino-propionate with TBTU and NMM in THF.

Yield: 81%

R_{f} value: 0.63 (silica gel; petroleum ether/ethyl acetate=1:1)

C₉H₁₀ClNO₃S (247.70)

Mass spectrum: (M+H)⁺=248/250 (chlorine isotope)

### (b) 2-[(5-chloro-thiophene-2-carbonyl)-amino]-propionic acid

3.80 g (15.3 mmol) methyl 2-[(5-chloro-thiophene-2-carbonyl)-amino]-propionate are stirred in 15.5 ml 1-molar sodium hydroxide solution and 15 ml of ethanol for 4 h at ambient temperature. Then the mixture is evaporated down i. vac., the residue is combined with ice water and extracted twice with diethyl ether. The aqueous phase is poured onto ice with acetic acid, the resulting precipitate is filtered off, washed with water and dried at 60° C.

Yield: 2.90 g (81%)

R_{f} value: 0.05-0.35 (silica gel; petroleum ether/ethyl acetate=1:2)

C₈H₈ClNO₃S (233.67)

### (c) 5-chloro-thiophene-2-carboxylic acid-N-f 1-[3-chloro-4-(4-methyl-[1,4]diazepan-1-yl)-phenyl-carbamoyl]-ethyl-amide

Prepared analogously to Example 1c from 2-[(5-chloro-thiophene-2-carbonyl)-amino]-propionic acid and 3-chloro-4-(4-methyl-[1,4]diazepan-1-yl)-aniline with TBTU and NMM in DMF.

Yield: 9%

Rₜ value: 4.08 min

C₂₀H₂₄Cl₂N₄O₂S*CF₃COOH (569.43/455.36)

Mass spectrum: (M+H)⁺=455/457/459 (chlorine isotope)

### Example 17

### 5-chloro-thiophene-2-carboxylic acid-N-{4-[3-chloro-4-(4-methyl-[1,4]diazepan-1-yl)-phenylcarbamoyl]-tetrahydropyran-4-yl}-amide

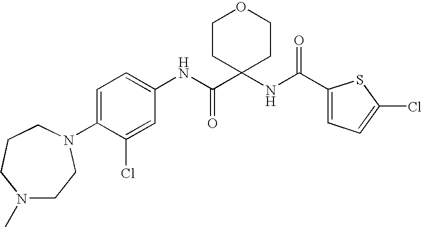

### (a) 4-Boc-amino-N-[3-chloro-4-(4-methyl-1,4]diazepan-1-yl)-phenyl]-tetrahydropyran-4-carboxylic acid-amide

Prepared analogously to Example 8 from 4-Boc-amino-tetrahydropyran-4-carboxylic acid and 3-chloro-4-(4-methyl-[1,4]diazepan-1-yl)-aniline with HATU and NMM in DMF.

Yield: 40%

Rₜ value: 2.31 min

C₂₃H₃₅ClN₄O₄ (467.00)

Mass spectrum: (M+H)⁺=467/469 (chlorine isotope)

### (b) 4-amino-N-[3-chloro-4-(4-methyl-[1.4]diazepan-1-yl)-Phenyl]-tetrahydropyran-4-carboxylic acid-amide

Prepared analogously to Example 1d from 4-Boc-amino-N-[3-chloro-4-(4-methyl-[1,4]diazepan-1-yl)-phenyl]-tetrahydropyran-4-carboxylic acid-amide with hydrochloric acid in dioxane.

Yield: quant.

Rₜ value: 1.72 min

C₁₈H₂₇ClN₄O₂*3HCl (476.27/366.89)

### (c) 5-chloro-thiophene-2-carboxylic acid-N-{1-[3-chloro-4-(4-methyl-[1,4]diazepan-1-yl)-Phenyl-carbamoyl]-tetrahydropyran-4-yl}-amide

Prepared analogously to Example 1c from 5-chloro-thiophene-2-carboxylic acid and 4-amino-N-[3-chloro-4-(4-methyl-[1,4]diazepan-1-yl)-phenyl]-tetrahydropyran-4-carboxylic acid-amide with TBTU and NMM in DMF.

Yield: 2%

Rₜ value: 2.45 min

C₂₃H₂₈Cl₂N₄O₃S*CF₃COOH (625.49/511.42)

Mass spectrum: (M+H)⁺=511/513 (chlorine isotope)

The following compounds were prepared analogously:

| | Structural formula | | | |
|---|---|---|---|---|
| No. | Name | Yield | Mass peak(s) | R_{f} value or Rₜ |
| 18 | 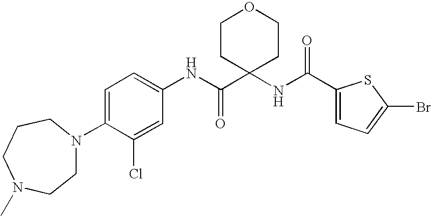 | Σ: 2.4% | (M + H)⁺ = 556/558/560 (bromine and chlorine isotopes) | 2.53 min |
| | 5-bromo-thiophene-2-carboxylic acid-N-{4-[3-chloro-4-(4-methyl-[1,4]diaze- | | | |
| | pan-1-yl)-phenylcarbamoyl]-tetrahydropyran-4-yl}-amide | | | |
| 19 | 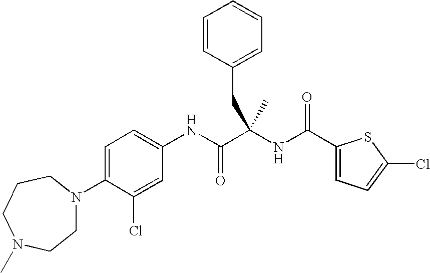 | Σ: 5.8% | (M + H)⁺ = 545/547/549 (chlorine isotope) | 0.13 (silica gel; dichloromethane/ methanol = 9:1) |
| | 5-chloro-thiophene-2-carboxylic acid-N-{(1R)-1-[3-chloro-4-(4-methyl-[1,4]diaze- | | | |
| | pan-1-yl)-phenylcarbamoyl]-1-methyl-2-phenyl-ethyl}-amide | | | |
| 20 | 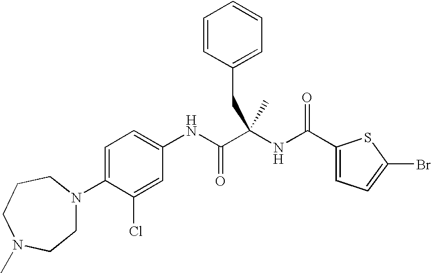 | Σ: 7.6% | (M + H)⁺ = 590/592/594 (bromine and chlorine isotopes) | 0.13 (silica gel; dichloromethane/ methanol = 9:1) |

5-bromo-thiophene-2-carboxylic acid-N-{(1R)-1-[3-chloro-4-(4-methyl-[1,4]diaze-
pan-1-yl)-phenylcarbamoyl]-1-methyl-2-phenyl-ethyl}-amide

### Example 21

### 5-bromo-thiophene-2-carboxylic acid-N-{1-[4-(4-methyl-[1,4]diazepan-1-yl)-phenylcarbamoyl)-1-methyl-ethyl}-amide

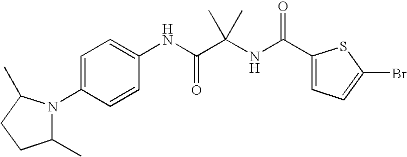

### (a) 4-(2,5-dimethyl-pyrrolidin-1-yl)-1-nitro-benzene

Prepared analogously to Example 1a from 4-fluoro-1-nitro-benzene and 2,5-dimethyl-pyrrolidine with potassium carbonate in DMSO.

Yield: 58%

C₁₂H₁₆N₂O₂ (220.27)

Mass spectrum: (M+H)⁺=221

### (b) 4-(2,5-dimethyl-pyrrolidin-1-yl)-aniline

250 mg (1.14 mmol) 4-(2,5-dimethyl-pyrrolidin-1-yl)-1-nitro-benzene in 10 ml of methanol are combined with 150 mg 10% palladium charcoal and 450 μl conc. hydrochloric acid and hydrogenated in a Parr apparatus at ambient temperature for 1.5 h at 50 psi hydrogen pressure. Then the palladium charcoal is filtered off and the filtrate is evaporated down i. vac. The residue is further reacted directly without any further purification.

Yield: 250 mg (50%)

C₁₂H₁₈N₂*2HCl (263.21/190.29)

Mass spectrum: (M+H)⁺=191

### (c) 2-Boc-amino-N-[4-(2,5-dimethyl-pyrrolidin-1-yl)-phenyl]-2-methyl-propionic acid-amide

Prepared analogously to Example 1c from 2-Boc-amino-isobutyric acid and 4-(2,5-dimethyl-pyrrolidin-1-yl)-aniline with TBTU and NMM in DMF.

Yield: 75%

R_{f} value: 0.60 (Alox; dichloromethane/methanol=95:5)

C₂₁H₃₃N₃O₃ (375.51)

Mass spectrum: (M+H)⁺=376

### (d) 2-amino-N-[4-(2,5-dimethyl-pyrrolidin-1-yl)-phenyl]-2-methyl-propionic acid-amide

Prepared analogously to Example 1d from 2-Boc-amino-N-[4-(2,5-dimethyl-pyrrolidin-1-yl)-phenyl]-2-methyl-propionic acid with hydrochloric acid in dioxane.

Yield: quant.

R_{f} value: 0.10 (Alox; dichloromethane/methanol=9:1)

C₁₆H₂₅N₃O*2HCl (348.31/275.39)

### (e) 5-bromo-thiophene-2-carboxylic acid-N-[1-(4-(2,5-dimethyl-pyrrolidin-1-yl)-phenylcarbamoyl)-1-methyl-ethyl]-amide

Prepared analogously to Example 1c from 5-bromo-thiophene-2-carboxylic acid and 2-amino-N-[4-(2,5-dimethyl-pyrrolidin-1-yl)-phenyl]-2-methyl-propionic acid-amide with TBTU and NMM in DMF.

Yield: 54%

Rₜ value: 2.51 min

C₂₁H₂₆BrN₃O₂S*CF₃COOH (578.44/464.38)

Mass spectrum: (M+H)⁺=464/466 (bromine isotope)

The following compound was prepared analogously:

| | Structural formula | | | |
|---|---|---|---|---|
| No. | Name | Yield | Mass peak(s) | R_{f} value or Rₜ |
| 22 | 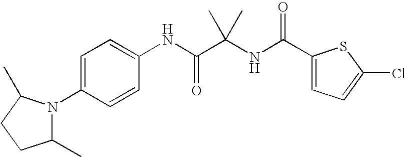 | Σ: 12.6% | (M + H)⁺ = 420/422 (chlorine isotope) | 2.50 min |

5-chloro-thiophene-2-carboxylic acid-N-{1-[4-(2,5-dimethyl-pyrrolidin-1-yl)-phenyl-
carbamoyl]-1-methyl-ethyl}-amide

### Example 23

### 5-bromo-thiophene-2-carboxylic acid-N-{1-[3-chloro-4-(4-methyl-[1,4]diazepan-1-yl)-phenylcarbamoyl]-cyclopent-1-yl}-amide

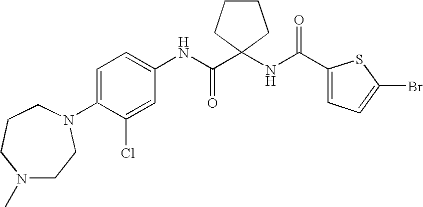

### (a) methyl 1-[(5-bromo-thiophene-2-carbonyl)-amino]-cyclopentyl-1-carboxylate

576 mg (2.78 mmol) 5-bromo-thiophene-2-carboxylic acid are combined with 5 ml of thionyl chloride in 5 ml dichloromethane and refluxed for 2 hours. The mixture is then evaporated down i. vac., combined with toluene and evaporated down completely. The residue is added dropwise in 5 ml dichloromethane to a mixture of 500 mg (2.78 mmol) methyl 1-amino-cyclopentyl-1-carboxylate and 1.94 ml (13.9 mmol) TEA in 5 ml dichloromethane with stirring and then stirred for 4 h at ambient temperature. The reaction mixture is extracted twice with water, the aqueous phases are re-extracted with dichloromethane and the combined organic phases are dried over magnesium sulphate and evaporated down completely.

Yield: 800 mg (87%)

R_{f} value: 0.91 (silica gel; dichloromethane/methanol=9:1)

C₁₂H₁₄BrNO₃S (332.22)

Mass spectrum: (M+H)⁺=332/334 (bromine isotope)

### (b) 1-[(5-bromo-thiophene-2-carbonyl)-amino]-cyclopentyl-1-carboxylic acid

Prepared analogously to Example 10b from methyl 1-[(5-bromo-thiophene-2-carbonyl)-amino]-cyclopentyl-1-carboxylate with 1-molar sodium hydroxide solution in methanol.

Yield: 52%

R_{f} value: 0.31 (silica gel; dichloromethane/methanol=9:1)

C₁₁H₁₂BrNO₃S (318.19)

Mass spectrum: (M+H)⁺=318/320 (bromine isotope)

### (c) 5-bromo-thiophene-2-carboxylic acid-N-f 1-[3-chloro-4-(4-methyl-[1,4]diazepan-1-yl)-phenyl-carbamoyl]-cyclopent-1-yl]-amide

Prepared analogously to Example 8 from 1-[(5-bromo-thiophene-2-carbonyl)-amino]-cyclopentyl-1-carboxylic acid and 3-chloro-4-(4-methyl-[1,4]diazepan-1-yl)-aniline with HATU and NMM in DMF.

Yield: 27%

R_{f} value: 0.29 (silica gel; dichloromethane/methanol=9:1)

C₂₃H₂₈BrClN₄O₂S (539.92)

Mass spectrum: (M+H)⁺=539/541/543 (bromine and chlorine isotopes)

### Example 24

### 5-chloro-thiophene-2-carboxylic acid-N-{1-[4-(4-methyl-piperazin-1-yl)-phenylcarbamoyl)-1-methyl-ethyl}-amide

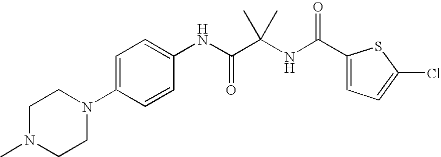

### (a) 2-(5-chloro-thiophen-2-yl)-4,4-dimethyl-4H-oxazol-5-one

1.00 g (4.04 mmol) 2-([5-chloro-thiophene-2-carbonyl]-amino)-isobutyric acid are heated to 85° C. in 30 ml acetic anhydride for 1 h and then evaporated down completely.

Yield: 927 mg (quant.)

C₉H₈NO₂S (229.68)

Mass spectrum: (M+H)⁺=229/231 (chlorine isotope)

### (b) 4-(2,5-dimethyl-pyrrolidin-1-yl)-aniline

200 μl of a 0.05-molar solution of 2-(5-chloro-thiophen-2-yl)-4,4-dimethyl-4H-oxazol-5-one in a solvent mixture of toluene and glacial acetic acid 9:1 are combined with 200 μl of a 0.05-molar solution of 4-(4-methyl-piperazin-1-yl)-aniline in DMF with 5% DIPEA and heated to 80° C. for 16 h. The mixture is then left to stand open for 7 days, then filtered through basic aluminium oxide and washed again with DMF/methanol 9:1. It is then concentrated by evaporation i. vac.

Yield: 5.1 mg (quant.)

Rₜ value: 3.18 min

C₂₀H₂₅ClN₄O₂S (420.96)

Mass spectrum: (M+H)⁺=421/423 (chlorine isotope)

The following compound was prepared analogously:

| | Structural formula | | | |
|---|---|---|---|---|
| No. | Name | Yield | Mass peak(s) | R_{f} value or Rₜ |
| 25 | 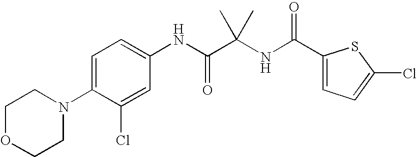 | Σ: quant. | (M + H)⁺ = 442/444/446 (chlorine isotope) | 4.14 min |

5-chloro-thiophene-2-carboxylic acid-N-{3-chloro-1-[4-(morpholin-4-yl)-phenyl-
carbamoyl]-1-methyl-ethyl}-amide

### Example 27

### 5-bromo-thiophene-2-carboxylic acid-N-{1-methyl-1-[3-chloro-4-(2-methyl-tetrahydropyridazin-1-yl)-phenylcarbamoyl)-ethyl}-amide

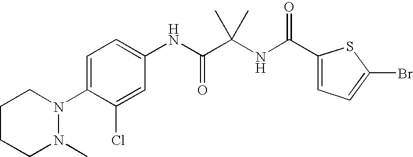

### (a) N-Boc-N-methyl-hydrazine

4.47 ml (82.3 mmol) methylhydrazine are added dropwise to a mixture of 17.07 g (78.23 mmol) di-tert.-butyl pyrocarbonate in 75 ml of methanol with stirring at ambient temperature, refluxed for 2.5 hours and then evaporated down i. vac. The residue is further reacted directly without any further purification.

Yield: 10.50 g (92%)

C₆H₁₄N₂O₂ (146.19)

Mass spectrum: (M+H)⁺=147

### (b) N-Boc-N′-(2-chloro-4-nitro-phenyl)-N-methyl-hydrazine

2.30 g (13.1 mmol) 3-chloro-4-fluoro-1-nitro-benzene are stirred together with 4.50 g (26.2 mmol) N-Boc-N-methyl-hydrazine and 7.30 g (52.8 mmol) potassium carbonate in 25 ml DMF for 3 days at ambient temperature, then for 4 h at 90° C. and then 16 h at ambient temperature. After evaporation of the reaction mixture i. vac. it is combined with a mixture of water/ethyl acetate 1:1, adjusted to pH 4 with 0.5-normal potassium hydrogen sulphate solution and extracted with ethyl acetate. The combined organic phases are washed with semisat. and sat. sodium chloride solution, dried over magnesium sulphate and evaporated down i. vac. The residue is combined with cyclohexane and triturated. After filtration it is washed with cyclohexane and dried at 55° C.

Yield: 1.41 g (36%)

R_{f} value: 0.38 (silica gel; cyclohexane/ethyl acetate=8:2)

C₁₂H₁₆ClN₃O₄ (301.73)

Mass spectrum: (M+H)⁺=301/303 (chlorine isotope)

### (c) N-Boc-N′-(4-bromo-butyl)-N′-(2-chloro-4-nitro-phenyl)-N-methyl-hydrazine

350 mg (1.16 mmol) N-Boc-N′-(2-chloro-4-nitro-phenyl)-N-methyl-hydrazine are combined together with 350 μl (2.93 mmol) 1,4-dibromobutane in 7.5 ml DMF in a Schlenck tube under a nitrogen atmosphere and while being cooled in the ice bath with 50.6 mg (1.16 mmol) 55% sodium hydride, dispersed in paraffin oil. After 10 min cooling in the ice bath, 30 min stirring at ambient temperature and heating to 75° C. for 1 h the reaction mixture is evaporated down completely i. vac. and the residue is combined twice with toluene and dichloromethane and evaporated down completely. The residue is further reacted directly without any further purification.

Yield: 900 mg (approx. 53%, contaminated)

R_{f} value: 0.69 (silica gel; cyclohexane/ethyl acetate=6:4+0.5% conc. ammonia solution)

C₁₆H₂₃BrClN₃O₄ (436.73)

### (d) 3-chloro-4-(2-methyl-tetrahydropyridazin-1-yl)-1-nitro-benzene

890 mg (approx. 0.71 mmol) N-Boc-N′-(4-bromo-butyl)-N′-(2-chloro-4-nitro-phenyl)-N-methyl-hydrazine (product obtained above) are combined with 1.50 ml (19.6 mmol) TFA in 10 ml dichloromethane at ambient temperature with stirring and then stirred for 90 min at ambient temperature. After evaporation i. vac. the residue is taken up in dichloromethane 3 times and evaporated down completely. Then the residue is taken up in 10 ml acetone, combined with 490 mg (3.55 mmol) potassium carbonate and stirred for 15 h at ambient temperature. After filtration the filtrate is evaporated down i. vac. and the residue is purified by chromatography on silica gel (eluant gradient: cyclohexane/ethyl acetate=9:1->8:2).

Yield: 180 mg (quant.)

R_{f} value: 0.48 (silica gel; cyclohexane/ethyl acetate=8:2)

C₁₁H₁₄ClN₃O₂ (255.70)

Mass spectrum: (M+H)⁺=255/257 (chlorine isotope)

### (e) 3-chloro-4-(2-methyl-tetrahydropyridazin-1-yl)-aniline

Prepared analogously to Example 1b from 3-chloro-4-(2-methyl-tetrahydropyridazin-1-yl)-1-nitro-benzene by hydrogenation with hydrogen and Raney nickel in ethyl acetate.

Yield: 63% (contaminated)

R_{f} value: 0.71 (RP-8; 5%-ige sodium chloride solution/methanol=2:3

C₁₁H₁₆ClN₃*2HCl (298.64/225.72)

Mass spectrum: (M+H)⁺=225/227 (chlorine isotope)

### (f) 5-bromo-thiophene-2-carboxylic acid-N-[1-methyl-1-[3-chloro-4-(2-methyl-tetrahydropyridazin-1-yl)-phenylcarbamoyl]-ethyl]-amide

Prepared analogously to Example 8 from 3-chloro-4-(2-methyl-tetrahydropyridazin-1-yl)-aniline and 2-[(5-bromo-thiophene-2-carbonyl)-amino]-isobutyric acid with HATU and NMM in NMP.

Yield: 38% (HPLC)

Rₜ value: 3.25 min

C₂₀H₂₄BrClN₄O₂S (499.85)

Mass spectrum: (M+H)⁺=499/501/503 (bromine and chlorine isotopes)

The following compounds may be prepared analogously:
(1) 5-chloro-thiophene-2-carboxylic acid-N-{1-[3-methyl-4-(2-methyl-tetrahydropyridazin-1-yl)-phenylcarbamoyl]-1-methyl-ethyl}-amide,
(2) 5-bromo-thiophene-2-carboxylic acid-N-{(1R)-1-[3-chloro-4-(2-methyl-tetrahydropyridazin-1-yl)-phenylcarbamoyl]-2-methoxy-ethyl}-amide,
(3) 5-bromo-thiophene-2-carboxylic acid-N-{3-[3-methyl-4-(2-methyl-tetrahydro-pyridazin-1-yl)-phenylcarbamoyl]-tetrahydrofuran-3-yl}-amide,
(4) 5-chloro-thiophene-2-carboxylic acid-N{3-[3-methyl-4-(2-methyl-tetrahydro-pyridazin-1-yl)-phenylcarbamoyl]-tetrahydrofuran-3-yl}-amide,
(5) 5-chloro-thiophene-2-carboxylic acid-N-2-[4-(2-methyl-tetrahydro-pyridazin-1-yl)-phenylcarbamoyl]-bicyclo[2.2.1]hept-2-ylamide,
(6) 5-chloro-thiophene-2-carboxylic acid-N-3-[3-chloro-4-(2-methyl-tetrahydro-pyridazin-1-yl)-phenylcarbamoyl]-tetrahydrofuran-3-yl}-amide,
(7) 5-chloro-thiophene-2-carboxylic acid-N-3-[3-bromo-4-(2-methyl-tetrahydro-pyridazin-1-yl)-phenylcarbamoyl]-tetrahydrofuran-3-yl}-amide,
(8) 5-chloro-thiophene-2-carboxylic acid-N-{3-[3-fluoro-4-(2-methyl-tetrahydro-pyridazin-1-yl)-phenylcarbamoyl]-tetrahydrofuran-3-yl}-amide,
(9) 5-chloro-thiophene-2-carboxylic acid-N-{3-[3-trifluoromethyl-4-(2-methyl-tetrahydro-pyridazin-1-yl)-phenylcarbamoyl]-tetrahydrofuran-3-yl}-amide,
(10) 5-chloro-thiophene-2-carboxylic acid-N-{3-[4-(2-methyl-tetrahydro-pyridazin-1-yl)-phenylcarbamoyl]-tetrahydrofuran-3-yl}-amide,
(11) 5-chloro-thiophene-2-carboxylic acid-N-{2-[3-methyl-4-(2-methyl-tetrahydro-pyridazin-1-yl)-phenylcarbamoyl]-bicyclo[2.2.1]hept-2-yl}-amide,
(12) 5-chloro-thiophene-2-carboxylic acid-N-{2-[3-methyl-4-(2-methyl-tetrahydro-pyridazin-1-yl)-phenylcarbamoyl]-7-oxa-bicyclo[2.2.1]hept-2-yl}-amide,
(13) 5-chloro-thiophene-2-carboxylic acid-N-{3-[3-methyl-4-([2-methyl-[1,2]diazepan-1-yl)-phenylcarbamoyl]-tetrahydrofuran-3-yl}-amide,
(14) 5-chloro-thiophene-2-carboxylic acid-N-{3-[3-methyl-4-(5-methyl-[1,4,5]oxadiazepan-4-yl)-phenylcarbamoyl]-tetrahydrofuran-3-yl}-amide,
(15) 5-bromo-thiophene-2-carboxylic acid-N-{3-[4-(5-methyl-[1,4,5]oxadiazepan-4-yl)-phenylcarbamoyl]-tetrahydrofuran-3-yl}-amide.

The Examples that follow describe the preparation of pharmaceutical formulations which contain as active substance any desired compound of general formula (I):

### Example I

Dry ampoule containing 75 mg of active substance per 10 ml
Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the
solution is freeze-dried. To produce the solution ready for use for
injections, the product is dissolved in water.

### Example II

Dry ampoule containing 35 mg of active substance per 2 ml
Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging,
the solution is freeze-dried.
To produce the solution ready for use for injections, the product is
dissolved in water.

### Example III

Tablet containing 50 mg of active substance
Composition:

| | | |
|---|---|---|
| (1) Active substance | 50.0 | mg |
| (2) Lactose | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

Preparation:

(1), (2) and (3) are mixed together and granulated with an aqueous
solution of (4). (5) is added to the dried granulated material.
From this mixture tablets are pressed, biplanar, faceted on both
sides and with a dividing notch on one side. Diameter of the
tablets: 9 mm.

### Example IV

Tablet containing 350 mg of active substance
Composition:

| | | |
|---|---|---|
| (1) Active substance | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

Preparation:

(1), (2) and (3) are mixed together and granulated with an aqueous
solution of (4). (5) is added to the dried granulated material.
From this mixture tablets are pressed, biplanar, faceted on both
sides and with a dividing notch on one side. Diameter of the
tablets: 12 mm.

### Example V

Capsules containing 50 mg of active substance
Composition:

| | | |
|---|---|---|
| (1) Active substance | 50.0 | mg |
| (2) Dried maize starch | 58.0 | mg |
| (3) Powdered lactose | 50.0 | mg |
| (4) Magnesium stearate | 2.0 | mg |
| | 160.0 | mg |

Preparation:

(1) is triturated with (3). This trituration is added to the mixture of
(2) and (4) with vigorous mixing.

This powder mixture is packed into size 3 hard gelatine capsules in a capsule filling machine.

### Example VI

Capsules containing 350 mg of active substance
Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

Preparation:

(1) is triturated with (3). This trituration is added to the mixture of
(2) and (4) with vigorous mixing.

This powder mixture is packed into size 0 hard gelatine capsules in a capsule filling machine.

### Example VII

Suppositories containing 100 mg of active substance
1 suppository contains:

| | | |
|---|---|---|
| Active substance | 100.0 | mg |
| Polyethyleneglycol (M.W. 1500) | 600.0 | mg |
| Polyethyleneglycol (M.W. 6000) | 460.0 | mg |
| Polyethylenesorbitan monostearate | 840.0 | mg |
| | 2,000.0 | mg |

Preparation:

The polyethyleneglycol is melted together with polyethylenesorbitan
monostearate. At 40° C. the ground active substance is homogeneously
dispersed in the melt. It is cooled to 38° C. and poured into slightly chilled
suppository moulds.

1. A compound of the formula 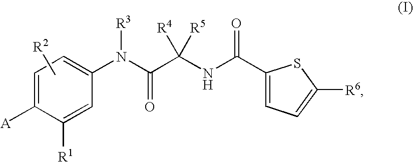
wherein:
A denotes a group of the formula 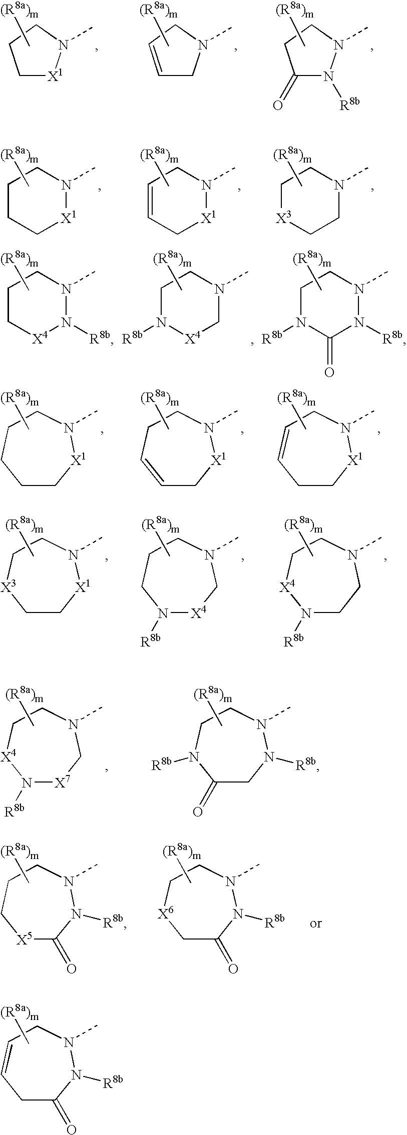
wherein
m is the number 1 or 2,
R^{8a} in each case independently of one another denotes a hydrogen or fluorine atom or a C₁₋₅-alkyl, hydroxy, hydroxy-C₁₋₅-alkyl, C₁₋₅-alkoxy, C₁₋₅-alkoxy-C₁₋₅-alkyl, amino, C₁₋₅-alkylamino, di-(C₁₋₅-alkyl)-amino, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl or C₁₋₅-alkylcarbonylamino group, while
in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} in each case independently of one another denotes a hydrogen atom or a C₁₋₅-alkyl group,
X¹ denotes an oxygen atom or a —CH₂—, —CHR^{8a}— or —NR^{8c}— group,
R^{8c} in each case independently of one another denotes a hydrogen atom, a C₁₋₅-alkyl, C₁₋₅-alkylcarbonyl, C₁₋₅-alkyloxycarbonyl or C₁₋₅-alkylsulphonyl group,
X² denotes an oxygen atom or a —NR^{8b}— group,
X³ denotes an oxygen or sulphur atom, a —NR^{8c}— group,
X⁴ denotes a carbonyl or sulphonyl group,
X⁵ denotes an oxygen atom, a —NR^{8b}— or methylene group,
X⁶ denotes an oxygen or sulphur atom or a —NR^{8c}— group,
X⁷ denotes a methylene or carbonyl group,
R¹ denotes a hydrogen, fluorine, chlorine, bromine or iodine atom, a C₁₋₃-alkyl or C₁₋₃-alkoxy group, while the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R³ denotes a hydrogen atom or a C₁₋₃-alkyl group,
R⁴ and R⁵ in each case independently of one another denote
a hydrogen atom, a C₂₋₆-alkenyl or C₂₋₆-alkynyl group,
a straight-chain or branched C₁₋₆-alkyl group,
while the hydrogen atoms of the straight-chain or branched C₁₋₆-alkyl group may optionally be wholly or partly replaced by fluorine atoms, and may optionally be substituted by a C₃₋₅-cycloalkyl group, a nitrile, hydroxy, a C₁₋₅-alkyloxy group, while the hydrogen atoms of the C₁₋₅-alkyloxy group may optionally be wholly or partly replaced by fluorine atoms, an allyloxy, propargyloxy, benzyloxy, C₁₋₅-alkylcarbonyloxy, C₁₋₅-alkyloxycarbonyloxy, carboxy-C₁₋₅-alkyloxy, C₁₋₅-alkyloxycarbonyl-C₁₋₅-alkyloxy, mercapto, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino group,
a carboxy, aminocarbonyl, C₁₋₅-alkylaminocarbonyl, C₃₋₆-cycloalkylaminocarbonyl, di-(C₁₋₅-alkyl)-aminocarbonyl, C₁₋₅-alkoxycarbonyl, C₄₋₆-cycloalkyleneiminocarbonyl group,
a phenyl, heteroaryl, phenyl-C₁₋₅-alkyl or heteroaryl-C₁₋₅-alkyl group,
which may optionally be mono- to trisubstituted in the phenyl or heteroaryl moiety by identical or different substituents selected from the group consisting of halogen atoms, C₁₋₅-alkyl, di-(C₁₋₅-alkyl)-amino, hydroxy, C₁₋₅-alkyloxy, mono-, di- or trifluoromethoxy, carboxy- and C₁₋₅-alkyloxycarbonyl groups,
a 3- to 7-membered cycloalkyl, cycloalkyleneimino, cycloalkyl-C₁₋₅-alkyl or cycloalkyleneimino-C₁₋₃-alkyl group,
wherein in 4- to 7-membered cyclic groups in the cyclic moiety a methylene group may optionally be replaced by a —N(R^{8c})— group, an oxygen or sulphur atom or a —S(O)— or —S(O)₂— group, or
wherein in 4- to 7-membered cyclic groups in the cyclic moiety two adjacent methylene groups together may optionally be replaced by a —C(O)N(R^{8b})— or —S(O)₂N(R^{8b})— group, or
wherein in 6- to 7-membered cyclic groups in the cyclic moiety three adjacent methylene groups together may optionally be replaced by a substituted —OC(O)N(R^{8b})— or —N(R^{8b})C(O)N(R^{8b})— or
—N(R^{8b})S(O)₂N(R^{8b})— group,
with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms from the group oxygen and nitrogen are separated from one another by precisely one optionally substituted —CH₂— group, is excluded,
while a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two C₁₋₃-alkyl groups in each case, or
R⁴ and R⁵ together with the carbon atom to which they are bound form a C₃₋₈-cycloalkyl or C₃₋₈-cycloalkenyl group,
while a C₃₋₈-cycloalkyl or C₄₋₈-cycloalkenyl group may be substituted at an individual carbon atom by a C₂₋₅-alkylene group or simultaneously at two different carbon atoms by a C₁₋₄-alkylene group forming a corresponding spirocyclic group or a bridged bicyclic group,
while one of the methylene groups of a C₄₋₈-cycloalkyl or C₅₋₈-cycloalkenyl group or a corresponding spirocyclic group as described above or a corresponding bridged bicyclic group may be replaced by an oxygen or sulphur atom or a —N(R^{8c})—, or a carbonyl, sulphinyl or sulphonyl group, and/or
two directly adjacent methylene groups of a C₄₋₈-cycloalkyl group together by a —C(O)N(R^{8b})—, —C(O)O— or —S(O)₂N(R^{8b})— group may be replaced, and/or
three directly adjacent methylene groups of a C₆₋₈-cycloalkyl group may together be replaced by a —OC(O)N(R^{8b})—, —N(R^{8b})C(O)N(R^{8b})— or —N(R^{8b})S(O)₂N(R^{8b})— group,
while 1 to 3 carbon atoms of a C₃₋₈-cycloalkyl group or a corresponding spirocyclic group as described above or a corresponding bridged bicyclic group may optionally be substituted independently of one another in each case by one or two fluorine atoms or one or two identical or different C₁₋₅-alkyl, nitrile, hydroxy, C₁₋₅-alkyloxy, C₁₋₅-alkylcarbonyloxy, carboxy-C₁₋₅-alkyl, C₁₋₅-alkyloxycarbonyl-C₁₋₅-alkyl, C₁₋₅-alkylsulphanyl, C₁₋₅-alkylsulphonyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₄-cycloalkyleneiminosulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
while 1 to 2 carbon atoms of a C₃₋₈-cycloalkenyl group may optionally be substituted independently of one another in each case by a C₁₋₅-alkyl, nitrile, carboxy-C₁₋₅-alkyl, C₁₋₅-alkyloxycarbonyl-C₁₋₅-alkyl, carboxy, C₁₋₅-alkyloxycarbonyl, aminocarbonyl, C₁₋₅-alkylaminocarbonyl, di-(C₁₋₅-alkyl)-aminocarbonyl, C₃₋₆-cycloalkyleneiminocarbonyl, aminosulphonyl, C₁₋₅-alkylaminosulphonyl, di-(C₁₋₅-alkyl)-aminosulphonyl, C₃₋₆-cycloalkyleneiminosulphonyl groups,
and 1 to 2 carbon atoms of a C₄₋₈-cycloalkenyl group which are not bound to another carbon atom by a double bond, may optionally be substituted independently of one another by one or two fluorine atoms or a hydroxy, C₁₋₅-alkyloxy, C₁₋₅-alkylcarbonyloxy, C₁₋₅-alkylsulphanyl, C₁₋₅-alkylsulphonyl, amino, C₁₋₅-alkylamino, di-(C₁₋₅-alkyl)-amino, C₁₋₅-alkylcarbonylamino, C₁₋₅-alkylsulphonylamino, N-(C₁₋₅-alkylsulphonyl)-C₁₋₅-alkylamino or C₃₋₆-cycloalkylcarbonylamino groups,
with the proviso that a C₃₋₈-cycloalkyl or C₃₋₈-cycloalkenyl group of this kind formed from R⁴ and R⁵ together or a corresponding spirocyclic group as described above or a corresponding bridged bicyclic group,
wherein two heteroatoms in the cyclic group selected from among oxygen and nitrogen are separated from one another by precisely one optionally substituted —CH₂— group, and/or
wherein one or both methylene groups of the cyclic group which are linked directly to the carbon atom to which the groups R⁴ and R⁵ are bound are replaced by a heteroatom selected from among oxygen, nitrogen and sulphur, and/or
wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one, optionally substituted, methylene group, and/or
wherein two oxygen atoms are directly joined together, and/or
wherein a heteroatom selected from among oxygen, nitrogen and sulphur is linked directly to a carbon atom which is linked to another carbon atom by a double bond, and/or
which contains a cyclic group with three ring members, one or more of which correspond to an oxygen or sulphur atom or —N(R^{8c})— group,
is excluded,
R⁶ denotes a hydrogen, fluorine, chlorine, bromine or iodine atom, a nitrile group, a C₁₋₃-alkyl group, or a C₁₋₃-alkoxy group, while the hydrogen atoms of the C₁₋₃-alkyl or C₁₋₃-alkoxy group may optionally be wholly or partly replaced by fluorine atoms,
while, unless otherwise stated, by the term “heteroaryl group” mentioned hereinbefore in the definitions is meant a monocyclic 5- or 6-membered heteroaryl group, while
the 6-membered heteroaryl group contains one, two or three nitrogen atoms and
the 5-membered heteroaryl group contains an imino group optionally substituted by a C₁₋₃-alkyl, phenyl or phenyl-C₁₋₃-alkyl group, or an oxygen or sulphur atom or
an imino group optionally substituted by a C₁₋₃-alkyl, phenyl, amino-C₂₋₃-alkyl, C₁₋₃-alkylamino-C₂₋₃-alkyl, di-(C₁₋₃-alkyl)-amino-C₂₋₃-alkyl, a C₃₋₆-cycloalkyleneimino-C₁₋₃-alkyl or phenyl-C₁₋₃-alkyl group, or an oxygen or sulphur atom and additionally a nitrogen atom or
an imino group optionally substituted by a C₁₋₃-alkyl or phenyl-C₁₋₃-alkyl group, and two or three nitrogen atoms,
and moreover a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms
and the bond is effected via a nitrogen atom or a carbon atom of the heterocyclic moiety or a fused-on phenyl ring,
while, unless otherwise stated, by the term “halogen atom” mentioned hereinbefore in the definitions is meant an atom selected from among fluorine, chlorine, bromine and iodine,
while the alkyl, alkenyl, alkynyl and alkoxy groups contained in the foregoing definitions which have more than two carbon atoms, unless otherwise stated, may be straight-chain or branched and the alkyl groups in the previously mentioned dialkylated groups, for example the dialkylamino groups, may be identical or different,
and the hydrogen atoms of the methyl or ethyl groups contained in the foregoing definitions may, unless otherwise stated, be wholly or partly replaced by fluorine atoms,
or a tautomer or salt thereof.

2. A compound of the formula I according to claim 1, wherein:
A denotes a group of general formula 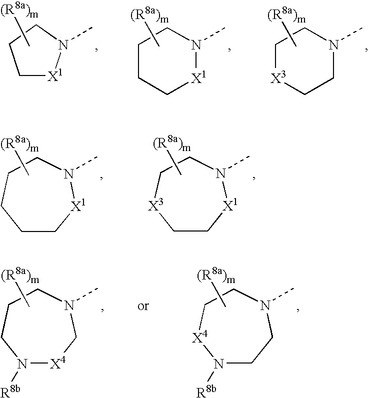
wherein
m is the number 1 or 2,
R^{8a} in each case independently of one another denotes a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl group, while in the previously mentioned substituted 5- to 7-membered groups A the heteroatoms F, O or N optionally introduced with R^{8a} as substituents are not separated by precisely one carbon atom from a heteroatom selected from among N, O, S, and two substituents R^{8a} on the same or different carbon atoms may denote a C₁₋₅-alkylene group,
R^{8b} denotes a hydrogen atom or a C₁₋₃-alkyl group,
X¹ denotes an oxygen atom or a —CH₂—, —CHR^{8a}— or —NR^{8c}— group,
R^{8c} in each case independently of one another denotes a hydrogen atom, a C₁₋₃-alkyl, C₁₋₃-alkylcarbonyl, or a C₁₋₄-alkyloxycarbonyl group,
X³ denotes an oxygen atom or a —NR^{8c}— group,
X⁴ denotes a carbonyl group,
R¹ denotes a fluorine, chlorine, bromine or iodine atom, a methyl or a methoxy group, while the hydrogen atoms of the methyl or methoxy group may optionally be wholly or partly replaced by fluorine atoms,
R² denotes a hydrogen or fluorine atom,
R³ denotes a hydrogen atom,
R⁴ denotes a straight-chain or branched C₁₋₄-alkyl group,
while the hydrogen atoms may optionally be wholly or partly replaced by fluorine atoms, and may optionally be substituted by a hydroxy, a C₁₋₃-alkyloxy group, while the hydrogen atoms of the C₁₋₃-alkyloxy group may be wholly or partly replaced by fluorine atoms, a benzyloxy, C₁₋₃-alkylcarbonyloxy, C₁₋₃-alkyloxycarbonyl, aminocarbonyl, C₁₋₃-alkylaminocarbonyl, di-(C₁₋₃-alkyl)-aminocarbonyl, C₃₋₆-cycloalkyleneiminocarbonyl, aminosulphonyl, C₁₋₃-alkylaminosulphonyl, di-(C₁₋₃-alkyl)-aminosulphonyl, C₃₋₆-cycloalkyleneiminosulphonyl, amino, C₁₋₃-alkylamino, di-(C₁₋₃-alkyl)-amino, C₁₋₃-alkylcarbonylamino, or C₁₋₃-alkylsulphonylamino group,
a heteroaryl-C₁₋₂-alkyl or C-linked heteroaryl group
while the heteroaryl group is selected from among pyrrolyl, oxazolyl, imidazolyl, furanyl, thiophenyl, thiazolyl, 1,2,3-triazolyl, 1,2,4-triazolyl, tetrazolyl, pyridinyl, pyrimidinyl and pyrazinyl, and may optionally be mono- to disubstituted in the heteroaryl moiety by identical or different substituents selected from among halogen atoms, C₁₋₃-alkyl, hydroxy, C₁₋₃-alkyloxy, mono-, di- and trifluoromethoxy groups,
R⁵ denotes a hydrogen atom,
a straight-chain or branched C₁₋₄-alkyl group,
while the hydrogen atoms may optionally be wholly or partly replaced by fluorine atoms, and may optionally be substituted by a C₁₋₃-alkyloxy group, while the hydrogen atoms of the C₁₋₃-alkyloxy group may optionally be wholly or partly replaced by fluorine atoms, or
R⁴ and R⁵ together with the carbon atom to which they are bound form a C₃₋₇-cycloalkyl or C₄₋₇-cycloalkenyl group,
while the C₃₋₇-cycloalkyl or C₄₋₇-cycloalkenyl group may be substituted at an individual carbon atom by a C₂₋₅-alkylene group or may be substituted simultaneously at two different carbon atoms by a C₁₋₄-alkylene group forming a corresponding spirocyclic group or a bridged bicyclic group,
while one of the methylene groups of a C₄₋₇-cycloalkyl or C₄₋₇-cycloalkenyl group or a corresponding spirocyclic group as described above or a corresponding bridged bicyclic group may be replaced by an oxygen or sulphur atom or a sulphonyl or —N(R^{8c})— group, and/or
two directly adjacent methylene groups of a C₄₋₈cycloalkyl group may together be replaced by a —C(O)N(R^{8b})— or —C(O)O— group,
while 1 to 2 carbon atoms of a C₃₋₇-cycloalkyl group or a corresponding spirocyclic group as described above or a corresponding bridged bicyclic group may optionally be substituted independently of one another by a C₁₋₃-alkyl, hydroxy, C₁₋₃-alkyloxy, di-(C₁₋₃-alkyl)-amino group,
with the proviso that a C₃₋₇cycloalkyl or C₄₋₇-cycloalkenyl group of this kind formed from R⁴ and R⁵ together or a corresponding spirocyclic group as described above or a corresponding bridged bicyclic group,
wherein methylene groups of the cyclic group which are directly connected to the carbon atom to which the groups R⁴ and R⁵ are bound, are replaced by a heteroatom selected from among oxygen, nitrogen and sulphur, and/or
wherein a substituent bound to the cyclic group, which is characterised in that a heteroatom selected from among oxygen and nitrogen is bound directly to the cyclic group, is separated from another heteroatom selected from among oxygen, nitrogen and sulphur, with the exception of the sulphone group, by precisely one, optionally substituted, methylene group, and/or
wherein a heteroatom selected from among oxygen, nitrogen and sulphur is linked directly to a carbon atom, which is linked to another carbon atom by a double bond, and/or
which contains a cyclic group with three ring members, of which one or more corresponds to an oxygen or sulphur atom or an —N(R^{8c})— group,
is excluded,
R⁶ denotes a chlorine or bromine atom,
while, unless otherwise stated, by the term “halogen atom” mentioned hereinbefore in the definitions is meant an atom selected from among fluorine, chlorine, bromine and iodine,
while, unless otherwise stated, the alkyl and alkoxy groups contained in the foregoing definitions which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
or a tautomer or salt thereof.

3. A compound of the formula I according to claim 1, wherein:
A denotes a group of general formula 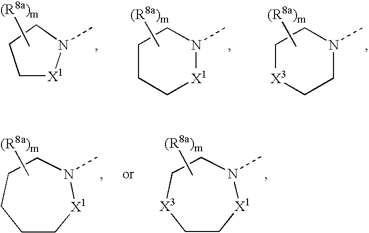
wherein
m is the number 1 or 2,
R^{8a} each independently of one another denote a hydrogen or fluorine atom or a C₁₋₃-alkyl, hydroxy, hydroxy-C₁₋₃-alkyl, C₁₋₃-alkoxy, C₁₋₃-alkoxy-C₁₋₃-alkyl, amino, C₁₋₃-alkylamino, di-(C₁₋₃-alkyl)-amino, amino-C₁₋₃-alkyl, C₁₋₃-alkylamino-C₁₋₃-alkyl, di-(C₁₋₃-alkyl)-amino-C₁₋₃-alkyl group, while in the previously mentioned substituted 5- to 7-membered groups A the heteroatoms F, O or N optionally introduced with R^{8a} as substituent are not separated by precisely one carbon atom from a heteroatom selected from among N, O, S,
R^{8b} denotes a hydrogen atom or a C₁₋₃-alkyl group,
X¹ denotes an oxygen atom or a —CH₂—, —CHR^{8a}— or —NR^{8c}— group,
R^{8c} denotes a hydrogen atom, a C₁₋₃-alkyl, C₁₋₃-alkylcarbonyl, or a C₁₋₄-alkyloxycarbonyl group,
X³ denotes an oxygen atom or a —NR^{8c}— group,
R¹ denotes a chlorine or bromine atom, a methyl, trifluoromethyl or a methoxy group,
R² denotes a hydrogen or fluorine atom,
R³ denotes a hydrogen atom,
R⁴ denotes a methyl group which may optionally be substituted by a hydroxy, methoxy, benzyloxy, methoxycarbonyl or pyridin-4-yl group, or
a furan-2-yl, 1-methyl-pyrazin-3-yl, phenyl, pyridin-3-yl or pyrazin-2-yl group,
R⁵ denotes a hydrogen atom or a methyl group, or
R⁴ and R⁵ together with the carbon atom to which they are bound form a C₃₋₆-cycloalkyl or C₅₋₆-cycloalkenyl group,
while the C₅₋₆-cycloalkyl or C₅₋₆-cycloalkenyl group may be substituted at a single carbon atom by a C₂₋₄-alkylene group or simultaneously at two different carbon atoms by a C₁₋₃-alkylene group, forming a corresponding spirocyclic group or a bridged bicyclic group,
while one of the methylene groups of a C₄₋₆-cycloalkyl or C₅₋₆-cycloalkenyl group or of a corresponding spirocyclic group or a corresponding bridged bicyclic group as described above, may be replaced by an oxygen atom or an —N(R^{8c})— group,
with the proviso that a C₃₋₆-cycloalkyl or C₅₋₆-cycloalkenyl group of this kind, formed from R⁴ and R⁵ together or a corresponding spirocyclic group or a corresponding bridged bicyclic group as described above,
wherein methylene groups of the cyclic group which are directly connected to the carbon atom to which the groups R⁴ and R⁵ are bound, are replaced by a heteroatom selected from among oxygen and nitrogen, and/or
wherein a heteroatom selected from among oxygen and nitrogen is linked directly to a carbon atom, which is linked to another carbon atom by a double bond, and/or
which contains a cyclic group with three ring members, of which one or more corresponds to an oxygen atom or —N(R^{8c})— group,
is excluded,
R⁶ denotes a chlorine or bromine atom
while, unless otherwise stated, by the term “halogen atom” mentioned hereinbefore in the definitions is meant an atom selected from among fluorine, chlorine, bromine and iodine,
while, unless otherwise stated, the alkyl and alkoxy groups contained in the foregoing definitions, which have more than two carbon atoms may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless otherwise stated, may be wholly or partly replaced by fluorine atoms,
or a tautomer or salt thereof.

4. A compound of the formula I according to claim 1, wherein R⁴ and R⁵ do not denote hydrogen.

5. A compound of the formula I according to claim 1 wherein R⁴ and R⁵ together with the carbon atom to which they are bound form a cyclic group.

6. A compound of the formula I according to claim 1 wherein R⁴ and R⁵ together with the carbon atom to which they are bound form a cyclic group, while in the cyclic group a methylene group is replaced by an oxygen atom or an —N(R^{8c})— group.

7. A compound of the formula I according to claim 1 wherein R⁴ and R⁵ together with the carbon atom to which they are bound denote a cyclic group of the formula

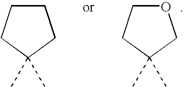

8. A compound of the formula I according to claim 1 wherein R⁴ and R⁵ together with the carbon atom to which they are bound denote a bridged bicyclic group of the formula

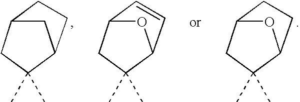

9. A compound of the formula I according to claim 1 wherein the group A denotes a group of the formula

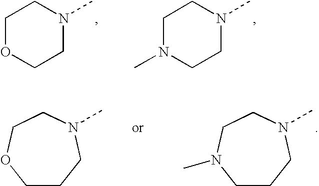

10. A compound of the formula I according to claim 1 wherein the group A denotes a group of the formula

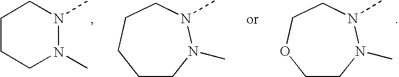

11. A compound of the formula I according to claim 1 wherein R⁶ denotes a bromine atom.

12. A compound of the formula I according to claim 1 wherein R⁶ denotes a chlorine atom.

13. A compound of the formula I according to claim 1 wherein R¹ denotes a fluorine, chlorine or bromine atom or a methyl or trifluoromethyl group.

14. A compound of the formula I according to claim 1 wherein R¹ denotes a hydrogen atom.

15. A physiologically acceptable salt of a compound according to claims 1 to 14.

16. A pharmaceutical composition containing a compound according to claim 1 or a physiologically acceptable salt thereof together with one or more inert carriers and/or diluents.

17. A method for preventing or treating thrombus formation which comprises administering to a patient having a thrombus or prone to thrombus formation an antithrombotic amount of a compound of the formula I according to claims 1-14 or a physiologically acceptable salt thereof.
